(19) United States
(12) Patent Application Publication
(10) Pub. No.: US 2026/0284209 A1
(43) Pub. Date: Sep. 24, 2026

(54) POLYPEPTIDE ADJUVANTS AND USES THEREOF

(71) Applicant: BOARD OF REGENTS, THE UNIVERSITY OF TEXAS SYSTEM, Austin, TX (US)

(72) Inventors: Yon Son Betty KIM, Houston, TX (US); DaeYong LEE, Houston, TX (US); Wen JIANG, Houston, TX (US); Kristin HUNTOON, Houston, TX (US)

(73) Assignee: BOARD OF REGENTS, THE UNIVERSITY OF TEXAS SYSTEM, Austin, TX (US)

(21) Appl. No.: 19/472,622

(22) PCT Filed: Apr. 4, 2024
(86) PCT No.: PCT/US2024/023161

§ 371 Date: Oct. 3, 2025

Related U.S. Application Data
(60) Provisional application No. 63/457,308, filed on Apr. 5, 2023.

(51) Int. Cl. A61K 47/64; A61K 39/39; A61K 47/54; A61K 47/58; A61K 47/61; A61K 49/00; A61K 51/08; A61P 37/04

(52) U.S. Cl. CPC A61K 47/646; A61K 39/39; A61K 47/545; A61K 47/549; A61K 47/58; A61K 47/61; A61K 47/6455; A61K 49/0002; A61K 51/088; A61P 37/04

(57) ABSTRACT

The present disclosure provides polypeptide-based adjuvants that are capable of activating the innate immune system and are capable of generating targeted and precise immunogenicity. Also provided are compositions, pharmaceutical compositions, and vaccines comprising or using the polypeptide-based adjuvants, as well as methods of treating cancer or including or enhancing an immune response using such polypeptide-based adjuvants.

### CROSS REFERENCE TO RELATED APPLICATIONS

This application claims priority to U.S. Provisional Application No. 63/457,308, filed Apr. 5, 2023, the disclosure of which is incorporated herein in its entirety by reference.

### BACKGROUND

The innate immune system plays a crucial role in the first line of host defense against pathogens and malignant transformations. Antigen-presenting cells (APCs) such as dendritic cells (DCs), macrophages and monocytes act as an important bridge between the innate and adaptive immune responses. APCs can recognize and phagocytize malignant cells, thereby promoting tumor-associated antigen presentation to induce cytotoxic effects by T cells. Unfortunately, conditions within the tumor microenvironment often deactivate APCs' immune surveillance by upregulating tumor-promoting factors such as immune checkpoints and anti-inflammatory cytokines and chemokines, and altering the proportion of tumor-support cells.

Stimulating innate immune sensors, such as with cyclic GMP-AMP synthase (cGAS), stimulator of interferon genes (STING), and/or toll-like receptors (TLRs), has been proposed as a strategy to alleviate innate immune inhibition secondary to the suppressive tumor microenvironmental factors, as well as to prime antigen-specific cytotoxic T cells. Previous studies have shown that treatment with conventional immunostimulatory molecules can boost antitumor immune responses. However, conventional adjuvants are capable of activating only a single specific immune sensor, which are often spatially compartmentalized. Moreover, such conventional adjuvants have critical limitations including vulnerability to enzymatic degradation, low water solubility, or safety issues that lead to unsatisfactory therapeutic outcomes.

### BRIEF SUMMARY

In one aspect, the present invention provides a cationic polypeptide-based adjuvant comprising a poly-lysine peptide conjugated to at least one amine containing building block. In one aspect, the present invention provides a polypeptide-based adjuvant comprising the structure of Formula 1:

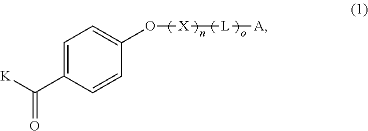

wherein:
K is a poly-lysine peptide;
A is an amine containing building block, an immunogenic agent, an imaging agent, a therapeutic agent, a stabilizing agent, targeting agent, CH₃ or is absent;
X is CH₂ or —CH₂CH₂O;
L is a linker;
wherein n is an integer from 1 to 10, and
is an integer from 0 to 1.

In some aspects, L is selected from the group consisting of substituted or unsubstituted C₁-C₁₀ alkyl, —CH₂CH₂O, streptavidin-biotin, carboxylic acid-amine, carbonates, disulfide bonds, azide-alkyne, azide-DBCO, thiolenes, maleimides, enzyme cleavable peptides, and combinations thereof.

In some aspects, the amine containing building block is attached to the adjuvant by the amine and comprises a compound selected from the group consisting of substituted or unsubstituted heterocyclic compounds, substituted or unsubstituted heteroaryl compounds, linear or branched alkyl amines, substituted or unsubstituted aza-crown ethers, and combinations thereof. In some aspects, the amine containing building block comprises a compound selected from the group consisting of piperazine, 1-methylpiperazine, 1-(2-hydroxyethyl)piperazine, 1-[2-(2-hydroxyethoxy)ethyl]piperazine, morpholine, piperidine, 1-aza-12-crown-4, 1-aza-15-crown-5,1-aza-18-crown-6, dimethyl amine, diethyl amine, dipropyl amine, dibutyl amine, trimethyl amine, triethyl amine, tripropyl amine, tributyl amine, 1-benziimidazole, and combinations thereof. In some aspects, the amine containing building block comprises one or more C₁-C₆ alkyl, halogen, cyano, or hydroxy groups.

In some aspects, the poly-lysine peptide comprises a monomer comprising a substituted oxybenzoyl-L-lysine. In some aspects, the poly-lysine peptide comprises a compound selected from the group consisting of N⁶-4-(2-chloroethyl)oxybenzoyl-L-lysine, N⁶-4-(3-chloropropyl)oxybenzoyl-L-lysine, N⁶-4-(4-chlorobutyl)oxybenzoyl-L-lysine, N⁶-4-(5-chloropentyl)oxybenzoyl-L-lysine, N⁶-4-(6-chlorohexyl)oxybenzoyl-L-lysine, N⁶-4-(7-chloroheptyl)oxybenzoyl-L-lysine, and N⁶-4-(8-chlorooctyl)oxybenzoyl-L-lysine.

In some aspects, the poly-lysine peptide has a molecular weight (g/mol) ranging from about 1,000 to about 1,000,000.

In some aspects, the poly-lysine peptide comprises one or more compounds selected from the group consisting of immunogenic agents, therapeutic agents, imaging agents, stabilizing agents, targeting agents, linkers, and combinations thereof.

In some aspects, the poly-lysine peptide is conjugated to a therapeutic agent by a cleavable linker, or encapsulates a therapeutic agent. In some aspects, the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, mRNA, siRNA, shRNA, plasmid DNA, CpG oligonucleotides, and combinations thereof.

In some aspects, the polypeptide-based adjuvant is conjugated to an imaging agent by a cleavable linker or encapsulates an imaging agent. In some aspects, the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof.

In some aspects, the polypeptide-based adjuvant is conjugated to a stabilizer. In some aspects, the stabilizer is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof.

In some aspects, the polypeptide-based adjuvant is conjugated to a targeting molecule. In some aspects, the targeting molecule is selected from the group consisting of antibodies, nanobodies, aptamers, small molecule-based targeting ligands, and combinations thereof.

In some aspects, the targeting agent is attached to the polypeptide-based adjuvant by a linker molecule. In some aspects, the linker molecule is selected from the group consisting of streptavidin-biotin, carboxylic acid-amine, carbonates, disulfide bonds, azide-alkyne, azide-DBCO, thiolenes, maleimides, enzyme cleavable peptides, and combinations thereof.

In some aspects, the poly-lysine peptide comprises:
a. N⁶-4-(2-hydroxyethyl)oxybenzoyl-L-lysine;
b. N⁶-4-(3-hydroxypropyl)oxybenzoyl-L-lysine;
c. N⁶-4-(4-hydroxybutyl)oxybenzoyl-L-lysine;
d. N⁶-4-(5-hydroxypentyl)oxybenzoyl-L-lysine;
e. N⁶-4-(6-hydroxyhexyl)oxybenzoyl-L-lysine;
f. N⁶-4-(7-hydroxyheptyl)oxybenzoyl-L-lysine; or
g. N⁶-4-(8-hydroxyoctyl)oxybenzoyl-L-lysine.

In some aspects, the polypeptide-based adjuvant is capable of inducing an anti-tumor immune response.

In some aspects, the polypeptide is helical.

In some aspects, the polypeptide-based adjuvant is:

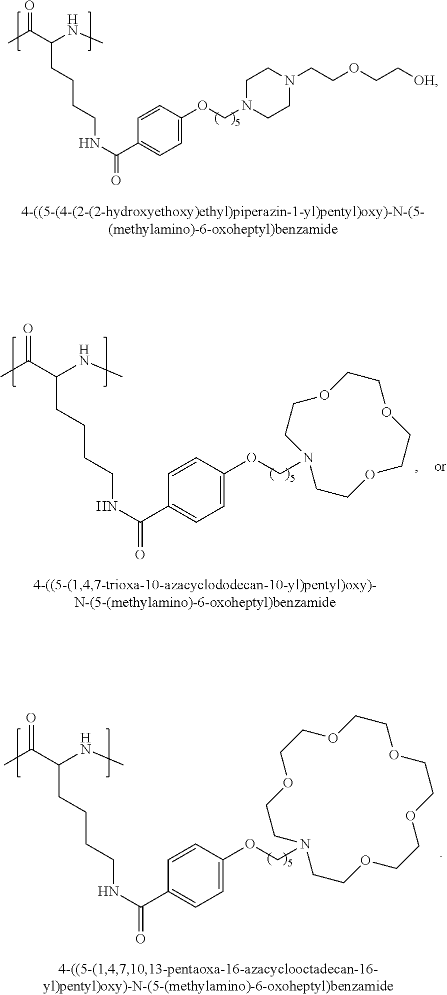

In some aspects described herein is a composition comprising the polypeptide-based adjuvant. In some aspects, the composition further comprises an excipient or carrier. In some aspects, the excipient or carrier comprises an anionic polymer. In some aspects, the carrier is succinylated dextran. In some aspects, the composition is a nanoparticle.

In some aspects described herein is a pharmaceutical composition comprising the polypeptide-based adjuvant and a pharmaceutically acceptable excipient or a pharmaceutically acceptable carrier. In some aspects, the pharmaceutically acceptable excipient or carrier comprises an anionic polymer. In some aspects, the carrier is succinylated dextran.

In some aspects, the composition or pharmaceutical composition comprises one or more compounds selected from the group consisting of immunogenic agents, therapeutic agents, imaging agents, stabilizing agents, targeting agents, linkers, and combinations thereof.

In some aspects, the composition or pharmaceutical composition comprises an immunogenic agent selected from the group consisting of substituted or unsubstituted piperazine, morpholine, piperadine, aza-crown ethers, linear or branched alkyl amines, substituted or unsubstituted benzimidazole, and combinations thereof.

In some aspects, the composition or pharmaceutical composition comprises a therapeutic agent selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, mRNA, siRNA, shRNA, plasmid DNA, CpG oligonucleotides, and combinations thereof.

In some aspects, the composition or pharmaceutical composition comprises an imaging agent selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof.

In some aspects, the composition or pharmaceutical composition comprises a stabilizing agent selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof.

In some aspects, the composition or pharmaceutical composition comprises a targeting agent selected from the group consisting of antibodies, nanobodies, aptamers, small molecules, and combinations thereof. In some aspects, the targeting agent is conjugated to the poly-lysine peptide by a linker. In some aspects, the linker is selected from the group consisting of substituted or unsubstituted C₁-C₁₀ alkyl, —CH₂CH₂O, streptavidin-biotin, carboxylic acid-amine, carbonates, disulfide bonds, azide-alkyne, azide-DBCO, thiolenes, maleimides, enzyme cleavable peptides, and combinations thereof.

In some aspects, the polypeptide-based adjuvant is in the form of a vaccine comprising a pharmaceutically acceptable carrier or diluent suitable for use in a vaccine. In some aspects, the vaccine further comprises an immunogenic compound. In some aspects, the immunogenic compound is selected from the group consisting of piperazine, morpholine, piperadine, aza-crown ether, alkyl amine, benzimidazole, and combinations thereof.

In some aspects, the polypeptide-based adjuvant is in the form of a kit comprising: (i) a polypeptide-based adjuvant (ii) a composition comprising a polypeptide-based adjuvant; (iii) a pharmaceutical composition comprising a polypeptide-based adjuvant; or (iv) a vaccine comprising a polypeptide-based adjuvant, as described herein.

In some aspects described herein is a method of treating cancer in a subject in need thereof, comprising administering to the subject an effective amount of the composition comprising the polypeptide-based adjuvant, the pharmaceutical composition comprising the polypeptide-based adjuvant, or a vaccine comprising the polypeptide-based adjuvant, as described herein. In some aspects, the cancer is selected from the group consisting of a sarcoma, melanoma, carcinoma, glioblastoma or other solid tumors including breast, prostate, lung, kidney and pancreatic tumors. In some aspects, the cancer is breast cancer or a breast cancer metastases. In some aspects, the method further comprises administering a therapeutic agent selected from the group consisting of an anti-PD-1 inhibitor, an anti-PD-L1 inhibitor, an anti-CTLA4 inhibitor, an anti-CD47 inhibitor, an indole 2,3-dioxygenase inhibitor, and combinations thereof.

In some aspects described herein is a method for inducing an immune response or enhancing an immune response in a subject in need thereof, comprising administering to the subject an effective amount of the polypeptide-based adjuvant, the composition comprising the polypeptide-based adjuvant, the pharmaceutical composition comprising the polypeptide-based adjuvant, or a vaccine comprising the polypeptide-based adjuvant, as described herein.

In some aspects described herein is a method of immunizing or conferring a protective immunity against a cancer in a subject in need thereof, comprising administering to the subject the polypeptide-based adjuvant, the composition comprising the polypeptide-based adjuvant, the pharmaceutical composition comprising the polypeptide-based adjuvant, or a vaccine comprising the polypeptide-based adjuvant, as described herein.

In some aspects described herein is a method of delivering a small molecule compound or a polynucleotide to a subject in need thereof, comprising administering the polypeptide-based adjuvant, the composition comprising the polypeptide-based adjuvant, or the pharmaceutical composition comprising the polypeptide-based adjuvant, as described herein. In some aspects, the polypeptide-based adjuvant is conjugated to or encapsulates the small molecule compound or a polynucleotide.

In some aspects described herein, the polypeptide-based adjuvant, composition comprising the polypeptide-based adjuvant, or pharmaceutical composition comprising the polypeptide-based adjuvant is administered orally, intravenously, intraperitoneally, intratumorally, intramuscularly, subcutaneously, or intrathecally.

In some aspects described herein is a method of treating a disease or disorder in a subject in need thereof, comprising administering to the subject an effective amount of a vaccine comprising the polypeptide-based adjuvant as described herein.

In some aspects described herein is a method of making the polypeptide-based adjuvant, the composition comprising the polypeptide-based adjuvant, or the pharmaceutical composition comprising the polypeptide-based adjuvant as described herein, comprising:
(a) making a poly-lysine peptide by synthesizing a lysine monomer followed by polymerization to form a poly-lysine peptide,
(b) conjugating an amine containing building block, and optionally
(c) adding the polypeptide-based adjuvant to an anionic polymer in varying weight ratios to form a nanoparticle.

### BRIEF DESCRIPTION OF THE DRAWINGS

FIG. 1A shows the synthetic scheme for synthesizing polypeptides P1, P2, and P3.

FIG. 1B shows the NMR spectra of starting material poly(4-(5-chloropentyloxy)benzoyl-L-lysine)) in DMSO-d6, P1 in D₂O, P2 in D₂O, and P3 in D₂O.

FIG. 1C shows the circular dichroism spectra of P1, P2, and P3.

FIG. 1D shows the gel permeation chromatography of the starting material, poly(4-(5-chloropentyloxy)benzoyl-L-lysine)).

FIG. 2A shows the synthetic scheme for synthesizing succinylated dextran.

FIG. 2B shows the NMR spectrum of succinylated dextran (DMSO-d6).

FIG. 2C shows the general scheme of forming a P1-loaded nanoparticle (SDP1) based on electrostatic attraction.

FIG. 2D shows a bar graph of the mean diameters and zeta potentials of SDP1 measured by varying different weight ratios.

FIG. 2E shows a bar graph displaying the polydispersity index of the SDP1 nanoparticles based on different weight ratios.

FIG. 3A shows a spectra of the polarization of macrophage phenotype from M2 to M1 upon treatment with P1, P2, and P3 in comparison to valinomycin (V) and moensin (M).

FIG. 3B shows a bar graph quantifying the upregulated expression of the M1 surface markers CD80 and CD86 upon treatment of P1, P2, P3, regardless of macrophage phenotype.

FIG. 3C shows a bar graph quantifying the overproduction of reactive oxygen species (ROS) in the cytosol upon treatment of P1, P2, and P3.

FIG. 3D shows a heat map of M1 and M2 markers in M0, M1, and M2 macrophages evaluated by RT-qPCR, upon treatment of P1, P2, and P3

FIG. 3E shows a bar graph quantifying the relative expression of M1 markers CD80, CD86, iNOS, TNF-α, and IL-1β in M2 macrophages upon treatment of P1, P2, and P3.

FIG. 3F shows a bar graph quantifying the relative expression of M1 markers Ym-1, CD206, Arg-1, and IL-10 in M2 macrophages upon treatment of P1, P2, and P3.

FIG. 3G shows line graphs of the relative cell viability of M0, M1, and M2 macrophages treated with P1, P2, and P3 as evaluated by an MTT assay.

FIG. 3H shows a western blot of expressed proteins related to canonical NF-κB pathways for M1 macrophage polarization upon treatment with P1, P2, or P3 or Control, valinomycin (V), monensin (M), or lipopolysaccharide (LPS).

FIG. 4 shows a bar graph quantifying the relative expression of M1-associated markers (cd80, cd86, nos2, tnfa, and il1b) and M2-associated markers (cd206 and il10) in THP1-derived M2 human macrophage-like cells.

FIG. 5 shows an immunofluorescence staining of p-STING and MyD88 in M2 BMDMs.

FIG. 6A shows a bar graph quantifying the relative expression of macrophage phenotypic markers as determined by RT-qPCR, upon treatment with P1, SD, SDP1 and LPS.

FIG. 6B shows a western blot of cGAS-STING, MyD88, and canonical NF-κB pathways expression upon treatment of P1, SD, SDP1, LPS, and cGAMP, comparatively.

FIG. 7A shows confocal microscopy of the cellular trafficking of P1 and SDP1 out of lysosomes in M2 BMDMs at 30 min, 1 hour, and 3 hours.

FIG. 7B shows confocal microscopy of P1 and SDP1 localized in the ER of M2 BMDMs.

FIG. 7C shows a bar graph showing the calcein uptake upon treatment of P1, SD, SDP1, LPS, and cGAMP.

FIG. 7D shows a bar graph displaying lysosomal pH upon treatment of P1, SD, SDP1, LPS, and cGAMP.

FIG. 8A shows a western blot of ER stress-related proteins upon treatment of P1, SD, SDP1, LPS, and Tunicamycin.

FIG. 8B shows a bar graph of the gene expression ratio of spliced XBP1 (XBP1s) to unspliced XBP1 (XBP1u) upon treatment of P1, SD, SDP1, LPS, and Tunicamycin, as determined by RT-qPCR.

FIG. 9A shows a flow cytometry spectra and bar graph of mitochondrial ROS levels in THP1-derived human macrophage-like cells upon treatment with P1, SD, SDP1, LPS and cGAMP.

FIG. 9B shows a bar graph displaying the cytosolic mtDNA release in THP1-derived M2 human macrophage-like cells.

FIG. 10A shows the release of transcription factor A (TFAM) and double stranded DNA from mitochondria, visualized by confocal laser scanning microscopy.

FIG. 10B shows the measured mitochondrial reactive oxygen species (ROS) upon treatment with P1, SD, SDP1, LPS and cGAMP, as evaluated by flow cytometry.

FIG. 10C shows the mtDNA release upon treatment with P1, SD, SDP1, LPS and cGAMP, quantified by RT-qPCR.

FIG. 10D shows the western blotting of proteins related to cGAS-STING, MyD88 and canonical NF-kB pathways under sodium 4-phenylbutyric acid (4-PBA)-treated conditions, with lipopolysaccharide (LPS) as a TLR agonist, cGAMP:2′,3′-cyclic guanosine monophosphate-adenosine monophosphate as a STING agonist.

FIG. 10E shows the western blotting of proteins related to cGAS-STING, MyD88 and canonical NF-kB pathways under cyclosporin A (CsA)-treated conditions, with lipopolysaccharide (LPS) as a TLR agonist, cGAMP:2′,3′-cyclic guanosine monophosphate-adenosine monophosphate as a STING agonist.

FIG. 11 shows the western blotting of proteins related to cGAS-STING, MyD88 and canonical NF-kB pathways under ER stress inducing conditions with Tunicamycin (Tu) as a stress inducer.

FIG. 12 shows the western blotting of proteins related to the MyD88 pathway in the presence of chloroquine (CQ) and in response to treatment with P1 and SDP1.

FIG. 13A shows the western blotting of proteins related to canonical NF-kB pathways in response to treatment with P1 and SDP1 in STING^{-/-} macrophages.

FIG. 13B shows the western blotting of proteins related to canonical NF-kB pathways in response to treatment with P1 and SDP1 in MyD88^{-/-} macrophages.

FIG. 14A shows the western blotting of proteins related to MyD88 and cGAS-STING pathways in E0771 breast cancer cells in response to treatment with P1, LPS, and cGAMP.

FIG. 14B shows the western blotting of proteins related to MyD88 and cGAS-STING pathways in 4T1 breast cancer cells in response to treatment with P1, LPS, and cGAMP.

FIG. 15A shows phagocytosis activity in SK-BR3, TUBO, and 4T1 breast cancer cell lines treated with P1, SD, SDP1, and LPS, as evaluated by flow cytometry.

FIG. 15B shows bar graphs quantifying phagocytosis (%) in SK-BR3, TUBO, and 4T1 breast cancer cell lines treated with P1, SD, SDP1, and LPS.

FIG. 16A shows phagocytosis activity upon treatment with P1, SD, SDP1, or LPS, as evaluated by flow cytometry.

FIG. 16B shows bar graphs quantifying phagocytosis (%) with P1, SD, SDP1, and LPS, as evaluated by flow cytometry.

FIG. 16C shows phagocytosis of E0771 cells upon treatment with P1, SD, SDP1, and LPS and cross presentation of SIINFEKL-H2Kb, visualized by confocal laser scanning microscopy.

FIG. 16D shows cross-presentation of SIINFEKL-H2Kb peptides on the surface of macrophages, as assessed by flow cytometry to quantify mean fluorescence intensity of SIINFEKL-H2Kb peptides.

FIG. 16E shows spectra and bar graphs of the phagocytosis activity in E0771 cancer cells receiving either P1 or SDP1 upon treatment with sodium 4-phenylbutyric acid (4-PBA) (top) or cyclosporin A (CsA) (bottom).

FIG. 16F shows P1 and SDP1 production of TNF-α and IL-1β in E0771 cancer cells receiving either P1 or SDP1 upon treatment with sodium 4-phenylbutyric acid (4-PBA) or cyclosporin A (CsA).

FIG. 16G shows bar graphs of pro-inflammatory cytokines (TNF-α and IL-1β) and type I interferons (IFN-α and IFN-β) in co-culture conditions upon treatment with P1, SD, SDP1, and LPS.

FIG. 16H shows a bar graph of proliferation (%) of CD4 and CD8 T cells isolated from transgenic OT-II and OT-I mice and co-cultured with P1 and SDP1-treated macrophages and cancer cells.

FIG. 16I shows a bar graph of activated T central memory (T_{CM}) and T effector memory (T_{EM}) subtypes of both CD4 and CD8 T cells from OT-I mice.

FIG. 16J shows a bar graph of activated T central memory (T_{CM}) and T effector memory (T_{EM}) subtypes of both CD4 and CD8 T cells from OT-II mice.

FIG. 17 shows line graphs depicting the relative cell viability of E0771, 4T1, TUBO, and SKBR3 breast cancer cells treated with P1, SD, and SDP1 as evaluated by MTT assay.

FIG. 18A shows line graphs of the body weights of P1-, SDP1-, and cGAMP-treated C57BL/6J mice, which were intravenously given P1, SDP, or cGAMP three times every other day.

FIG. 18B shows a graph depicting the proportion of CD45+ cells in blood plasma after treatment with P1, SDP1, or cGAMP, evaluated by flow cytometry.

FIG. 18C shows bar graphs of levels of blood urea nitrogen (BUN), creatinine, alanine aminotransferase (ALT), and aspartate aminotransferase (AST) in blood plasma of P1-, SGP1-, and cGAMP-treated mice.

FIG. 18D shows a well plate and bar graph displaying the hemolytic activity of various concentration of P1 and SDP1 in red blood cells.

FIG. 19A shows line graphs depicting the tumor volume of E0771 tumor-bearing C57BL/6J mice intravenously injected with HEPES, P1, SDP1, or cGAMP on Days 10, 12, and 14.

FIG. 19B shows Kaplan-Meier survival curves of E0771 tumor-bearing mice treated with HEPES, P1, SDP1, or cGAMP.

FIG. 20A shows a timeline of intravenous sample administration (10 mg/Kg for P1, SDP1 and cGAMP).

FIG. 20B shows a graph depicting growth curves for E0771 tumors in mice after the indicated treatments (n=8, biologically independent mice for each group).

FIG. 20C shows excised tumor tissues from each group 3 days after the last treatment.

FIG. 20D shows Kaplan-Meier survival curves of E0771 tumor-bearing mice show that P1 and SDP1 improved survival. Log-rank (Mantel-Cox) test in comparison to HEPES.

FIGS. 20E-20G show bar graphs determined by flow cytometry of tumor-infiltrating T cells (FIG. 20E), IFN-γ+CD8+ T cells (FIG. 20F) and Treg cells (FIG. 20G) upon treatment with P1, SD, SDP1, and cGAMP.

FIG. 20H shows immunohistologic staining of tumor tissues stained for CD4+, CD8+, and Iba1+ upon treatment with P1, SD, SDP1, and cGAMP.

FIGS. 20I-20K show a bar graphs determined by flow cytometry of tumor-infiltrating myeloid cells upon treatment with P1, SD, SDP1, and cGAMP.

FIG. 21A shows a bar graph of CD8⁺ splenic T cells isolated from spleens of E0771 tumor-bearing mice, evaluated by flow cytometry.

FIG. 21B shows a bar graph of CD4⁻ splenic T cells isolated from spleens of E0771 tumor-bearing mice, evaluated by flow cytometry.

FIGS. 21C and 21D show bar graphs of IFN-γ (FIG. 21C) and IL-2 (FIG. 21D) levels in blood plasma, quantified by enzyme-linked immunosorbent assay.

FIGS. 22A and 22B show bar graphs of tumor-infiltrating immune cells and CD45⁻ cells from E0771 tumors at 1 day after the last treatment, evaluated by flow cytometry.

FIG. 23A shows a diagram of the timeline for tumor establishment and administration of P1 and αPD1.

FIG. 23B shows a graph of growth curves for E0771 tumors from mice after the indicated treatments in FIG. 23A.

FIG. 23C shows excised tumor tissues on day 17 from each treatment group.

FIG. 23D shows Kaplan-Meier survival curves in E0771 tumor-bearing mice from each treatment group.

FIG. 24A shows a timeline for tumor establishment and administration of P1 and αPD1.

FIG. 24B shows a graph of growth curves for 4T1 tumors in mice after the indicated treatments in FIG. 24A.

FIG. 24C shows excised 4T1 tumor tissues on day 17 from each treatment group.

FIG. 24D shows Kaplan-Meier survival curves of 4T1 tumor-bearing mice from each treatment group.

FIGS. 25A-25C show bar graphs in which P1+αPD1 combination increased the population of tumor-infiltrating T cells (FIG. 25A) and IFN-γ+CD8+ T cells (FIG. 25B) along with reducing the population of regulatory T cells (Tregs; FIG. 25C), as evaluated by flow cytometry.

FIG. 25D shows immunofluorescence staining of tumor-infiltrating T cells and Iba1+ cells showing that the combination treatment modulated the tumor microenvironments.

FIGS. 26A-26C shows bar graphs of tumor-infiltrating T lymphocytes (FIG. 26A), IFN-γ+CD8+ T cells (FIG. 26B), and regulatory T cells (Tregs; FIG. 26C).

FIG. 26D shows immunofluorescence staining for CD4, CD8, and Iba1+ in macrophages.

FIGS. 27A-27C show bar graphs in which P1+αPD1 combination treatment promoted M1 macrophage polarization (FIG. 27A) and maturation of dendritic cells (DCs; FIG. 27B) while decreasing the population of myeloid-derived suppressor cells (MDSCs; FIG. 27C), as evaluated by flow cytometry.

FIGS. 28A-28C show bar graphs of profiles of tumor-infiltrating macrophages (FIG. 28A), dendritic cells (FIG. 28B), and myeloid-derived suppressor cells (MDSCs; FIG. 28C), as evaluated by flow cytometry.

FIGS. 29A and 29B shows bar graphs of CD8⁺ T cells (FIG. 29A) and CD4⁺ T cells (FIG. 29B) isolated from the spleens of mice bearing E0771 tumors.

FIGS. 29C and 29D show bar graphs of CD8⁺ T cells (FIG. 29C) and CD4⁺ T cells (FIG. 29D) isolated from the spleens of mice bearing 4T1 tumors.

FIGS. 29E and 29F shows graphs of IFN-γ (FIG. 29E) and IL-2 (FIG. 29F) levels in blood plasma of mice bearing E0771 tumors.

FIGS. 29G and 29H shows graphs of IFN-γ (FIG. 29G) and IL-2 (FIG. 29H) levels in blood plasma of mice bearing 4T1 tumors.

FIG. 30A shows a graph of tumor volume over time in E0771 tumor-bearing mice when STING is not present.

FIG. 30B shows Kaplan-Meier survival curves in E0771 tumor-bearing mice when STING is not present.

FIG. 30C shows harvested tissues of WT and STING^{-/-} tumors on day 16 after treatment with HEPES or P1+αPD1.

FIG. 30D shows a graph of tumor volume over time in E0771 tumor-bearing mice when MyD88 is not present.

FIG. 30E shows Kaplan-Meier survival curves in E0771 tumor-bearing mice when MyD88 is not present.

FIG. 30F shows harvested tissues of WT and MyD88^{-/-} tumors on day 16 after treatment with HEPES or P1+αPD1.

FIGS. 30G and 30H show bar graphs of the relative expression of ifna and ifnb in tumor-homing macrophages (F4/80+) (FIG. 30G) and tumor-homing DCs (CD11c+) (FIG. 30H) with the P1+αPD1 combination treatment in STING−/− mice.

FIGS. 30I and 30J show bar graphs of the relative expression of ifa and ifnb in tumor-homing macrophages (F4/80+) (FIG. 30I) and tumor-homing DCs (CD11c+) (FIG. 30J) with the P1+αPD1 combination treatment in MyD88−/− mice.

FIG. 30K shows immunofluorescence images of phosphorylation of IRF3 (p-IRF3) in tumor-homing macrophages upon P1+αPD1 combination treatment. White arrows identify nuclear translocation.

FIG. 30L shows a bar graph quantification of nucleus translocating p-IRF3⁺ in tumor-homing macrophages upon P1+αPD1 combination treatment.

FIGS. 30M and 30N show bar graph quantification of tumor-infiltrating CD4+ T cell (FIG. 30M) and CD8⁺ T cells (FIG. 30N) with P1+αPD1 combination treatment in wild type, STING−/−, and MyD88−/− mice.

FIG. 30O shows immunofluorescence staining of CD8⁺ and CD4⁺ T cells in E0771 tumors from wild-type (WT), STING−/−, and MyD88−/− mice.

FIGS. 31A and 31B show CD8⁺ T cell populations in splenocytes, as evaluated by flow cytometry (FIG. 31A), on day 17 after tumor inoculation and quantification of CD8 depletion in a bar graph (FIG. 31B).

FIG. 31C shows a graph depicting the growth curve of tumor volume over time after treatment with P1+αPD1.

FIG. 31D shows a graph depicting Kaplan-Meier survival curves of E0771 tumor-bearing mice after treatment with P1+αPD1.

FIG. 31E shows excised tumors from each experimental group of E0771 tumor-bearing mice after treatment with P1+αPD1.

FIG. 31F shows immunofluorescence staining of tumor-infiltrating CD8⁺ T cells of E0771 tumor-bearing mice after treatment with P1+αPD1.

FIGS. 31G-31I shows bar graphs depicting tumor-infiltrating CD8⁺ T cells (FIG. 31G), IFN-γ⁺CD8⁺ T cells (FIG. 31H), and regulatory T cells (Tregs; FIG. 31I) after treatment with P1+αPD1.

FIG. 32 shows a timeline illustrating resection of 4T1-BR4 breast tumors followed by the administration of P1+αPD1 to inhibit spontaneous metastasis.

FIG. 33A shows Kaplan-Meier survival curves of mice with 4T1-BR4 tumor metastases from P1, αPD1, P1+αPD1, and cGAMP+αPD1-treated mice.

FIG. 33B shows representative in vivo bioluminescence images for monitoring metastasis of 4T1-BR4-Luc breast tumor cells after tumor resection.

FIGS. 34A-34C show the synthetic schemes of polypeptide designs by varying hydrophobicity (34A), electrostatic charge (34B), and side chain length (34C).

FIG. 35A shows the chemical structure of cationic polypeptides with different amine-containing analogues.

FIG. 35B shows the western blotting of proteins related to ER stress in bone marrow-derived macrophages in response to treatment with the cationic polypeptides with different amine-containing analogues.

FIG. 35C-F show bar graphs depicting the mtDNA release in bone marrow-derived macrophages (35C), phagocytosis of EO771 breast cancer cells (35D), cross-presentation of model antigen SIINFEKL-H2Kb (35E), and the relative gene expression of pro-inflammatory cytokines (35F) in response to treatment with the cationic polypeptides with different amine-containing analogues.

FIG. 36A shows the chemical structure of polypeptides with different electrolytes.

FIG. 36B shows the western blotting of proteins related to ER stress in release in bone marrow-derived macrophages in response to treatment with the polypeptides with different electrolytes.

FIG. 36C-F show bar graphs depicting the mtDNA release in bone marrow-derived macrophages (36C), phagocytosis of EO771 breast cancer cells (36D), cross-presentation of model antigen SIINFEKL-H2Kb (36E), and the relative gene expression of pro-inflammatory cytokines (36F) in response to treatment with the polypeptides with different electrolytes.

FIG. 37A shows the chemical structure of polypeptides with different side chain lengths.

FIG. 37B shows the western blotting of proteins related to ER stress in release in bone marrow-derived macrophages in response to treatment with the polypeptides with different side chain lengths.

FIG. 37C-F show a bar graphs depicting the mtDNA release in bone marrow-derived macrophages (37C), phagocytosis of EO771 breast cancer cells (37D), cross-presentation of model antigen SIINFEKL-H2Kb (37E), and the relative gene expression of pro-inflammatory cytokines (37F) in response to treatment with the polypeptides with different side chain lengths.

FIG. 38A shows a schemative illustration and corresponding chemical structures of P1, P2, and P3 polypeptides.

FIG. 38B shows fluorescence images of EO771 tumour-bearing mice taken at predetermined times after intravenous injection of IR800CW-tagged P1, P2, or P3 and ex vivo fluorescence images of tumour and major organs harvested at 24 h after administration.

FIG. 38C shows fluorescence images of EO771 tumour tissues excised at 24 h after treatment with IR800CW-tagged P1, P2 or P3.

FIG. 38D a bar graph depicting the total radiation efficiency (near infrared fluorescence signal) of tumour and major organs at 24 h after treatment.

FIG. 38E shows a line graph and table depicting polypeptide concentrations in blood plasma after intravenous administration of IR800CW-tagged P1, P2, or P3 over time and their respective half life and AUC.

FIG. 38F-G show bar graphs depicting the measurement of IR800CW+ fluorescence signals in CD45+ cells (leukocytes) (38F), and immune cell subtypes (38G) by flow cytometry of tumours 24 after treatment with IR800CW-tagged P1, P2, or P3.

FIG. 38H shows immunofluorescence images of IR800CW-tagged P1, P2, or P3 in macrophages and DCs, within tumour microenvironments.

FIG. 38I shows a timeline of treatment with P1, P2, and P3 in EO771 tumor bearing mice.

FIG. 38J shows a line graph depicting the tumor volume over a period of time following treatment with P1, P2, and P3 in EO771 tumor bearing mice.

FIG. 38K shows Kaplan-Meier survival curves following treatment with P1, P2, and P3 in EO771 tumor bearing mice.

FIG. 38L shows excised tumors at day 17 following treatment with P1, P2, and P3 in EO771 tumor bearing mice.

FIG. 39A shows a timeline of intravenous treatments for P1, cGAMP, and CpG.

FIG. 39B shows line graphs depicting the growth curves for EO771 tumours in mice after the indicated treatments.

FIG. 39C shows a timeline for systemic treatments for HEPES, P1, ADU-S100 (a synthetic STING agonist with thiol esters), MSA-2 (non-nucleotide STING agonist), or cGAMP+CpG.

FIG. 39D shows a line graph depicting the tumor volume over a period of time following treatment with HEPES, P1, ADU-S100, MSA-2, and cGAMP+CpG in EO771 tumor bearing mice.

FIG. 39E shows Kaplan-Meier survival curves following treatment with HEPES, P1, ADU-S100, MSA-2, and cGAMP+CpG in EO771 tumor bearing mice.

FIG. 40A shows histological images of heart, spleen, liver, lung, and kidney tissue following intravenous injection of HEPES and P1 in C57BL/6J mice.

FIG. 40B shows box plots depicting levels of blood urea nitrogen (BUN), creatinine, alanine aminotransferase (ALT), and aspartate aminotransferase (AST) in blood plasma of treated mice.

FIG. 41 shows graphs depicting complete blood counts of WBC, white blood cell; RBC, red blood cell; MCH, mean corpuscular hemoglobin; MCV, mean corpuscular volume; MCHC, mean corpuscular hemoglobin concentration; HGB, hemoglobin; HCT, hematocrit; MPV, mean platelet volume; RDW; red cell distribution width; SEGS, segmental neutrophils; EOS, eosinophils; BASOS, basophils; and LUC, large unstained cells following treatment with P1 and HEPES in C57BL/6J mice.

FIG. 42A shows bar graphs depicting the production of IFN7 in tumour-draining lymph nodes and spleen cells following treatment of HEPES, P1, αPD1, P1+αPD1, and cGAMP+αPD1.

FIG. 42B shows bar graphs depicting the production of mature DCs and macrophages in tumour-draining lymph nodes and spleen cells following treatment of HEPES, P1, αPD1, P1+αPD1, and cGAMP+αPD1.

FIG. 43A shows a schematic drawing of treatment induced cell death and the mechanism of SIINFEKL-H2Kb tetramer+CD8+ T cell expansion.

FIG. 43B-43C show spectra and bar graphs of tumor specific T cell response in solid tumors within the tumor microenvironment (43B) and in spleen cells (43C) following treatment of HEPES, P1, αPD1, P1+αPD1, and cGAMP+αPD1.

FIG. 44A shows a timeline depicting the treatment schedule for tumour re-challenge with 4T1-BR4 breast tumour cells after treatments with P1+αPD1.

FIG. 44B shows Kaplan-Meier survival curves of mice with 4T1-BR4 tumour metastases following treatment of HEPES or P1+αPD1.

FIG. 44C shows a line graph depicting the tumor volume over a period of time after re-challenge following treatment with HEPES or P1+αPD1 in 4T1-BR4 breast tumor bearing mice.

FIG. 44D shows a Kaplan-Meier survival curves of mice with 4T1-BR4 tumour metastases after re-challenge following treatment of HEPES or P1+PD1.

### DETAILED DESCRIPTION

The present disclosure provides polypeptide-based adjuvants that are capable of activating the innate immune system and are capable of generating targeted and precise immunogenicity. As described herein, the design of the polypeptide-based adjuvants allows for various helical polypeptide libraries to be synthesized by conjugating different functional moieties to the side chain of the polypeptides. Without wishing to be bound by any one theory, the helical polypeptides comprising amine-containing building blocks induces an anti-tumor immune response by promoting mtDNA release. Additionally, polypeptide-based adjuvants described herein having a cationic charge is capable of promoting, among other things, intracellular internalization and encapsulation of therapeutic cargoes such as therapeutic agents, including but not limited to, small molecule compounds and polynucleotides. Thus, the polypeptide-based adjuvants described herein provide a versatile platform to treat, for example, several types of cancers by inducing anti-tumor innate immunity, co-delivering therapeutic cargos, and/or engineering immune cells in situ with anti-tumor functions via transfection.

Unlike conventional adjuvants that are capable of activating only a single, specific immune sensor, the polypeptide-based adjuvants described herein are capable of agonizing at least two key innate immune sensors: toll-like receptor (TLR) and cyclic GMP-AMP synthase (cGAS)-stimulator of interferon gene (STING) pathways. Without wishing to be bound by any one theory, the polypeptide-based adjuvants described herein having a cationic helical conformation are preferentially taken up by macrophages or dendritic cells and accumulate in endoplasmic reticulum (ER), and the resulting ER stress permeabilizes the mitochondrial outer membrane. Mitochondrial DNA (mtDNA) release from a permeabilized mitochondrial outer membrane is then capable of stimulating both myeloid differentiation primary response 88 (a TLR mediator), and cGAS-STING cascade, thereby promoting secretion of pro-inflammatory cytokines and type I interferons to activate both innate and adaptive immunity. Thus, the polypeptide-based adjuvants described herein can simultaneously agonize both the important innate immune sensors to boost systemic long-lived anti-tumor immunity without corresponding toxicity.

### Definitions

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

The term “about” as used in connection with a numerical value throughout the specification and the claims denotes an interval of accuracy, familiar and acceptable to a person skilled in the art. In certain aspects, such interval of accuracy is ±10%. In other aspects, such interval of accuracy is ±5%.

As used herein, the terms “subject” and “subjects” are synonymous with and can be used interchangeably with “patient” and “patients.” The term “subject” includes any human or nonhuman animal. The term “nonhuman animal” includes, but is not limited to, vertebrates such as dogs, cats, horses, cows, pigs, boar, sheep, goat, buffalo, bison, llama, deer, elk and other large animals, as well as their young, including calves and lambs, and to mice, rats, rabbits, guinea pigs, primates such as monkeys and other experimental animals. Within animals, mammals are preferred, most preferably, valued and valuable animals such as domestic pets, race horses and animals used to directly produce (e.g., meat) or indirectly produce (e.g., milk) food for human consumption, although experimental animals are also included. In specific aspects, the subject is a human. Thus, the present disclosure is applicable to clinical, veterinary and research uses.

The terms “administer” or “administering,” as used herein, refer to the physical introduction of a composition comprising a polypeptide-based adjuvant described herein to a subject, using any of the various methods and delivery systems known to those skilled in the art. Routes of administration include oral, intravenous, intramuscular, subcutaneous, intraperitoneal, spinal or other parenteral routes of administration, for example by injection or infusion.

The term “pharmaceutically acceptable salt” means those salts of compounds that are safe and effective for use in subjects and that possess the desired biological activity.

Pharmaceutically acceptable salts of a basic compound can be salts of organic or inorganic acids. In some aspects, the organic and inorganic acids include but are not limited to hydrochloric acid, hydrobromic acid, sulfuric acid, phosphoric acid, citric acid, maleic acid, mandelic acid, succinic acid and methanesulfonic acid. (see generally, J. Pharm. Sci., 66, 2 (1977), which is incorporated herein by reference in its entirety).

The terms “therapeutically effective amount” or “effective dose” as used herein refer to an amount that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. When applied to an individual active ingredient, administered alone, the term refers to that ingredient alone. When applied in combination, the term refers to the combined amounts of the active ingredients that result in the therapeutic effect, whether administered in combination, serially, or simultaneously. An effective amount can be administered in one dosage or can be divided into multiple dosages, the total of such dosages being the effective amount. For example, an effective amount can be provided in two separate administrations over a period of time that, in aggregate, provide the effective amount of the formulation. In the case of a cancer or other proliferation disorder, the therapeutically effective amount of the agent may reduce (i.e., retard to some extent and preferably stop) unwanted cellular proliferation; reduce the number of cancer cells; reduce the tumor size; inhibit (i.e., retard to some extent and preferably stop) cancer cell infiltration into peripheral organs; inhibit (i.e., retard to some extent and preferably stop) tumor metastasis; inhibit, to some extent, tumor growth; modulate protein methylation in the target cells; and/or relieve, to some extent, one or more of the symptoms associated with the cancer. To the extent the administered compound or composition prevents growth and/or kills existing cancer cells, it may be cytostatic and/or cytotoxic.

In addition, the terms “effective” and “effectiveness” with regard to a treatment disclosed herein includes both pharmacological effectiveness and physiological safety. Pharmacological effectiveness refers to the ability of the drug to promote, e.g., cancer regression in the patient. Physiological safety refers to the level of toxicity, or other adverse physiological effects at the cellular, organ and/or organism level (adverse effects) resulting from administration of the drug.

The ability of a therapeutic agent to promote disease regression, e.g., cancer regression, can be evaluated using a variety of methods known to the skilled practitioner, such as in human subjects during clinical trials, in animal model systems predictive of efficacy in humans, or by assaying the activity of the agent in in vitro assays.

In general, the terms “treat,” “treating,” or “treatment” refer to countering the effects caused as a result of the disease or pathological condition of interest in a subject including (i) inhibiting the progress of the disease or pathological condition, in other words, slowing or stopping the development or progression thereof, or one or more symptoms of such disorder or condition; (ii) relieving the disease or pathological condition, in other words, causing said disease or pathological condition, or the symptoms thereof, to regress; (iii) stabilizing the disease or pathological condition or one or more symptoms of such disorder or condition, (iv) reversing the disease or pathological condition or one or more symptoms of such disorder or condition to a normal state, (v) preventing the disease or pathological condition or one or more symptoms of such disorder or condition, and (vi) any combination thereof. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term a “cancer,” as used herein, refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. The term “tumor” refers to a solid cancer. The term “carcinoma” refers to a cancer of epithelial origin.

The term “alkyl” as used herein means normal, secondary, or tertiary, linear, branched or straight hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, and n-icosyl. In some aspects, the term alkyl refers to, e.g., C₁₋₁₀ alkyl (C₁₋₁₀ hydrocarbons), C₁₋₉ alkyl (C₁₋₉ hydrocarbons), or C₁₋₆ alkyl (C₁₋₆ hydrocarbons), as further defined herein below.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain.

The term “alkyne” or “alkynyl” as used herein refers to C₂-C₁₈ normal, secondary, tertiary, linear, branched or straight hydrocarbon with at least one site (usually 1 to 3, preferably 1) of unsaturation, namely a carbon-carbon, sp triple bond. Examples include, but are not limited to: ethynyl (—C≡CH), 3-ethyl-cyclohept-1-ynylene, and 1-propynyl (propargyl, —CH₂C≡CH). In some aspects, the term alkynyl refers to C₂₋₁₂ alkynyl (C₂₋₁₂ hydrocarbons), C₂₋₉ alkynyl (C₂₋₉ hydrocarbons), or C₂₋₆ alkynyl (C₂₋₆ hydrocarbons), as further defined herein above with at least one site (usually 1 to 3, preferably 1) of unsaturation, namely a carbon-carbon, sp triple bond.

The term “heterocycle” or “heterocyclic” or “heterocyclyl” as used herein refer to non-aromatic, fully saturated or partially unsaturated ring system of 3 to 18 atoms including at least one N, O, S, or P (for example, 3 to 7 member monocyclic, 7 to 11 member bicyclic, or comprising a total of 3 to 10 ring atoms). Each ring of the heterocycle or heterocyclyl may have 1, 2, 3 or 4 heteroatoms selected from N, O and/or S, where the N and S heteroatoms may optionally be oxidized and the N heteroatoms may optionally be quaternized; and wherein at least one carbon atom of heterocyclyl can be oxidized to form at least one C═O. The heterocycle may be attached at any heteroatom or carbon atom of the ring or ring system, where valence allows. The rings of multi-ring heterocyclyls or heterocycles may be fused, bridged and/or joined through one or more spiro atoms. Fused systems of a heterocycle or heterocyclyl with an aryl ring are considered as heterocycle or heterocyclyl irrespective of the ring that is bound to the core structure. Fused systems of a heterocycle or heterocyclyl with a heteroaryl ring are considered as heteroaryl irrespective of the ring that is bound to the core structure.

The term “heteroaryl” refers to an aromatic ring system of 5 to 18 atoms including at least one N, O, S, or P, containing 1 or 2 rings which can be fused together or linked covalently, each ring typically containing 5 to 6 atoms; at least one of said rings is aromatic, where the N and S heteroatoms may optionally be oxidized and the N heteroatoms may optionally be quaternized, and wherein at least one carbon atom of said heteroaryl can be oxidized to form at least one C═O. Fused systems of a heteroaryl ring with a cycloalkyl ring, or a cycloalkenyl ring, or a cycloalkynyl ring, are considered as heteroaryl irrespective of the ring that is bound to the core structure. Fused systems of a heteroaryl ring with a heterocycle are considered as heteroaryl irrespective of the ring that is bound to the core structure. Fused systems of a hetero aryl ring with an aryl ring are considered as heteroaryl irrespective of the ring that is bound to the core structure.

As used herein with respect to a substituting group, and unless otherwise stated, the terms “substituted” such as in “substituted alkyl”, “substituted alkenyl”, substituted alkynyl”, “substituted aryl”, “substituted heteroaryl”, “substituted heterocyclyl”, “substituted arylalkyl”, “substituted heteroaryl-alkyl”, “substituted heterocyclyl-alkyl” and the like refer to the chemical structures defined herein, and wherein the said alkyl, alkenyl, alkynyl, group and/or the said aryl, heteroaryl, or heterocyclyl may be optionally substituted with one or more substituents (preferable 1, 2, 3, 4, 5 or 6), meaning that one or more hydrogen atoms are each independently replaced with at least one substituent. Typical substituents include, but are not limited to and are being independently selected from, the group consisting of halogen, amino, hydroxyl, sulfhydryl, alkyl, alkoxy, alkenyl, alkenyloxy, alkynyl, alkynyloxy, cycloalkyl, cycloalkenyl, cycloalkynyl, disulfide, heteroalkyl, heteroalkenyl, heteroalkynyl, aryl, heteroaryl, heterocyclyl, arylalkyl, arylalkenyl, arylalkynyl, cycloalkyl-alkyl, cycloalkylalkenyl, cycloalkylalkynyl, heteroaryl-alkyl, heterocyclyl-alkyl, heteroaryl-alkenyl, heterocyclyl-alkenyl and heteroaryl-alkynyl, heterocyclyl-alkynyl, —X, —Z, —O⁻, —OZ, ═O, —SZ, —S⁻, ═S, —NZ₂, —N⁺Z₃, ═NZ, ═N—OZ, —CX₃ (e.g. trifluoromethyl), —CN, —OCN, —SCN, —N═C═O, —N═C═S, —NO, —NO₂, ═N₂, —N₃, —NZC(O)Z, —NZC(S)Z, —NZC(O)O⁻, —NZC(O)OZ, —NZC(S)OZ, —NZC(O)NZZ, NZC(NZ)Z, NZC(NZ)NZZ, —C(O)NZZ, —C(NZ)Z, —S(O)₂O⁻, —S(O)₂OZ, —S(O)₂Z, —OS(O)₂OZ, —OS(O)₂Z, —OS(O)₂O—, —S(O)₂NZZ, —S(O)(NZ)Z, —S(O)Z, —OP(O)(OZ)₂, —P(O)(OZ)₂, —P(O)(O⁻)₂, —P(O)(OZ)(O⁻), —P(O)(OH)₂, —C(O)Z, —C(O)X, —C(S)Z, —C(O)OZ, —C(O)O⁻, —C(S)OZ, —C(O)SZ, —C(S)SZ, —C(O)NZZ, —C(S)NZZ, —C(NZ)NZZ, —OC(O)Z, —OC(S)Z, —OC(O)O⁻, —OC(O)OZ, —OC(S)OZ, wherein each X is independently a halogen selected from F, Cl, Br, or I; and each Z is independently —H, alkyl, alkenyl, alkynyl, heteroalkyl, heteroalkenyl, heteroalkynyl, aryl, heteroaryl, heterocyclyl, cycloalkyl, protecting group or prodrug moiety, while two Z bonded to a nitrogen atom can be taken together with the nitrogen atom to which they are bonded to form a heteroaryl, or heterocyclyl. Alkyl(ene), alkenyl(ene), and alkynyl(ene) groups may also be similarly substituted.

The term “ligand” as used herein refers to a molecule or other chemical entity having a capacity for binding to a target. A ligand can comprise a peptide, an oligomer, a nucleic acid (e.g., an aptamer), a small molecule (e.g., a chemical compound), an antibody or fragment thereof, nucleic acid-protein fusion, and/or any other affinity agent. Thus, a ligand can come from any source, including libraries, particularly combinatorial libraries, such as the aptamer libraries, phage display libraries, or any other library as would be apparent to one of ordinary skill in the art after review of the disclosure of the present invention presented herein.

The term “conjugated” refers to the state of two or more components of a molecule being joined, attached, connected, or otherwise coupled to form a single molecule or the act of making two molecules associated with each other to form a single molecule by creating an association, linkage, attachment, and/or any other connection between the two molecules.

The term “linker,” as used herein, refers to a chemical structure between two molecules or moieties or between a molecule and a moiety, thus linking the two. In some aspects, the linker refers to compounds used to provide structural stability or assisted molecular binding reactions such as protein-protein, protein-peptide, protein-polymer, polymer-small molecule, peptide/protein-small molecule interactions, immobilization for assay or purification, and various peptide-nucleic acid and nucleic acid-nucleic acid binding. Typically, the linker comprises a functional group, such as primary amines, thiols, acids, alcohols, azides, alkynes, and halides. In some aspects, maleimide (thiol-reactive), succinimidyl ester (NHS), or Isothiocyanate (ITC) groups reactive with amines may be used.

The term “cleavable linker” refers to linkers that can be cleaved under mild conditions, i.e. conditions under which the activity of the compound is not affected. Many known linkers fall in this category and are described below. Disulfide containing linkers are linkers cleavable through disulfide exchange, which can occur under physiological conditions. Acid-labile linkers are linkers cleavable at acid pH. For example, certain intracellular compartments, such as endosomes and lysosomes, have an acidic pH (pH 4-5), and provide conditions suitable to cleave acid-labile linkers. Linkers that are photo-labile are useful at the body surface and in many body cavities that are accessible to light. Furthermore, infrared light can penetrate tissue. Some linkers can be cleaved by peptidases. Only certain peptides are readily cleaved inside or outside cells, see e.g. Trouet et al., 79 Proc. Natl. Acad. Sci. USA, 626-629 (1982) and Umemoto et al. 43 Int. J. Cancer, 677-684 (1989). Furthermore, peptides are composed of α-amino acids and peptidic bonds, which chemically are amide bonds between the carboxylate of one amino acid and the α-amino group of a second amino acid. Other amide bonds, such as the bond between a carboxylate and the c-amino group of lysine, are understood not to be peptidic bonds and are considered non-cleavable. Some linkers can be cleaved by esterases. Again only certain esters can be cleaved by esterases present inside or outside cells. Esters are formed by the condensation of a carboxylic acid and an alcohol. Simple esters are esters produced with simple alcohols, such as aliphatic alcohols, and small cyclic and small aromatic alcohols.

The term “poly-lysine peptide” as used herein refers to an organic polymer comprising any number of lysine residues bonded together by peptide bonds.

The term “CD47 inhibitor” as used herein refers to any compound or composition that directly or indirectly inhibits CD47 expression and/or activity. In some aspects described herein, the CD47 inhibitor is an anti-CD47 antibody, including, but not limited to, magrolimab, TG-1801 (NI-1701) (anti-CD47/anti-CD19), or an antigen-binding portion thereof, or an antibody that cross-competes with binding to human CD47, or an antibody that that binds to the same epitope as magrolimab, TG-1801 (NI-1701) (anti-CD47/anti-CD19), or an antigen-binding portion thereof.

The term “PD-1 inhibitor” as used herein refers to any compound or composition that directly or indirectly inhibits PD-1 expression and/or activity. “Programmed Death-1” (PD-1) refers to an immunoinhibitory receptor belonging to the CD28 family. PD-1 is expressed predominantly on previously activated T cells in vivo, and binds to two ligands, PD-L1 and PD-L2. In some aspects described herein, the PD-1 inhibitor is an anti-PD-1 antibody, including, but not limited to, nivolumab, pembrolizumab, cemiplimab, spartalizumab (PDR001), sintilimab, tislelizumab, or geptanolimab (CBT-501), or an antigen-binding portion thereof, or an antibody that cross-competes with binding to human PD-1, or an antibody that that binds to the same epitope as nivolumab, pembrolizumab, cemiplimab, spartalizumab (PDR001), sintilimab, tislelizumab, or geptanolimab (CBT-501), or an antigen-binding portion thereof.

The term “PD-L1 inhibitor” as used herein refers to any compound or composition that directly or indirectly inhibits PD-L1 expression and/or activity. “Programmed Death Ligand-i” (PD-L1) is one of two cell surface glycoprotein ligands for PD-1 (the other being PD-L2) that downregulate T cell activation and cytokine secretion upon binding to PD-1. In some aspects described herein, the PD-L1 inhibitor is an anti-PD-L1 antibody, including, but not limited to avelumab, atezolizumab, durvalumab, or an antigen-binding portion thereof, or an antibody that cross-competes with binding to human PD-L1, or an antibody that that binds to the same epitope as avelumab, atezolizumab, durvalumab, or an antigen-binding portion thereof.

The term “CTLA4 inhibitor” as used herein refers to any compound or composition that directly or indirectly inhibits CTLA4 expression and/or activity. In some aspects described herein, the CTLA4 inhibitor is an anti-CTLA4 antibody, including, but not limited to ipilimumab, or an antigen-binding portion thereof, or an antibody that cross-competes with binding to human CTLA4, or an antibody that that binds to the same epitope as ipilimumab, or an antigen-binding portion thereof.

The term “indole 2,3-dioxygenase inhibitor” as used herein refers to any compound or composition that directly or indirectly inhibits indole 2,3-dioxygenase expression and/or activity. In some aspects described herein, the indole 2,3-dioxygenase inhibitor is a compound, including, but not limited to Indoximod (1-methyl-D-tryptophan, 1MT, NLG-8189), Epacadostat (INCB024360), Linrodostat mesylate (BMS-986205), Navoximod (GDC-0919, NLG-919), PF-0684003, or pharmaceutically acceptable salts thereof.

The term “antibody” (Ab) as used herein includes, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “antibody” includes, by way of example, monoclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

The term “nanobody” refers to a variable region of a heavy chain of an antibody, and is a construct having only one heavy chain variable region. It is the smallest antigen-binding fragment with complete function. Generally, the antibodies with a natural deficiency of the light chain and the heavy chain constant region 1 (CH1) are first obtained, the variable regions of the heavy chain of the antibody are therefore cloned to construct a single domain antibody (VHH) or nanobody consisting of only one heavy chain variable region.

As used herein, the term “variable” refers to the certain portions of the variable region of an antibody or fragment thereof, for example nanobodies for scFv, which forms the binding and specificity of various specific antibodies to their particular antigen. However, variability is not uniformly distributed throughout the variable region. It is concentrated in three segments called complementarity-determining regions (CDRs) or hypervariable regions in the variable regions of the light and heavy chain. The more conserved part of the variable region is called the framework region (FR). The variable regions of the natural heavy and light chains each contain four FR regions, which are substantially in a β-folded configuration, joined by three CDRs which form a linking loop, and in some cases can form a partially β-folded structure.

The term “aptamers” refer to oligonucleotides having specific binding regions that are capable of forming complexes with an intended target molecule in an environment wherein other substances in the same environment are not complexed to the oligonucleotide. Aptamers have been described in, e.g., Sun, H. et al. Oligonucleotide Aptamers: New Tools for Targeted Cancer Therapy. Molecular Therapy—Nucleic Acids. 2014. 3, e182.

The term “small molecule” refers to an organic compound having a molecular weight of less than about 900 Daltons, or less than about 500 Daltons. The term includes agents having the desired pharmacological properties, and includes compounds that can be taken orally or by injection.

### Polypeptide-Based Adjuvants and Compositions

As described herein, the present disclosure provides polypeptide-based adjuvants comprising a poly-lysine peptide conjugated to an amine containing building block. In aspects, the polypeptide-based adjuvants are cationic. In aspects, the polypeptide-based adjuvants are helical. In aspects, the polypeptide-based adjuvants are cationic and helical. In aspects, the polypeptide-based adjuvants are capable of inducing an immune response, e.g., an anti-tumor immune response.

As also described herein, the present disclosure provides polypeptide-based adjuvants comprising the structure of Formula 1:

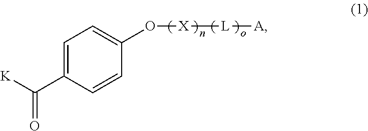

wherein:
K is a poly-lysine peptide;
A is an amine containing building block, an immunogenic agent, an imaging agent, a therapeutic agent, a stabilizing agent, targeting agent, CH₃ or is absent;
X is CH₂ or —CH₂CH₂O;
L is a linker;
wherein n is an integer from 1 to 10, and
o is an integer from 0 to 1. In aspects, the polypeptide-based adjuvants comprising the structure of Formula 1 are cationic. In aspects, the polypeptide-based adjuvants comprising the structure of Formula 1 are helical. In aspects, the polypeptide-based adjuvants comprising the structure of Formula 1 are cationic and helical. In aspects, the polypeptide-based adjuvants comprising the structure of Formula 1 are capable of inducing an immune response, e.g., an anti-tumor immune response.

In some aspects, the polypeptide-based adjuvant comprises the structure of Formula 1 and L is selected from the group consisting of substituted or unsubstituted C₁-C₁₀ alkyl, —CH₂CH₂O, streptavidin-biotin, carboxylic acid-amine, carbonates, disulfide bonds, azide-alkyne, azide-DBCO, thiolenes, maleimides, enzyme cleavable peptides, and combinations thereof.

The polypeptide-based adjuvants described herein comprise an amine containing building block, wherein the amine containing building block comprises a compound selected from the group consisting of substituted or unsubstituted heterocyclic compounds, substituted or unsubstituted heteroaryl compounds, linear or branched alkyl amines, substituted or unsubstituted aza-crown ethers, and combinations thereof. In some aspects, the amine containing building block comprises one or more of a C₁-C₆ alkyl, halogen, cyano, or hydroxy group. In aspects, the amine containing building block comprises a compound selected from the group consisting of piperazine, 1-methylpiperazine, 1-(2-hydroxyethyl)piperazine, 1-[2-(2-hydroxyethoxy)ethyl]piperazine, morpholine, piperidine, 1-aza-12-crown-4, 1-aza-15-crown-5,1-aza-18-crown-6, dimethyl amine, diethyl amine, dipropyl amine, dibutyl amine, trimethyl amine, triethyl amine, tripropyl amine, tributyl amine, 1-benziimidazole, and combinations thereof.

The polypeptide-based adjuvants described herein comprise a substituted oxybenzoyl-L-lysine. In aspects, the substituted oxybenzoyl-L-lysine is selected from the group consisting of 4-(2-chloroethyl)oxybenzoyl-L-lysine, 4-(3-chloropropyl)oxybenzoyl-L-lysine, 4-(4-chlorobutyl)oxybenzoyl-L-lysine, 4-(5-chloropentyl)oxybenzoyl-L-lysine, 4-(6-chlorohexyl)oxybenzoyl-L-lysine, 4-(7-chloroheptyl)oxybenzoyl-L-lysine, and 4-(8-chlorooctyl)oxybenzoyl-L-lysine.

In aspects of the polypeptide-based adjuvants described herein, the poly-lysine peptide has a molecular weight (g/mol) ranging from about 1,000 to about 1,000,000. In some aspects the polypeptide-based adjuvant has a molecular weight (g/mol) in the range from about 1,000 to about 1,000,000, from about 5,000 to about 1,000,000, from about 10,000 to about 1,000,000, from about 15,000 to about 1,000,000, from about 20,000 to about 1,000,000, from about 25,000 to about 1,000,000, from about 30,000 to about 1,000,000, from about 35,000 to about 1,000,000, from about 40,000 to about 1,000,000, from about 45,000 to about 1,000,000, from about 50,000 to about 1,000,000, from about 55,000 to about 1,000,000, from about 60,000 to about 1,000,000, from about 65,000 to about 1,000,000, from about 70,000 to about 1,000,000, from about 75,000 to about 1,000,000, from about 80,000 to about 1,000,000, from about 90,000 to about 1,000,000, from about 100,000 to about 1,000,000, from about 150,000 to about 1,000,000, from about 200,000 to about 1,000,000, from about 300,000 to about 1,000,000, from about 400,000 to about 1,000,000, from about 500,000 to about 1,000,000, from about 600,000 to about 1,000,000, from about 700,000 to about 1,000,000, from about 800,000 to about 1,000,000, from about 10,000 to about 900,000, from about 20,000 to about 800,000, from about 30,000 to about 700,000, from about 40,000 to about 600,000, from about 50,000 to about 500,000, from about 60,000 to about 400,000, from about 70,000 to about 300,000, from about 80,000 to about 100,000.

In some aspects, the polypeptide-based adjuvant has a molecular weight of about 1,000, about 5,000, about 10,000, about 15,000, about 20,000, about 25,000, about 50,000, about 75,000, about 100,000, about 150,000, about 200,000, about 250,000, about 350,000, about 450,000, about 500,000, about 550,000, about 600,000, about 650,000, about 700,000, about 750,000, about 800,000, about 850,000, about 900,000, about 950,000, about 1,000,000, or a range between any two of the preceding values.

In aspects of the polypeptide-based adjuvants described herein, the poly-lysine peptide has a number of lysine residues ranging from about 2 to about 4,000. In some aspects the the poly-lysine peptide has a number of lysine residues in the range from about 2 to about 3,500, from about 2 to about 3,000, from about 2 to about 2,500, from about 2 to about 2,000, from about 2 to about 1,500, from about 2 to about 1,000, from about 2 to about 750, from about 2 to about 700, from about 2 to about 650, from about 2 to about 600, from about 2 to about 550, from about 2 to about 500, from about 2 to about 450, from about 2 to about 400, from about 2 to about 350, from about 2 to about 300, from about 2 to about 250, from about 2 to about 200, from about 2 to about 150, from about 2 to about 100, from about 2 to about 75, from about 2 to about 50, from about 2 to about 40, from about 2 to about 45, from about 2 to about 30, from about 2 to about 25, from about 2 to about 20, from about 2 to about 15, from about 2 to about 10, from about 2 to about 5.

In some aspects, the poly-lysine peptide has a number of lysine residues of about 2, about 5, about 10, about 15, about 20, about 25, about 50, about 75, about 100, about 150, about 200, about 250, about 350, about 450, about 500, about 600, about 700, about 800, about 900, about 1,000, about 1,500, about 2,000, about 2,500, about 3,000, about 3,500, about 4,000 or a range between any two of the preceding values.

In aspects of the polypeptide-based adjuvants described herein, the poly-lysine peptide comprises one or more building blocks selected from the group consisting of immunogenic agents, therapeutic agents, imaging agents, stabilizing agents, targeting agents, linkers, and combinations thereof. In some aspects, the immunogenic agent is selected from the group consisting of piperazine, morpholine, piperadine, aza-crown ether, alkyl amine, benzimidazole, and combinations thereof. In some aspects, the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, and combinations thereof. In some aspects, the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof. In some aspects, the stabilizing agent is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof. In some aspects, the targeting agent is selected from the group consisting of antibodies, nanobodies, aptamers, small molecules, and combinations thereof. In some aspects, the targeting agent is conjugated to the polypeptide by a linker. In some aspects, the linker is selected from the group consisting of streptavidin-biotin, carboxylic acid-amines, disulfides, azide-alkynes, azide-DBCO, thiolenes, and combinations thereof.

In aspects of the polypeptide-based adjuvants described herein, the poly-lysine peptide is conjugated to or encapsulates a therapeutic agent. In some aspects, the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, and combinations thereof.

In aspects of the polypeptide-based adjuvants described herein, the poly-lysine peptide is conjugated to or encapsulates an imaging agent. In some aspects, the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof.

In aspects of the polypeptide-based adjuvants described herein, the polypeptide-based adjuvant is conjugated to a stabilizer. In some aspects, the stabilizer is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof.

In aspects described herein are compositions comprising the polypeptide-based adjuvants described herein, wherein the composition comprises an excipient, a carrier, or a vehicle.

In aspects described herein, the polypeptide-based adjuvants are in the form of nanoparticles. Such nanoparticles can form through self-association and/or through combination with a particle-forming or associating agent, such as an anionic compound. Nanoparticles can also be formed, for example, through physical mixing methods such as vortexing, pipetting, and sonicating. In some aspects, a nanoparticle described herein comprises a polypeptide-based adjuvant and anionic compound, such as a succinylated dextran. As described herein, nanoparticles comprising a polypeptide-based adjuvant herein encapsulate an agent, such as an immunogenic agent, a therapeutic agent, or an imaging agent. In some aspects the nanoparticles comprising a polypeptide-based adjuvant herein further comprise particle stabilizers, including but not limited to, F-127, polyvinyl alcohol, tween-20 or tween-80.

### Pharmaceutical Compositions

As described herein, the present disclosure provides pharmaceutical compositions comprising a polypeptide-based adjuvant comprising a poly-lysine peptide conjugated to an amine containing building block according to the present disclosure or a polypeptide-based adjuvant that comprises the structure of Formula 1 according to the present disclosure. In aspects, the pharmaceutical composition comprises a polypeptide-based adjuvant and a pharmaceutically acceptable excipient or a pharmaceutically acceptable carrier. Such a carrier can be selected from pharmaceutically acceptable excipients and auxiliaries. The terms “pharmaceutically acceptable excipient,” “pharmaceutically acceptable carrier,” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described, for example, in Remington's Pharmaceutical Sciences, Mack Publishing Co., Easton, PA, 23rd ed. 2020.

In some aspects of the pharmaceutical compositions described herein, the pharmaceutically acceptable excipient or carrier comprises an anionic polymer. In some aspects, the pharmaceutically acceptable excipient or carrier comprises a succinylated dextran.

Pharmaceutical compositions within the scope of the present disclosure include all compositions where a polypeptide-based adjuvant is combined with one or more pharmaceutically acceptable excipients or pharmaceutically acceptable carriers. In certain aspects, a polypeptide-based adjuvant is present in the composition in an amount that is effective to achieve its intended therapeutic purpose as described herein. While individual needs may vary, a determination of optimal ranges of effective amounts of polypeptide-based adjuvant is within the skill of the art. Typically, a polypeptide-based adjuvant can be administered to a subject, e.g., a human, at a dose of from 1 to 50 mg/Kg. In some aspects the polypeptide-based adjuvant is administered at a dose in the range from about 1 to about 50 mg/Kg, from about 2 to about 50 mg/Kg, from about 5 to about 50 mg/Kg, from about 10 to about 50 mg/Kg, from about 15 to about 50 mg/Kg, from about 20 to about 50 mg/Kg, from about 25 to about 50 mg/Kg, from about 30 to about 50 mg/Kg, from about 35 to about 50 mg/Kg, from about 40 to about 50 mg/Kg, from about 45 to about 50 mg/Kg, from about 1 to about 45 mg/Kg, from about 2 to about 40 mg/Kg, from about 4 to about 35 mg/Kg, from about 6 to about 30 mg/Kg, from about 8 to about 25 mg/Kg, from about 10 to about 20 mg/Kg, from about 12 to about 15 mg/Kg.

In some aspects the polypeptide-based adjuvant is administered at a dose of about 1 mg/Kg, about 2 mg/Kg, about 3 mg/Kg, about 4 mg/Kg, about 5 mg/Kg, about 6 mg/Kg, about 7 mg/Kg, about 8 mg/Kg, about 9 mg/Kg, about 10 mg/Kg, about 15 mg/Kg, about 20 mg/Kg, about 25 mg/Kg, about 30 mg/Kg, about 35 mg/Kg, about 40 mg/Kg, about 45 mg/Kg, about 50 mg/Kg, or a range between any two of the preceding values.

A pharmaceutical composition of the present disclosure can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

A pharmaceutical composition of the present disclosure is manufactured in a manner which itself will be known in view of the instant disclosure, for example, by means of conventional mixing, granulating, dragee-making, dissolving, extrusion, or lyophilizing processes. Suitable formulations for parenteral administration include aqueous solutions of the active compound in a water-soluble form such as, for example, a water-soluble salt, alkaline solution, or acidic solution. Alternatively, a suspension of the active compound can be prepared as an oily suspension. Suitable lipophilic solvents or vehicles for such as suspension may include fatty oils (for example, sesame oil), synthetic fatty acid esters (for example, ethyl oleate), triglycerides, or a polyethylene glycol such as polyethylene glycol-400 (PEG-400). An aqueous suspension may contain one or more substances to increase the viscosity of the suspension, including, for example, sodium carboxymethyl cellulose, sorbitol, and/or dextran. The suspension may optionally contain stabilizers.

### Methods of Treatment

As described herein, the present disclosure provides a polypeptide-based adjuvant comprising a poly-lysine peptide conjugated to an amine containing building block according to the present disclosure or a polypeptide-based adjuvant that comprises the structure of Formula 1 according to the present disclosure, compositions thereof, and pharmaceutical compositions thereof, for use in treating a disease, disorder, condition or symptom thereof. For example, the polypeptide-based adjuvants, compositions, and pharmaceutical compositions described herein can be used for treating cancer, for inducing an immune response or enhancing an immune response, or for delivering therapeutic cargoes such as therapeutic agents, including but not limited to, small molecule compounds and polynucleotides. In aspects, the polypeptide-based adjuvants are cationic. In aspects, the polypeptide-based adjuvants are helical. In aspects, the polypeptide-based adjuvants are cationic and helical. In aspects, the polypeptide-based adjuvants are capable of inducing an immune response, e.g., an anti-tumor immune response

The polypeptide-based adjuvants for use as described herein comprise an amine containing building block, wherein the amine containing building block comprises a compound selected from the group consisting of substituted or unsubstituted heterocyclic compounds, substituted or unsubstituted heteroaryl compounds, linear or branched alkyl amines, substituted or unsubstituted aza-crown ethers, and combinations thereof. In some aspects, the amine containing building block comprises one or more of a C₁-C₆ alkyl, halogen, cyano, or hydroxy group. In aspects, the amine containing building block comprises a compound selected from the group consisting of piperazine, 1-methylpiperazine, 1-(2-hydroxyethyl)piperazine, 1-[2-(2-hydroxyethoxy)ethyl]piperazine, morpholine, piperidine, 1-aza-12-crown-4, 1-aza-15-crown-5,1-aza-18-crown-6, dimethyl amine, diethyl amine, dipropyl amine, dibutyl amine, trimethyl amine, triethyl amine, tripropyl amine, tributyl amine, 1-benziimidazole, and combinations thereof.

The polypeptide-based adjuvants for use described herein comprise a substituted oxybenzoyl-L-lysine. In aspects, the substituted oxybenzoyl-L-lysine is selected from the group consisting of 4-(2-chloroethyl)oxybenzoyl-L-lysine, 4-(3-chloropropyl)oxybenzoyl-L-lysine, 4-(4-chlorobutyl)oxybenzoyl-L-lysine, 4-(5-chloropentyl)oxybenzoyl-L-lysine, 4-(6-chlorohexyl)oxybenzoyl-L-lysine, 4-(7-chloroheptyl)oxybenzoyl-L-lysine, and 4-(8-chlorooctyl)oxybenzoyl-L-lysine.

In aspects of the polypeptide-based adjuvants for use described herein, the poly-lysine peptide has a molecular weight (g/mol) ranging from about 1,000 to about 500,000.

In aspects of the polypeptide-based adjuvants for use described herein, the poly-lysine peptide comprises one or more building blocks selected from the group consisting of immunogenic agents, therapeutic agents, imaging agents, stabilizing agents, targeting agents, linkers, and combinations thereof. In some aspects, the immunogenic agent is selected from the group consisting of piperazine, morpholine, piperadine, aza-crown ether, alkyl amine, benzimidazole, and combinations thereof. In some aspects, the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, and combinations thereof. In some aspects, the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof. In some aspects, the stabilizing agent is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof. In some aspects, the targeting agent is selected from the group consisting of antibodies, nanobodies, aptamers, small molecules, and combinations thereof. In some aspects, the targeting agent is conjugated to the polypeptide by a linker. In some aspects, the linker is selected from the group consisting of streptavidin-biotin, carboxylic acid-amines, disulfides, azide-alkynes, azide-DBCO, thiolenes, and combinations thereof.

In aspects of the polypeptide-based adjuvants for use described herein, the poly-lysine peptide is conjugated to or encapsulates a therapeutic agent. In some aspects, the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, and combinations thereof.

In aspects of the polypeptide-based adjuvants for use described herein, the poly-lysine peptide is conjugated to or encapsulates an imaging agent. In some aspects, the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof.

In aspects of the polypeptide-based adjuvants for use described herein, the polypeptide-based adjuvant is conjugated to a stabilizer. In some aspects, the stabilizer is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof.

The polypeptide-based adjuvants, compositions, and pharmaceutical compositions for use described herein can be administered by any means that achieves its intended purpose. For example, administration can be by the oral, parenteral, subcutaneous, intravenous, intramuscular, intraperitoneal, transdermal, intranasal, transmucosal, rectal, intravaginal or buccal route, or by inhalation. The dosage administered and route of administration will vary, depending upon the circumstances of the particular subject, and taking into account such factors as age, gender, health, and weight of the recipient, condition or disorder to be treated, kind of concurrent treatment, if any, frequency of treatment, and the nature of the effect desired.

The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, intraocular, intravitreal, periorbital, epidural and intrasternal injection and infusion, as well as in vivo electroporation. Non-parenteral routes include an oral, topical, epidermal or mucosal route of administration, for example, intranasally, vaginally, rectally, sublingually or topically. Administering can also be performed, for example, once, a plurality of times, and/or over one or more extended periods.

Cancer: As described herein, the present disclosure provides a polypeptide-based adjuvant comprising a poly-lysine peptide conjugated to an amine containing building block according to the present disclosure or a polypeptide-based adjuvant that comprises the structure of Formula 1 according to the present disclosure, compositions thereof, and pharmaceutical compositions thereof, for use in treating cancer in a subject in need thereof. Thus, the methods of the present disclosure include treating cancer in a subject in need thereof, comprising administering to the subject an effective amount of a composition or a pharmaceutical composition comprising a polypeptide-based adjuvant comprising a poly-lysine peptide conjugated to an amine containing building block according to the present disclosure or a polypeptide-based adjuvant that comprises the structure of Formula 1 according to the present disclosure. The methods of the present disclosure also include imaging a cancer in a subject in need thereof, comprising administering to the subject an effective amount of a composition or a pharmaceutical composition comprising a polypeptide-based adjuvant comprising a poly-lysine peptide conjugated to an amine containing building block according to the present disclosure or a polypeptide-based adjuvant that comprises the structure of Formula 1 according to the present disclosure, wherein the polypeptide-based adjuvant is conjugated to an imaging agent described herein.

The methods of the present disclosure also include a method for inducing an immune response or enhancing an immune response in a subject in need thereof, comprising administering to the subject an effective amount of a composition or a pharmaceutical composition comprising a polypeptide-based adjuvant comprising a poly-lysine peptide conjugated to an amine containing building block according to the present disclosure or a polypeptide-based adjuvant that comprises the structure of Formula 1 according to the present disclosure. In aspects, the subject has or is suffering from a cancer as described herein.

In some aspects, the cancer in the subject in need thereof is a tumor. In a further aspect, the tumor is a carcinoma. In some aspects, the tumor is a solid tumor. A “solid tumor” includes, but is not limited to, a sarcoma, melanoma, carcinoma, glioblastoma or other solid tumor including breast, prostate, lung, kidney, and pancreatic tumors. In some aspects, the cancer is selected from the group consisting of a breast cancer, a brain cancer, a prostate cancer, a lung cancer, and a colorectal cancer. In some aspects, the cancer is a breast cancer or a breast cancer metastases.

The term “sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzkycell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated according to the methods disclosed herein include, e.g., leukemia, Hodgkin's disease, non-Hodgkin's lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, pancreatic cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, peritoneal cancer, fallopian tube cancer, or uterine papillary serous carcinoma.

In some aspects, the cancer is relapsed, refractory, or refractory following at least one prior therapy comprising administration of at least one anti-cancer agent. The term “relapsed” refers to a situation where a subject, that has had a remission of cancer after a therapy, has a return of cancer cells. As used herein, the term “refractory” or “resistant” refers to a circumstance where a subject, even after intensive treatment, has residual cancer cells in the body. In some aspects, the cancer is metastatic.

In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, the cancer is selected from the group consisting of breast cancer, ovarian cancer, colon cancer, pancreatic cancer, and prostate cancer. In some aspects, the cancer is breast cancer.

A “cancer” or “cancer tissue” can include a tumor at various stages. In certain aspects, the cancer or tumor is stage 0, such that, e.g., the cancer or tumor is very early in development and has not metastasized. In some aspects, the cancer or tumor is stage I, such that, e.g., the cancer or tumor is relatively small in size, has not spread into nearby tissue, and has not metastasized. In other aspects, the cancer or tumor is stage II or stage III, such that, e.g., the cancer or tumor is larger than in stage 0 or stage I, and it has grown into neighboring tissues but it has not metastasized, except potentially to the lymph nodes. In other aspects, the cancer or tumor is stage IV, such that, e.g., the cancer or tumor has metastasized. Stage IV can also be referred to as advanced or metastatic cancer.

In aspects, the methods of treating cancer or inducing an immune response or enhancing an immune response disclosed herein, the poly-lysine peptide of the polypeptide-based adjuvant comprises one or more building blocks selected from the group consisting of immunogenic agents, therapeutic agents, imaging agents, stabilizing agents, targeting agents, linkers, and combinations thereof. In some aspects, the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, and combinations thereof. In some aspects, the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof. In some aspects, the stabilizing agent is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof. In some aspects, the targeting agent is selected from the group consisting of antibodies, nanobodies, aptamers, small molecules, and combinations thereof. In some aspects, the linker is selected from the group consisting of streptavidin-biotin, carboxylic acid-amines, disulfides, azide-alkynes, azide-DBCO, thiolenes, and combinations thereof.

In some aspects, the methods of treating cancer or inducing an immune response or enhancing an immune reponse disclosed herein further comprise (a) administering chemotherapy; (b) performing surgery; (c) administering radiation therapy; or (d) any combination thereof.

In other aspects, the methods of treating cancer or inducing an immune response or enhancing an immune reponse described herein can be combined with standard of care for cancer therapy. In some aspects, standard of care includes, but is not limited to, chemotherapy, radiotherapy, immunotherapy, targeted therapy, and combinations thereof.

In aspects, the methods of treating cancer or inducing an immune response or enhancing an immune reponse described herein further comprise administering a therapeutic agent selected from the group consisting of an anti-PD-1 inhibitor, an anti-PD-L1 inhibitor, an anti-CTLA4 inhibitor, an anti-CD47 inhibitor, an indole 2,3-dioxygenase inhibitor, and a combination thereof.

In aspects of the methods described herein, the polypeptide-based adjuvant comprising a poly-lysine peptide conjugated to an amine containing building block according to the present disclosure, compositions thereof, and pharmaceutical compositions thereof can be administered simultaneously or sequentially (before or after) with the administration of, e.g., the standard of care or the therapeutic agent described herein. In some aspects, the polypeptide-based adjuvant according to the present disclosure, compositions thereof, or pharmaceutical compositions thereof are administered about an hour, two hours, three hours, four hours, five hours, six hours, seven hours, eight hours, nine hours, ten hours, eleven hours, twelve hours, eighteen hours, twenty four hours, one day, two days, three days, four days, five days, six days, seven days, one week, two weeks, three weeks, four weeks, five weeks, six weeks, seven weeks, eight weeks, nine weeks, ten weeks, eleven weeks, twelve weeks, eighteen weeks, twenty four weeks, one month, two months, three months, four months, five months, six months, seven months, eight months, nine months, ten months, eleven months, or twelve months before the administration of, e.g., the standard of care or the therapeutic agent described herein. In some aspects, the polypeptide-based adjuvant according to the present disclosure, compositions thereof, or pharmaceutical compositions thereof are administered about an hour, two hours, three hours, four hours, five hours, six hours, seven hours, eight hours, nine hours, ten hours, eleven hours, twelve hours, eighteen hours, twenty four hours, one day, two days, three days, four days, five days, six days, seven days, one week, two weeks, three weeks, four weeks, five weeks, six weeks, seven weeks, eight weeks, nine weeks, ten weeks, eleven weeks, twelve weeks, eighteen weeks, twenty four weeks, one month, two months, three months, four months, five months, six months, seven months, eight months, nine months, ten months, eleven months, or twelve months after the administration of, e.g., the standard of care or the therapeutic agent described herein.

In some aspects of the methods described herein, the methods disclosed herein reduce the cancer burden. In some aspects of the methods described herein, the cancer burden is reduced by at least about 5%, at least about 10%, at least about 15%, at least about 20%, at least about 30%, at least about 40%, or about 50% as compared to the cancer burden prior to the administration of a polypeptide-based adjuvant described herein.

In some aspects of the methods described herein, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration of the polypeptide-based adjuvant described herein.

In some aspects, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of a polypeptide-based adjuvant described herein. The term “stable disease” refers to a diagnosis for the presence of a cancer, however the cancer has been treated and remains in a stable condition, i.e. one that that is not progressive, as determined, e.g., by imaging data and/or best clinical judgment. The term “progressive disease” refers to a diagnosis for the presence of a highly active state of a cancer, i.e., one that has not been treated and is not stable or has been treated and has not responded to therapy, or has been treated and active disease remains, as determined by imaging data and/or best clinical judgment.

“Stable disease” can encompass a (temporary) tumor shrinkage/reduction in tumor volume during the course of the treatment compared to the initial tumor volume at the start of the treatment (i.e. prior to treatment). In this context, “tumor shrinkage” can refer to a reduced volume of the tumor upon treatment compared to the initial volume at the start of (i.e. prior to) the treatment. A tumor volume of, for example, less than 100% (e.g., of from about 99% to about 66% of the initial volume at the start of the treatment) can represent a “stable disease.”

“Stable disease” can alternatively encompass a (temporary) tumor growth/increase in tumor volume during the course of the treatment compared to the initial tumor volume at the start of the treatment (i.e. prior to treatment). In this context, “tumor growth” can refer to an increased volume of the tumor upon treatment inhibitor compared to the initial volume at the start of (i.e. prior to) the treatment. A tumor volume of, for example, more than 100% (e.g. of from about 101% to about 135% of the initial volume, preferably of from about 101% to about 110% of the initial volume at the start of the treatment) can represent a “stable disease.”

The term “stable disease” can include the following aspects. For example, the tumor volume does, for example, either not shrink after treatment (i.e. tumor growth is halted) or it does, for example, shrink at the start of the treatment but does not continue to shrink until the tumor has disappeared (i.e. tumor growth is first reverted but, before the tumor has, for example, less than 65% of the initial volume, the tumor grows again.

In some aspects, the subject exhibits a partial response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the polypeptide-based adjuvant described herein.

In some aspects, the subject exhibits a complete response about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of a polypeptide-based adjuvant described herein.

The term “response” when used herein can refer to a “tumor shrinkage” or a reduction in the number of tumors, for example, when a cancer has metastasized. The term “response” can also be reflected in a “complete response” or “partial response” of the patients or the tumors. The term “complete response” as used herein can refer to the disappearance of all signs of cancer in response to a specific therapy disclosed herein. The term “complete response” and the term “complete remission” can be used interchangeably herein. For example, a “complete response” can be reflected in the continued shrinkage of the tumor (as shown in the appended example) until the tumor has disappeared. A tumor volume of, for example, 0% compared to the initial tumor volume (100%) at the start of (i.e. prior to) the treatment can represent a “complete response.”

Treatment with a polypeptide-based adjuvant described herein can result in a “partial response” (or partial remission; e.g. a decrease in the size of a tumor, or in the extent of cancer in the body, in response to the treatment). A “partial response” can encompass a (temporary) tumor shrinkage/reduction in tumor volume during the course of the treatment compared to the initial tumor volume at the start of the treatment (i.e. prior to treatment).

In some aspects, administering a polypeptide-based adjuvant described herein improves progression-free survival probability by at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, compared to the progression-free survival probability of a subject not receiving the treatment.

In some aspects, administering a polypeptide-based adjuvant described herein improves overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not receiving the treatment.

Immunization methods: As described herein, the present disclosure provides a polypeptide-based adjuvant comprising a poly-lysine peptide conjugated to an amine containing building block according to the present disclosure or a polypeptide-based adjuvant that comprises the structure of Formula 1 according to the present disclosure, compositions thereof, pharmaceutical compositions thereof, and vaccines thereof, for use in inducing an immune response or enhancing an immune response in a subject in need thereof. Thus, the methods of the present disclosure include inducing an immune response or enhancing an immune response in a subject in need thereof, comprising administering to the subject an effective amount of a composition, a pharmaceutical composition, or a vaccine comprising a polypeptide-based adjuvant comprising a poly-lysine peptide conjugated to an amine containing building block according to the present disclosure or a polypeptide-based adjuvant that comprises the structure of Formula 1 according to the present disclosure.

As also described herein, the present disclosure provides a polypeptide-based adjuvant comprising a poly-lysine peptide conjugated to an amine containing building block according to the present disclosure or a polypeptide-based adjuvant that comprises the structure of Formula 1 according to the present disclosure, compositions thereof, pharmaceutical compositions thereof, and vaccines thereof, for use in immunizing or conferring a protective immunity against a cancer in a subject in need thereof. Thus, the methods of the present disclosure include immunizing or conferring a protective immunity against a cancer in a subject in need thereof, comprising administering to the subject an effective amount of a composition, a pharmaceutical composition, or a vaccine comprising a polypeptide-based adjuvant comprising a poly-lysine peptide conjugated to an amine containing building block according to the present disclosure.

The polypeptide-based adjuvant described herein and compositions thereof, can be administered in the form of a vaccine composition, which may be prepared according to methods known to those skilled in the art. Usually, viruses, generally in freeze-dried form, are mixed with a pharmaceutically acceptable excipient, such as water or phosphate buffered saline, wetting or stabilizing agents. By “pharmaceutically acceptable excipient” is meant any solvent, dispersion medium, charge etc, which does not produce a side reaction, for example allergic, in humans or animals. The excipient is selected according to the chosen dosage form, method and route of administration. Preferably, the vaccine compositions are prepared in injectable form, and may correspond to liquid solutions, suspensions or emulsions. The compositions may in particular comprise an aqueous solution buffered to maintain a pH of between about 6 and 9 (as determined with a pH meter at room temperature).

The vaccine compositions as described herein may be administered according to any route usually used for vaccination, for example the parenteral route (especially intradermal, subcutaneous, or intramuscular). For example, in aspects described herein the vaccine compositions are injectable compositions administered subcutaneously in the region of the left deltoid and right deltoid.

### Therapeutic Payload Delivery:

As described herein, the present disclosure provides a polypeptide-based adjuvant comprising a poly-lysine peptide conjugated to an amine containing building block according to the present disclosure or a polypeptide-based adjuvant that comprises the structure of Formula 1 according to the present disclosure, compositions thereof, and pharmaceutical compositions thereof, for use in delivering, for example, a small molecule compound or a polynucleotide to a cell, for example, in a subject in need thereof. Thus, the methods of the present disclosure include delivering, for example, a small molecule compound or a polynucleotide to a cell, for example, in a subject in need thereof, comprising administering the polypeptide-based adjuvant comprising a poly-lysine peptide conjugated to an amine containing building block according to the present disclosure or a polypeptide-based adjuvant that comprises the structure of Formula 1 according to the present disclosure, a composition thereof, or a pharmaceutical composition thereof, to a cell, such as a cell in a subject in need thereof.

In some aspects, the polypeptide-based adjuvant is conjugated to or encapsulates the small molecule compound or a polynucleotide. In some aspects the polypeptide-based adjuvant is conjugated to a small molecule compound or a polynucleotide. In some aspects, the polypeptide-based adjuvant encapsulates a small molecule or polynucleotide via ionic interactions. In some aspects the small molecule or polynucleotide is an imaging agent, fluorescent marker, dye, pharmaceutical agent, metabolite, immunogenic agent, or radio-labeled nucleotide. In some aspects, the pharmaceutical agent is a therapeutic agent and/or cytotoxic agent.

In some aspects the small molecule or polynucleotide may be tumor antigens, CD4+ T cell epitopes, cytokines, chemotherapeutic agents, radionuclides, small molecule signaling inhibitors, photothermal antennas, monoclonal antibodies, immune risk signaling molecules, other immunotherapeutic drugs, enzymes, antibiotics, antiviral agents (especially protease inhibitors alone or in combination with nucleoside for treatment of HIV or hepatitis B or C), Antiparasitic drugs (helminths, protozoa), growth factors, growth inhibitors, hormones, hormone antagonists, antibodies and biologically active fragments thereof (including humanized antibodies, single chain antibodies, and chimeric antibodies), antigen formulations, peptide drugs, anti-inflammatory drugs, immunomodulators (Toll to activate the innate immune system), ligands that bind to receptor-like receptors (including but not limited to CpG oligonucleotides), molecules that recruit and optimize the adaptive immune system, cytotoxic T lymphocytes, natural killer cells, and helper T cells (including molecules that activate or upregulate the action of, and molecules that inactivate or downregulate suppressor or regulatory T cells), agents that promote nanolipogel uptake into cells (dendritic cells and others such asntigen presenting cells), nutraceuticals (such as vitamins), and oligonucleotides (DNA, plasmid DNA, RNA, antisense, aptamers, small interfering RNA (siRNA), messenger RNA (mRNA), small hairpin RNA (shRNA), ribozymes include external guide sequences, and triplex forming agents for ribonuclease P).

In some aspects, the small molecule or polynucleotide is an anti-cancer agent. anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-a or transforming growth factor-β inhibitors, and antibodies to the epidermal growth factor receptor such as panitumumab (VECTIBIX®) and cetuximab (ERBITUX®).

For example, in aspects, the small molecule or polynucleotide is doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, a lipopolysaccharide, mRNA, siRNA, shRNA, plasmid DNA, or CpG oligonucleotides.

In some aspects, the polypeptide-based adjuvant is conjugated to or encapsulates an immunogenic agent. In some aspects the polypeptide-based adjuvant is conjugated to an immunogenic agent. In some aspects, the polypeptide-based adjuvant encapsulates an immunogenic agent, for example, via ionic interactions. For example, in aspects, the immunogenic agent is substituted or unsubstituted piperazine, morpholine, piperadine, aza-crown ethers, linear or branched alkyl amines, substituted or unsubstituted benzimidazole.

In some aspects, the polypeptide-based adjuvant is conjugated to or encapsulates an imaging agent. In some aspects the polypeptide-based adjuvant is conjugated to an imaging agent. In some aspects, the polypeptide-based adjuvant encapsulates an imaging agent, for example, via ionic interactions. For example, in aspects, the imaging agent is iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, or ruthenium(II)-sonosensitizers.

In some aspects, the polypeptide-based adjuvant is conjugated to or encapsulates a targeting molecule. In some aspects the polypeptide-based adjuvant is conjugated to a targeting molecule. In some aspects, the polypeptide-based adjuvant encapsulates a targeting molecule, for example, via ionic interactions. For example, in aspects, the targeting molecule is an antibody, nanobody, aptamer, or a small molecule.

The methods of delivery disclosed herein typically include using a composition, pharmaceutical composition, or vaccine loaded with one or more active agents, to deliver the one or more active agents into a cell, or to a cell's microenvironment. The methods typically include contacting the active agent-loaded composition, pharmaceutical composition, or vaccine with one more cells. In some aspects the method of delivery includes contacting the active agent-loaded composition, pharmaceutical composition, or vaccine with one more cells in vivo. In some aspects the method of delivery includes contacting the active agent-loaded composition, pharmaceutical composition, or vaccine with one more cells in vitro. In some aspects the method of delivery includes contacting the active agent-loaded composition, pharmaceutical composition, or vaccine with one more cells ex vivo.

In some aspects the small molecule or polynucleotide is delivered to the subject as a pharmaceutical composition. The subject can be, e.g., a mammal (e.g., a human) or any other subject described herein.

In some aspects, administering a polypeptide-based adjuvant as described herein is capable of loading therapeutic payloads by forming cleavable covalent bonds or using physical interactions and/or improving overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not receiving the treatment.

In some aspects, administering a polypeptide-based adjuvant as described herein, compositions thereof, or pharmaceutical compositions thereof, is administered according to any of the methods described herein. For example, in aspects, the polypeptide-based adjuvant as described herein, compositions thereof, or pharmaceutical compositions thereof is administered orally, intravenously, intraperitoneally, intratumorally, intramuscularly, subcutaneously, or intrathecally.

### Vaccines

As described herein, the present disclosure provides polypeptide-based adjuvants which may be used in the form of a cancer vaccine. Thus, the present disclosure provides a vaccine comprising a polypeptide-based adjuvant comprising a poly-lysine peptide conjugated to an amine containing building block according to the present disclosure, or a polypeptide-based adjuvant that comprises the structure of Formula 1 according to the present disclosure, and a pharmaceutically acceptable carrier or diluent suitable for use in a vaccine.

The vaccines as described herein include, for example, a whole-cell vaccine, a DNA vaccine, a peptide-based vaccine, and a protein-based vaccine. The vaccines as described herein are capable of eliciting an immune response against a cancer disclosed herein. For example, in some aspects, the cancer is selected from the group consisting of a breast cancer, a brain cancer, a prostate cancer, a lung cancer, and a colorectal cancer. In some aspects, the cancer is a breast cancer or a breast cancer metastases. In some aspects, the cancer can include, but is not limited to, adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment.

In some aspects, administering a polypeptide-based adjuvant described herein is capable of loading vaccines including peptide, mRNA or DNA vaccines and/or improving overall survival probability by at least about 25%, at least about 50%, at least about 75%, at least about 100%, at least about 125%, at least about 150%, at least about 175%, at least about 200%, at least about 225%, at least about 250%, at least about 275%, at least about 300%, at least about 325%, at least about 350%, or at least about 375%, compared to the overall survival probability of a subject not receiving the treatment.

In some aspects, the vaccines described herein further comprise an immunogenic compound. In some aspects, the immunogenic compound is selected from the group consisting of piperazine, morpholine, piperadine, aza-crown ether, alkyl amine, benzimidazole, and combinations thereof.

### Kits and Methods of Making

As described herein, the present disclosure provides kits comprising (i) a polypeptide-based adjuvant comprising a poly-lysine peptide conjugated to an amine containing building block according to the present disclosure or a polypeptide-based adjuvant that comprises the structure of Formula 1 according to the present disclosure; (ii) a composition thereof, (iii) a pharmaceutical composition thereof, or (iv) a vaccine thereof. In some aspects described herein, the kit further comprises instructions for use.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The polypeptide-based adjuvants described herein, compositions thereof, pharmaceutical compositions thereof, or vaccines thereof, may also be formulated into a syringeable composition. In which case, the container means may itself be a syringe, pipette, and/or other such like apparatus, from which the formulation may be applied to an area of the body, injected into the subject, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

As described herein, the present disclosure provides provides methods of making polypeptide-based adjuvant comprising a poly-lysine peptide conjugated to an amine containing building block according to the present disclosure or a polypeptide-based adjuvant that comprises the structure of Formula 1 according to the present disclosure, compositions thereof, pharmaceutical compositions thereof, or vaccines thereof.

In some aspects, the present disclosure provides, a method of making a polypeptide-based adjuvant described herein, a composition thereof, a pharmaceutical composition thereof, or a vaccine thereof, comprising: (a) making a poly-lysine peptide by synthesizing a lysine monomer followed by polymerization to form a poly-lysine peptide, and (b) conjugating an amine containing building block. In some aspects, the method further comprises (c) adding the obtained polypeptide-based adjuvant to a composition as described herein. In some aspects, the method further comprises (c) adding the obtained polypeptide-based adjuvant to a pharmaceutical composition as described herein. In some aspects, the method further comprises (c) adding the obtained polypeptide-based adjuvant to an anionic polymer in varying weight ratios to form a nanoparticle. Further aspects related to methods of making a polypeptide-based adjuvant described herein, a composition thereof, a pharmaceutical composition thereof, or a vaccine thereof, will be known to those of skill in the art and are illustrated at both high level and granular levels of specificity in the following Examples.

### EXAMPLES

### Example 1: Materials and Methods

The present example describes methods used to generate data described herein.

### Cell Lines

E0771, TUBO, 4T1, and SK-BR3 breast cancer cell lines were purchased from ATCC, and maintained in Dulbecco's minimum essential medium (DMEM) supplemented with 10% fetal bovine solution (FBS), 1% sodium pyruvate, 1% penicillin/streptomycin, and 0.2% normocin. The 4T 1 cell line was transfected with lentiviral encoding firefly luciferase gene to establish 4T1-Luc. 4T1-BR4-Luc were developed after four rounds of intracardiac injection/brain cell culture.

### Macrophage Preparation

Bone marrow-derived macrophages (BMDMs) were prepared from the femur and tibia of 6- to 10-week-old C57BL/6 bred at MD Anderson Cancer Center or The Jackson Laboratory, Balb/C (The Jackson Laboratory, USA), MyD88 KO mice (The Jackson Laboratory, USA) and tmem173 mice (The Jackson Laboratory, USA), by the previously established protocol. BMDMs were maintained in DMEM supplemented with a 30% L929 cell-conditioned medium (Stony Brook Cell Culture/Hybridoma facility), 20% FBS, 1% sodium pyruvate, and 1% penicillin/streptomycin. M1 macrophages and M2 macrophages were prepared by exposing BMDMs to lipopolysaccharide (LPS) (100 ng/mL) for 2 days or IL-4 (10 ng/mL) for 2 days. For THP-1 macrophage differentiation, THP-1 cells were treated with 200 nM phorbol 12-myristate 13-acetate for 3 days and then incubated without phorbol 12-myristate 13-acetate for another 2 days to obtain human macrophage-like cells.

### Reverse Transcription Quantitative Polymerase Chain Reaction (RT-qPCR)

M2 macrophages (1×10⁷ cells/100 mm² petri dish) were treated with polypeptides (4 μg/mL), valinomycin (1 μM), monensin (1 μM), or LPS (100 ng/mL) for 24 h; or M2 BMDMs were treated with P1 (4 μg/mL), SD (12 μg/mL), SDP1 (16 μg/mL), or LPS. RNA samples were prepared according to the manufacturer's guidelines (PureLink RNA Mini Kit, ThermoFisher Scientific, USA). 1 μg of total RNA was reverse-transcribed into complementary DNA (High Capacity cDNA Reverse Transcription Kit, ThermoFisher Scientific, USA). RT-qPCR was performed with a TaqMan™ Fast Advanced PCR master Mix (ThermoFisher Scientific, USA) with the corresponding primer (7500 FAST Real-time PCR System, Applied Biosystems, USA). Each ΔCt value was obtained by Ct_{target}−Ct_{GAPDH}. Each −ΔΔCt was calculated by ΔCt_{cont}−ΔCt_{target}. All the relative mRNA levels were quantified by 2^{-ΔΔCt}.

### Phagocytosis Assay

Phagocytosis was assessed by co-culturing eFluor 450-labeled cancer cells (eBioscience, USA) and carboxyfluorescein succinimidyl ester-labelled M2 macrophages (ThermoFisher Scientific, USA) as follows. Cancer cells (1.5×10⁵ cells/12-well plate) were plated and allowed to adhere for 1 day. Then macrophages (4.5×10⁵ cells) were added and co-cultured with the cancer cells for 1 day under the various treatment conditions. To assay phagocytosis inhibition, macrophages were pre-incubated with cyclosporin A (CsA; 10 μM) for 30 min or sodium 4-phenylbutyric acid (PBA; 5 mM) for 1 day. The cells were then isolated and stained with Sytox Red to exclude the dead cells. The degree of phagocytosis (%) was evaluated by using flow cytometry.

### Antigen Presentation Assay

E0771-cOVA cells were prepared as described previously. M2 BMDMs were co-cultured with E0771-cOVA cells and treated with P1, SD, SDP1, or LPS for 24 h. Cells were stained with the corresponding antibodies and Sytox Red to exclude dead cells. The mean fluorescence intensity of SIINFEKL-H2Kb on the M2 BMDMs was evaluated by flow cytometry.

### Confocal Laser Scanning Microscopy for Phagocytosis and Antigen Presentation

To visualize phagocytosis, cancer cells pre-stained with eFluor 670 were seeded on confocal chamber slides (4 Chamber Slide Systems, Lab-Tek II, USA) and then M2 macrophages stained with carboxyfluorescein succinimidyl ester (CSFE) were added to the chamber with P1, SD, SDP1, or LPS. The cells were incubated for 6 h and then fixed with 4% para-formaldehyde solution. For antigen presentation, E0771-cOVA cells pre-seeded on the chamber were co-cultured with M2 BMDMs, and the cells were then treated with P1, SD, SDP1, or LPS for 1 day. The cells were fixed and then incubated with anti-SIINFEKL-H2Kb (Biolegend, USA) for another day. The chamber slides were mounted, and the cell images were obtained by confocal microscopy (FV3000, Olympus, Japan).

### Western Blotting

M2 BMDMs (1.5×10⁷ cells/100 mm² petri dish) or cancer cells (2×10⁶ cells per 6-well plates) were treated with the experimental solutions. To evaluate inhibitory conditions, cells were pre-treated with CsA (10 μM, TCI America) for 3 h, or co-treated with 4-PBA (5 mM in phosphate-buffered saline [PBS], Tocris Bioscience, USA) and chloroquine (10 μg/mL, Alfa Aesar, USA). Each protein was extracted by using RIPA buffer containing 1% protease inhibitor and 1% phosphatase inhibitor cocktail. Protein concentrations were adjusted to 3 mg/mL by using a BCA kit, with FBS used as a standard. Electrophoresis was carried out by loading proteins (40 μg) into each lane of a sodium dodecyl sulfate-polyacrylamide gel (SDS-PAGE) gel, after which the proteins were transferred to a polyvinylidene fluoride membrane. After blocking, the PVDF membrane was treated with the corresponding primary antibody, and then washed with TBS-T three times before the treatment with the secondary antibody. The blot signals were visualized with a Chemidoc system (ChemiDoc MP imaging system, BioRad, USA).

### Mitochondrial ROS Level

M2 macrophages (5×10⁶ cells/12 well plate) were treated with P1, SD, SDP1, LPS, or cGAMP for 1 day before adding MitoSOX Red (2.5 μM final concentration, ThermoFisher Scientific, USA) for 10 min. The cells were rinsed with PBS three times, collected with a scraper, and stained with antibodies and cell viability dye to measure mitochondrial ROS level by flow cytometry.

mtDNA Release Assay

M2 macrophages (1.5×10⁷ cells/100 mm³ dish) were treated with P1, SD, SDP1, LPS, or cGAMP for 1 day. The cytosol fraction was obtained with a Mitochondrial/Cytosol Fractionation Kit (abcam, UK) before DNA was isolated with a DNeasy Blood & Tissue Kit (QIAGEN, USA). RT-qPCR was used to measure cytosolic mtDNA contents. The copy number of cytosolic mtDNA encoding ND-1 was normalized to that of total mtDNA.

### Flow Cytometry for Extracellular Staining

BMDMs (6×10⁵ cells/well in a 12 well-plate) were treated with polypeptides (4 μg/mL), valinomycin (0.25 μM), monensin (0.25 μM), or LPS (100 ng/mL) for 24 h; M2 BMDMs were co-cultured with cancer cells and treated with P1, SD, SDP1, or LPS for 24 h. The cells were blocked with anti-mouse antiCD-16/32 (1:1000 in FACs buffer, Biolegend USA) and then stained with surface maker-staining antibodies for 30 min. After washing with FACs buffer, the cells were dyed with Sytox Red (1:4000 in FACs Buffer, ThermoFisher Scientific, USA) for 15 min to exclude the dead cells. Marker expression was evaluated by flow cytometry (FACs Celesta, BD Biosciences, USA). CD11b+F4/80+Sytox Red-cells were gated for live macrophages.

### Confocal Laser Scanning Microscopy (CLSM) for Staining Organelles

M2 BMDMs seeded on confocal dishes were dyed with Mitotracker Deep Red (400 nM, ThermoFisher Scientific, USA), ER tracker Red (100 nM, ThermoFisher Scientific, USA), or Lysotracker Deep Red (50 nM, ThermoFisher Scientific, USA) for 30 min before being treated with FITC-P1 and FITC-SDP1 for 3 h (mitochondria) and for 30 min, 1 h, and 3 h (lysosomes). The cells were washed with PBS three times and then fixed with a 4% paraformaldehyde solution to visualize the cells by CLSM.

### Immunofluorescence

M2 BMDMs seeded on confocal dishes were treated with P1, SD, SDP1, LPS, or cGAMP for 1 day or with FITC-P1 and FITC-SDP1 for 3 h for STING IF. The cells were washed with PBS three times, fixed and permeabilized to stain intracellular proteins before the cells were treated with the primary antibody overnight at 4° C. and then with the fluorophore-tagged secondary antibody for 1 h. Cells were imaged by CLSM.

### Lysosomal pH

M2 macrophages (5×10⁴ cells/96 well plate) were treated with P1, SD, SDP1, LPS, or cGAMP for 1 day and then stained with LysoSensor Yellow/Blue DND-160 (1:1000 diluted, ThermoFisher Scientific, USA) for 5 min. After washing with PBS three times, the fluorescence intensity was measured at Ex-329/Em-440 and Ex-360/Em-550 with a fluorescence plate reader (CLARIOstar Plus, BMG Labtech, USA). The standard curve of lysosomal pH value was determined with a intracellular pH calibration buffer kit (ThermoFisher Scientific, USA).

### Cell Membrane Destabilization

M2 BMDMs (5×10⁵ cells/12-well plate) were treated with calcein (1 μg/mL) and then treated with P1, SD, SDP1, LPS, or cGAMP for 3 h. The cells were rinsed with PBS three times, stained with cell viability dye, and then fixed with a 4% paraformaldehyde solution before being stained with antibodies. The fluorescence intensity of calcein was quantified by flow cytometry.

### Enzyme-Linked Immunosorbent Assay for Cytokines

For supernatant collection in vitro, cancer cells were co-cultured with M2 macrophages in different treatment conditions for 1 day. Cytokines (TNF-α, IL-1β, IL-2, and IFN-γ) were quantified with mouse or human ELISA kits according to the manufacturer's procedures.

### T Cell Activation and Proliferation

T cells were harvested from the spleens of OT-I or OT-II transgenic mice and isolated per the manufacturer's guideline (EasySep Mouse T cell Isolation Kit, STEMCELL Technologies, USA). M2 macrophages co-cultured with cancer cells were treated with P1, SD, SDP1, or LPS for 1 day before the addition of CSFE-stained OT-I cells or OT-II cells. The cells were incubated for 3 days to allow T cell proliferation. To study T cell activation, macrophages were isolated from co-culture conditions by using CD45-positive selection and then further co-cultured with OT-I or OT-II T cells for 3 days. The cells were isolated and then stained with fluorophore-tagged antibodies and cell viability dye. Flow cytometry was used to evaluate naïve T cells (CD44−CD62L+), effector T cells (CD44+CD62L−) and memory T cells (CD44+CD62L+). The Invitrogen CellTrace CFSE kit was used to monitor distinct generations of proliferating cells by dye dilution.

### Mice

Six- to ten-week-old C57BL/6 (bred at MD Anderson Cancer Center), Balb/C (The Jackson Laboratory, USA), MyD88 knockout (The Jackson Laboratory, USA) and tmem173gt (The Jackson Laboratory, USA) mice were maintained at the animal facility of The University of Texas MD Anderson Cancer Center in specific-pathogen-free environments. All animal use was approved by the Institutional Animal Care and Use Committee of MD Anderson Cancer Center, and all experiments complied with approved protocols and institutional policies.

### Tumor Models

Orthotopic breast tumor models were established by implanting E0771 (1.5×10⁶ cells) or 4T1 (5×10⁵ cells) breast cancer cells into a mammary pad in mice. Tumors were measured with calipers, and tumor volumes were calculated as ellipsoids (π/6×length×width). Mice with tumors of similar size were randomly sorted into experimental groups on day 10 after tumor-cell inoculation. Treatments (P1 10 mg/Kg for EO771 tumor, P1 20 mg/Kg for 4T1 tumor, tail-vein injection) took place on day 10, 12, and 14 for EO771 tumors, and on day 8, 10, and 12 for 4T1 tumors. For combined therapy, αPD1 (200 g, intraperitoneal injection, BioXcell, USA) treatment was given on days 11, 13, and 15 for either E0771 or 4T1 tumors. Mice were euthanized with CO₂ if tumor volumes exceeded 1000 mm³ or ulcerations (>5 mm in diameter) developed.

### Flow Cytometry In Vivo

Tumors and spleens were excised from tumor-bearing mice 3 days after the last treatment. Tumors were dissociated into single-cell suspensions by using a mouse Tumor Dissociation Kit (Miltenyi Biotech). CD45+ cells were enriched by using a CD45− positive selection method (CD45 Microbeads mouse, Miltenyi Biotech, USA). Splenocytes were obtained by grinding the spleens and then lysing red blood cells. Both types of cells were stained with the corresponding antibodies for analysis by flow cytometry.

### Hemolysis Assay

Red blood cells were obtained by centrifuging blood samples from C57BL6 mice, and then treated with HEPES, P1, SDP1, or 10% Triton X-100 for 2 h at 37° C. The red blood cells were then spun down to collect supernatant. The absorbance (A) at 540 nm of each sample was detected with a multi-reader. Hemolysis (%) was calculated as:

Hemolysis(%)=(Aₛₐₘₚₗₑ−APBS)/(Atriton X-100−APBS)×100(%).

### Evaluation of Systemic Toxicity

Six-week-old female C57BL/6J mice were intravenously injected with HEPES, P1, SDP1, or cGAMP on day 0, 2, and 4. Body weights of the mice in each group were measured every other day. On day 16, blood samples were collected from each mouse via cardiac puncture, and ALT, AST, BUN, and creatinine levels were evaluated from blood plasma and blood cells were used to assess the population of leukocytes (CD45+ cells).

### Immunohistochemical Assays

Tumor tissues were excised 2 or 3 days after the final treatment, fixed with 4% paraformaldehyde, embedded in paraffin, and sliced into 5-μm sections. Tumor sections were then deparaffinized and stained with the corresponding primary antibodies, incubated overnight, and then stained with fluorophore-labeled secondary antibodies for 1 h. Nuclei were stained with 4′,6-diamidino-2-phenylindole (DAPI; 1 g/mL, Milipore Merck, USA), and immunohistologic images were obtained by confocal laser scanning microscopy.

### RT-qPCR In Vivo

Tumors excised 1 day after the final treatment were dissociated into single-cell suspensions by using a mouse Tumor Dissociation Kit (Miltenyi Biotech). F4/80+ cells (for macrophages) and CD11c+ cells (for dendritic cells) were isolated by using F4/80 and CD11c magnetic beads (Miltenyi Biotech, USA), respectively. RNAs from tumor-infiltrating APCs were extracted according to the manufacturer's guidelines with a PureLink RNA Mini Kit (ThermoFisher Scientific, USA). RT-qPCR was conducted as described earlier.

### Tumor Metastasis Model

The spontaneous metastatic breast tumor model was established by injecting 4T1-BR4-mcherry Luc cells (1.5×106 cells/50 μL) into the mammary pad, after which tumors were resected 14 days later. Treatments were implemented on day 16, 18, and 20 for P1 and cGAMP (20 mg/kg, intravenous injection) or day 17, 19, and 21 for αPD1 (200 g, intraperitoneal injection). Spontaneous 4T1 metastases were visualized with an IVIS spectrum imaging system (IVIS Lumina XR, Perkin Elmer).

### Statistical Analysis

All the data are shown as means standard deviation (S.D.) from at least triplicate conditions unless otherwise indicated. Statistical analyses involved unpaired Student's t-test for two groups or one way ANOVA for multiple comparisons. Survival was assessed by the Kaplan-Meier method and compared by the log-rank (Mantel-Cox) test. The p value of less than 0.05 were regarded as statistical significance. Statistical analyses were conducted with Graph Prism 9 and Microsoft Excel 2020. No animal was excluded from the analyses. Quantitative analyses of confocal laser scanning microscopy images were conducted using Image J.

### Example 2: P1, P2, and P3 Synthesis

### Synthesis of 4-(5-hydroxypentyloxy)benzoic acid

Methyl 4-hydroxybenzoate (6 g, 39.4 mmol) were dissolved in anhydrous DMF (50 mL) with 5-bromopentan-1-ol (4.73 mL, 47.3 mmol) and potassium carbonate (10.9 g, 78.9 mmol). The mixture was stirred at 100° C. for overnight. The suspension was diluted with deionized (DI) water (200 mL) and then the product was extracted with ethyl acetate (50 mL) three times. To get rid of the remaining water in the organic phase, anhydrous magnesium sulfate was added in the ethyl acetate solution. The solvent was removed using a rotary evaporator to isolate methyl 4-(5-hydorxypentyloxy)benzoate. Desterification of methyl 4-(5-hydorxypentyloxy)benzoate was proceeded at 100° C. for overnight using 3 N NaOH solution (200 mL). The cloudy solution was obtained by adding 33 wt % HCl solution until the white precipitation was observed. The white powder was washed with pre-warmed DI water five times to remove the unreacted species before drying the product in the oven (6.4 g, 28.5 mmol).

### Synthesis of 4-(5-chloropentyloxy)benzoyl chloride

4-(3-hydroxypropoxy)benzoic acid (5 g, 22.3 mmol) was suspended in thionyl chloride (40 mL) with a catalytic amount of DMF (200 μL). The reaction was proceeded at 80° C. until the suspension became completely clear. The excessive thionyl choride and HCl were evaporated at RT under reduced pressure to obtain 4-(5-chloropentyloxy)benzoyl chloride. 4-(5-chloropentyloxy)benzoyl chloride was immediately used after the preparation.

### Synthesis of 4-(5-chloropentyloxy)benzoyl-L-lysine

4-(5-chloropentyloxy)benzoyl chloride (5 g, 25 mmol) dissolved in tetrahydrofuran (THF, 10 mL) was added in copper (II) lysinate solution (2.87 g lysine dissolved, 19.6 mmol) with sodium bicarbonate (7 g, 51.0 mmol). The benzoylation was carried out at RT for overnight. The purple precipitate was isolated by centrifugation, and sequentially washed with THE and deionized water three times, respectively. In order to remove copper, the precipitate was washed with saturated ethylenediaminetetraacetic acid disodium salt (EDTA-2Na) solution until the precipitate became white. The white powder was lyophilized to obtain 5-(4-chloropentyloxy)benzoyl-L-lysine (4.3 g, 11.6 mmol).

### Synthesis of 4-(5-chloropentyloxy)benzoyl-L-lysine N-carboxyanhydride (NCA)

In a glove box, 4-(5-chloropentyloxyl)benzoyl-L-lysine (1 g, 2.7 mmol) was suspended in anhydrous THE (30 mL) with triphosgene (0.4 g, 1.35 mmol). The reaction mixture was stirred at 70° C. until the suspension became totally transparent. The solution was diluted with excessive EtOAc (100 mL), and the organic phase was washed with saturated NaHCO₃ solution and deionized water three times, respectively, before the addition of anhydrous MgSO4. The solution was concentrated using a rotary evaporator, and then precipitated with n-hexane (200 mL). The skin color power was isolated by vacuum filtration, and dried under the reduced pressure to obtain 4-(5-chloropentyloxyl)benzoyl-L-lysine NCA (0.97 g, 2.44 mmol).

### NCA Polymerization

In a glove box, 4-(5-chloropentyloxyl)benzoyl-L-lysine NCA (0.8 g, 2.01 mmol) was solubilized in anhydrous DMF (10 mL). The feeding ratio of monomer to initiator (hexamethyldisilazane) was 40. The monomers were polymerized at RT for 2 days. The viscous DMF solution was precipitated with DI water, and then washed with DI water three times. Poly (4-(5-chloropentyloxy)benzoyl-L-lysine) was obtained after lypophilization (0.65 g).

### Synthesis of P1, P2, and P3

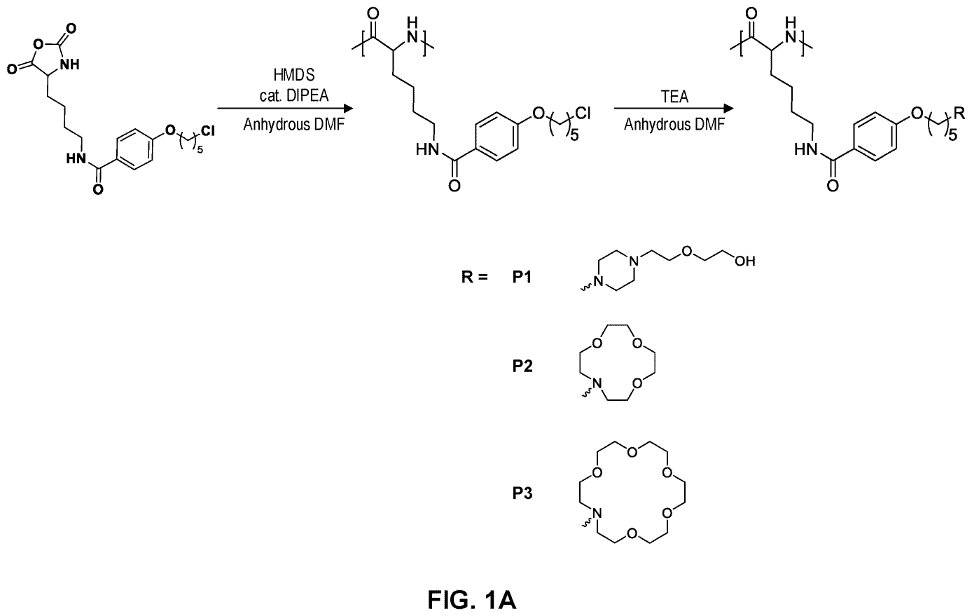

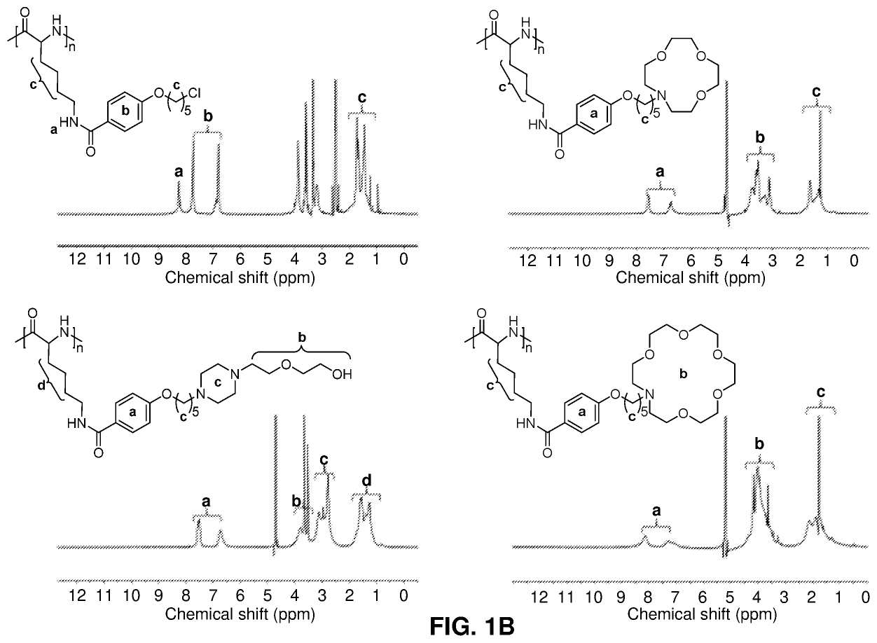

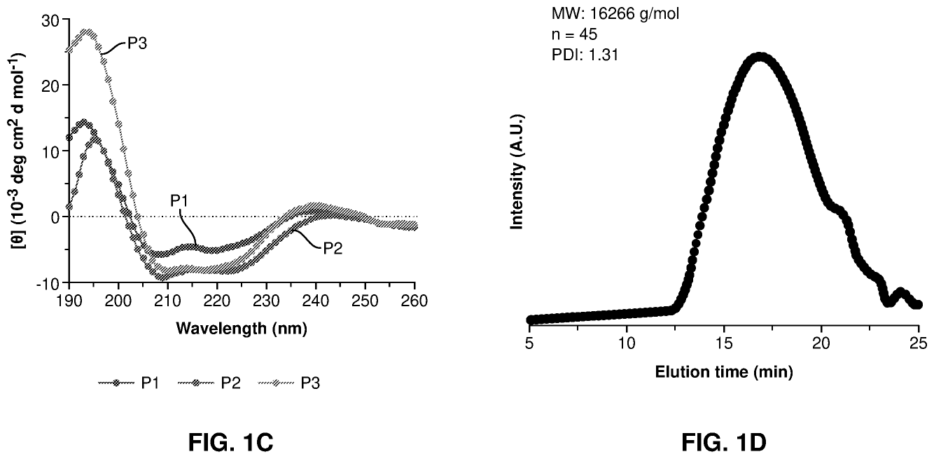

Poly-(4-(5-chloropentyloxy)benzoyl-L-lysine)) (0.08 g) was dissolved in anhydrous dimethylformamide (DMF; 10 mL) with triethylamine (296 μL, 2.27 mmol) before the addition of R (1-hydroxyethylethoxypiperazine; 0.16 g, 0.91 mmol, TCI, Japan, 1-aza-12-crown-4; 0.16 g, 0.91 mmol, TCI, Japan 1-aza-18-crown-6; 0.20 g, 0.91 mmol, TCI, Japan). The reaction was allowed to proceed at 100° C. for 1 day. The reaction mixture was precipitated with saturated Na₂CO₃ solution. The solid was washed with deionized water three times before each polypeptide was solubilized in 0.1 N HCl. The solution was dialyzed against deionized water to remove the excessive HCl and then lyophilized to obtain P1 (0.13 g), P2 (0.12 g) and P3 (0.14 g) (FIG. 1A).

### Synthesis of succinylated dextran

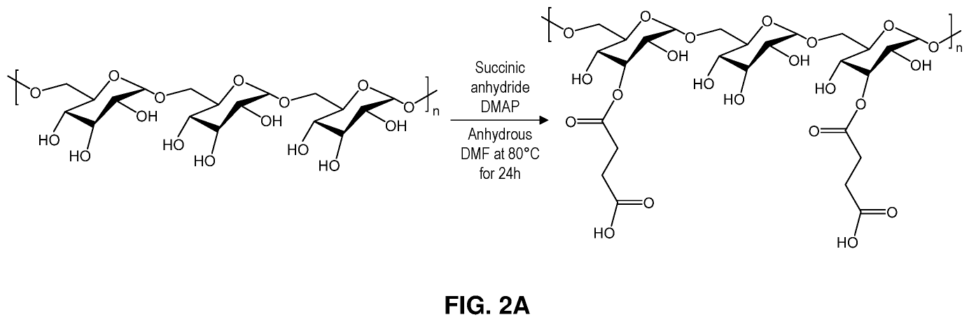

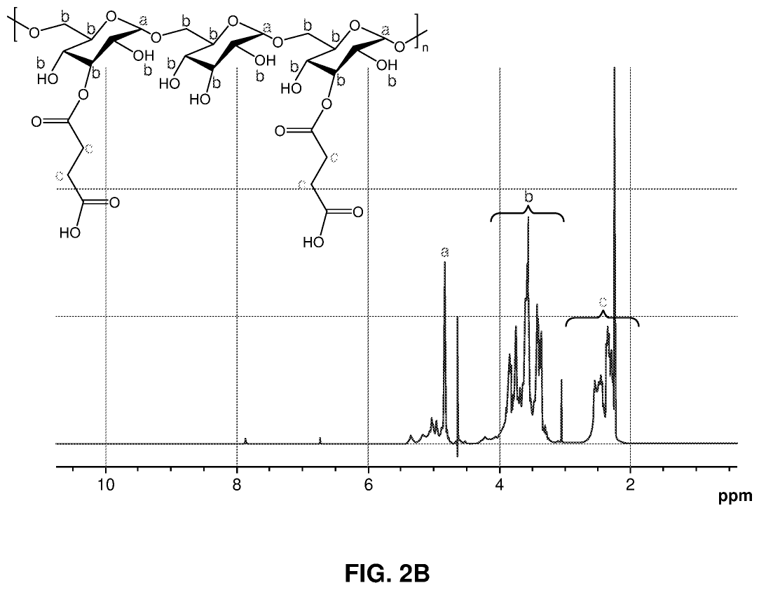

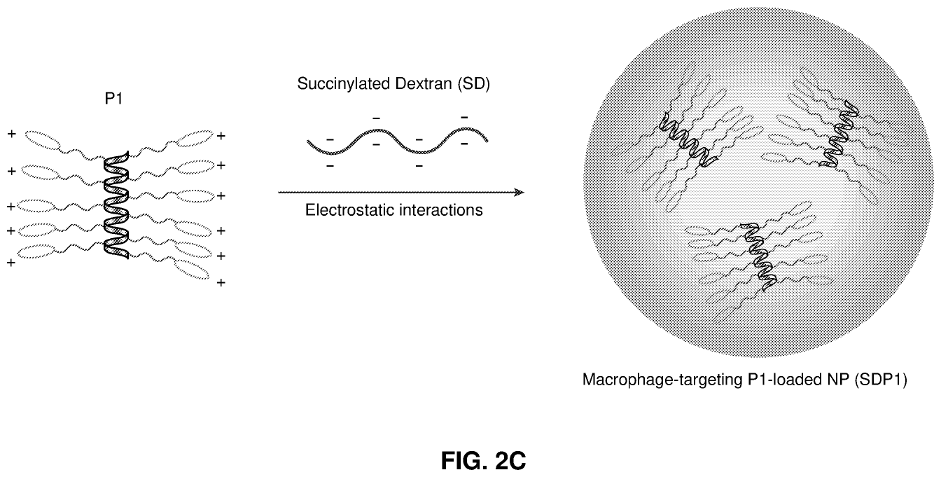

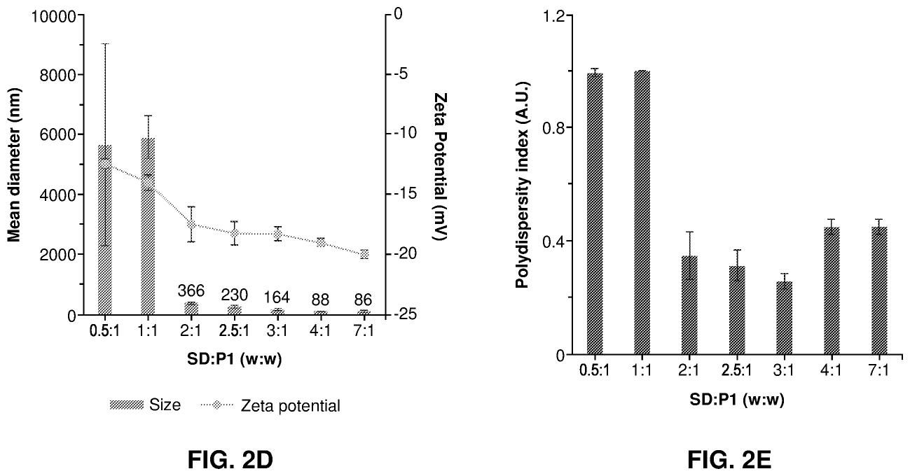

Succinylated dextran was prepared by adding dextran (1 g, 0.025 mmol) suspended in anhydrous DMF (25 mL) to dimethylaminopyridine (0.38 g, 3 mmol, 0.5 equivalent of the mers) and succinic anhydride (1.26 g, 12.5 mmol, 2 equivalent of the mers). The mixture was agitated at 80° C. for 24 h. To remove the unreacted reagents, the suspensions were poured into acetone (200 mL). The brownish precipitate was washed with acetone three times and then solubilized in saturated sodium carbonate solution to ionize the carboxylic acid groups. After the removal of insolubilized dextran by centrifugation, the brownish solution was dialyzed against deionized water for 1 day and then freeze-dried to obtained succinylated dextran (FIG. 2A).

### Formation of Nanoparticles

Nanoparticles (NPs) between P1 and SD were prepared by using electrostatic attractions. P1 (10 mg/mL in HEPES buffer; 10 mM HEPES, 150 mM NaCl) was added to SD (10 mg/mL in HEPES buffer) by varying different weight ratios, and then the solution was mixed by pipetting for 30 seconds. The solution was diluted to obtain a final concentration of 40 μg/mL. All of the mean diameters, polydispersity indices, and zeta potentials were determined by dynamic light scattering and electrophoretic methods (FIG. 2C).

### Example 3: PI Stimulates Innate Immune Sensors

[Macrophages (5×10⁵ cells/well in a 12 well-plate) were treated with polypeptides P1, P2, and P3 (4 μg/mL), valinomycin (1 μM), and monensin (1 μM) for 24 h before the addition of dichloro-dihydro-fluorescein diacetate (DCFH-DA, 2.5 μM final concentration, Sigma Aldrich, USA) for 30 min. To evaluate intracellular ROS levels, M2 BMDMs were treated with P1, SD, SDP1, or LPS for 1 day. The cells were isolated, and stained with the antibodies for 30 min. After washing with flow cytometry staining (FACs) buffer and then adding FACS buffer with Sytox Red to exclude the dead cells, intracellular ROS levels were measured by flow cytometry as described in Example 1. CD1 lb+F4/80+ cells were gated for BMDMs. Relative intracellular ROS level was quantified by Fₛₐₘₚₗₑ/F_{cont} (F: fluorescence intensity).

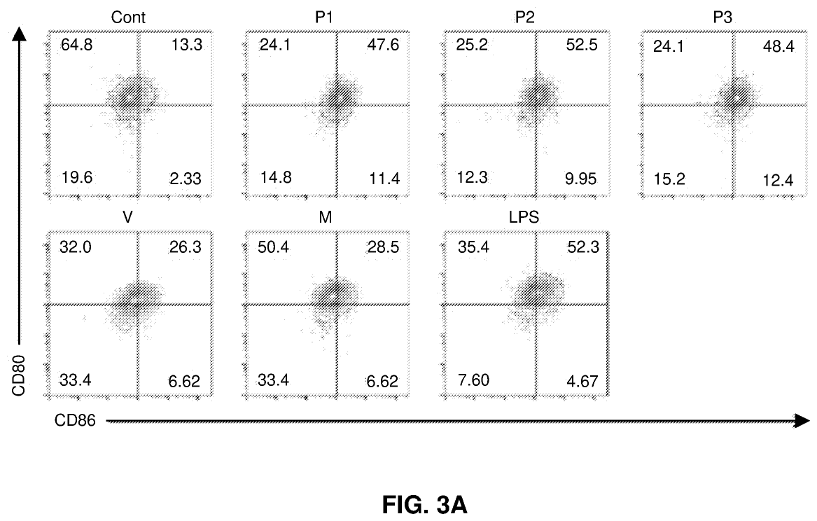

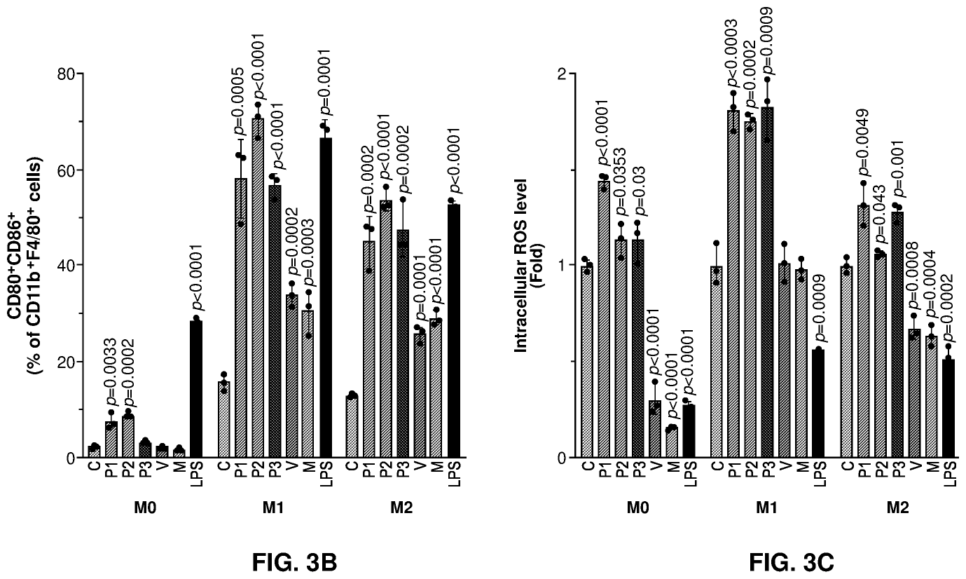

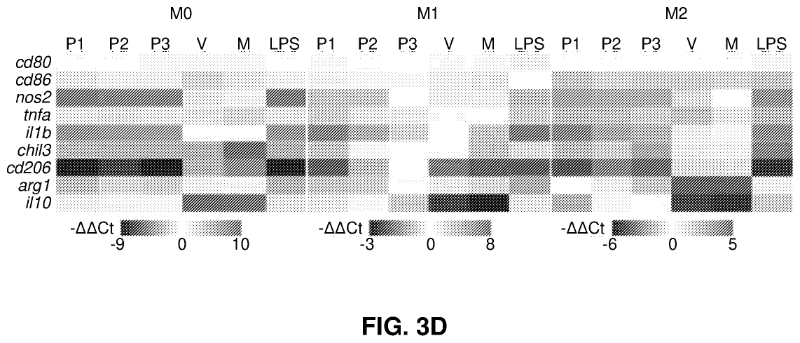

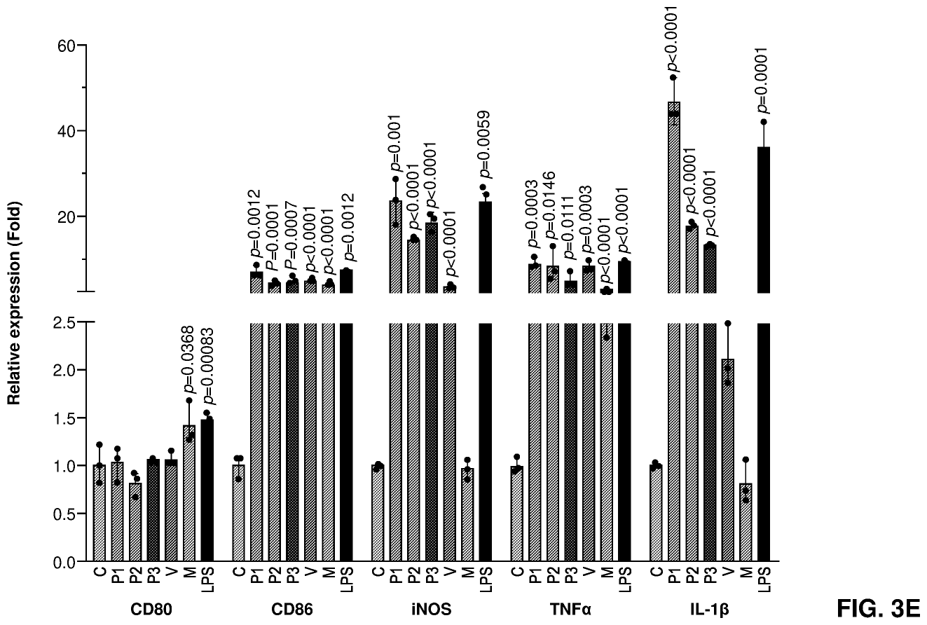

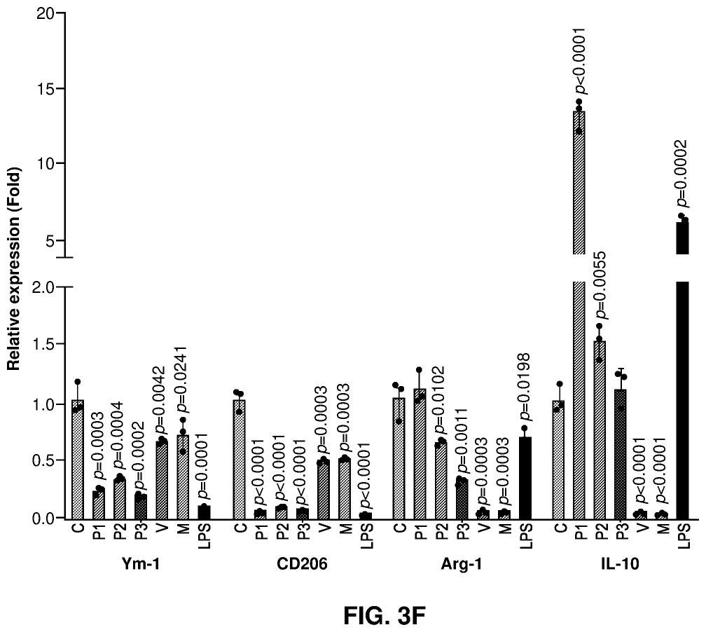

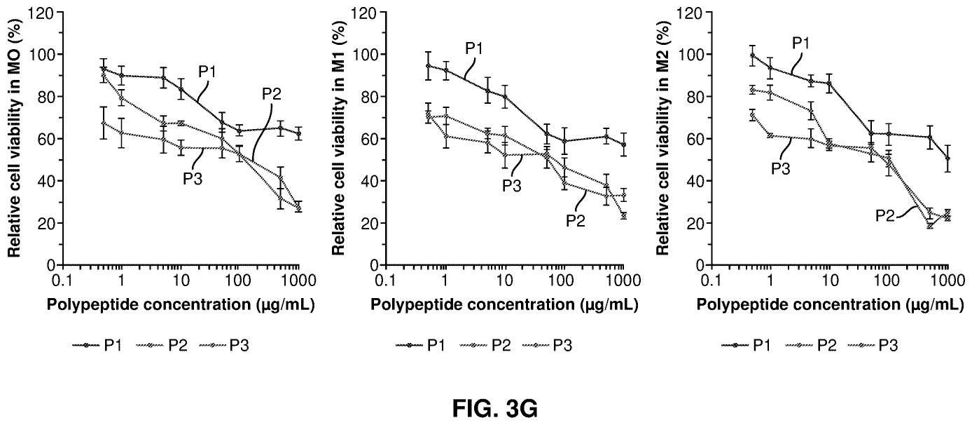

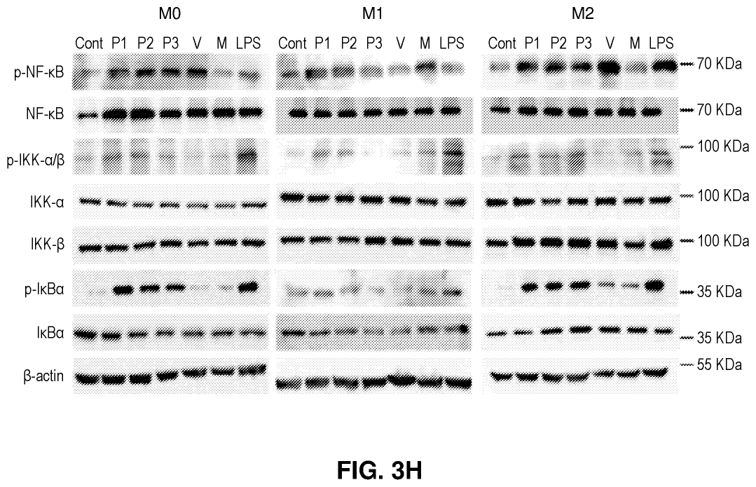

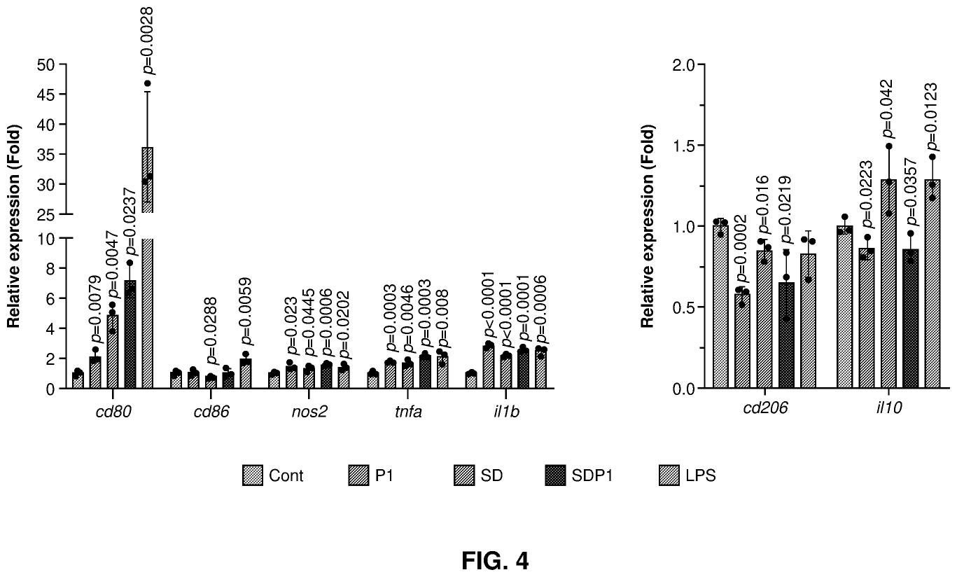

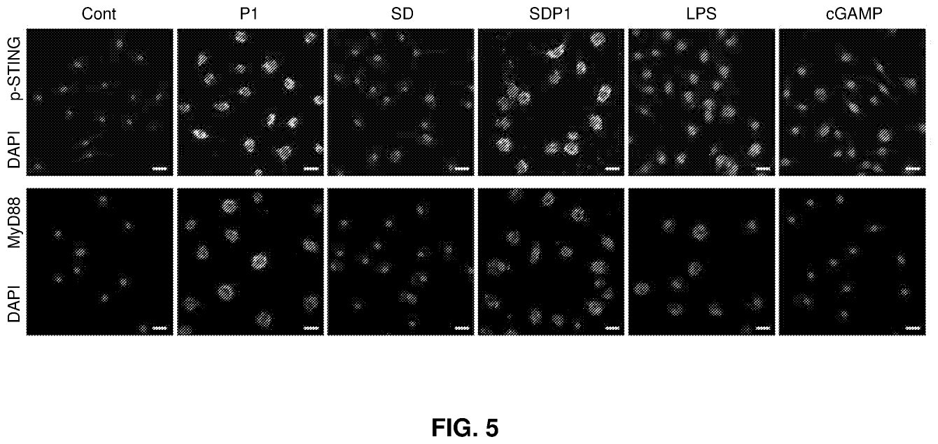

All poly peptides (P1-P3) polarized the macrophage phenotype from M2 to M1. P1 was selected as an optimal cationic polypeptide-based immune activator, which was associated with higher upregulation of pro-inflammatory signaling without toxicity as compared to P2 and P3 as shown in FIGS. 3A-G.

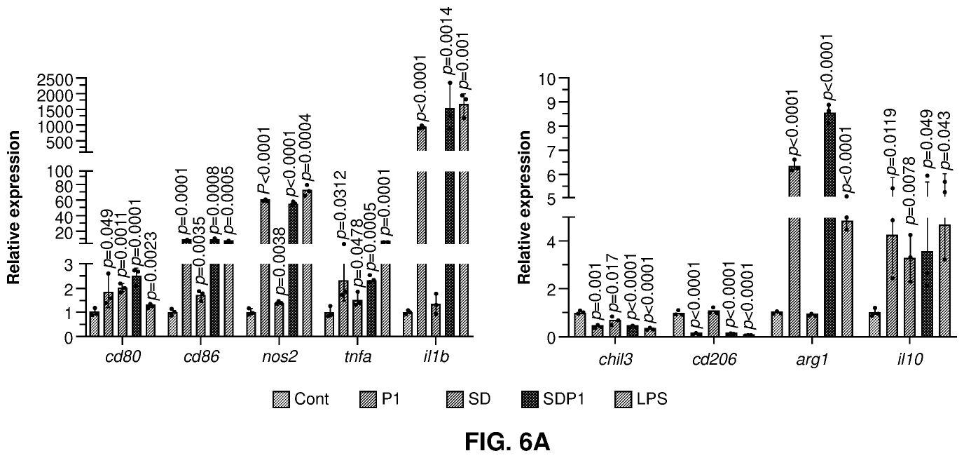

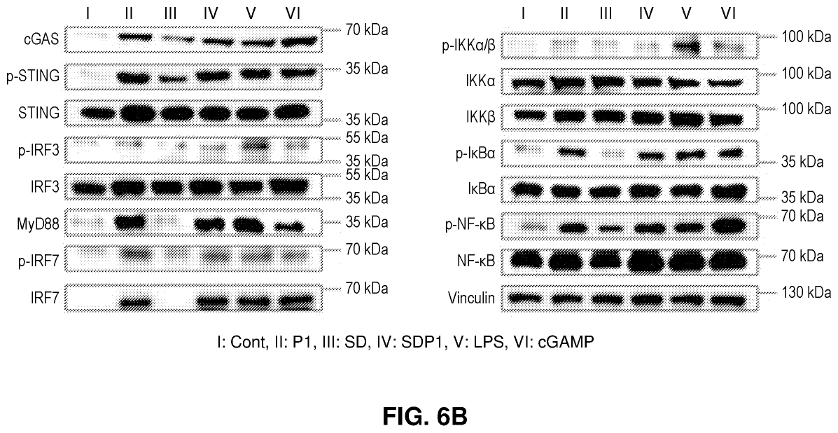

FIG. 6A shows P1 and SDP1 upregulated M1-related genes cd80, cd86, nos2, tnfa and il1b significantly compared with untreated M2 BMDMs. M2-related cd206, and chil3, were reduced after a 24-hour treatment with P1 or SDP1. P1 and SDP1 also increased the expression of M1-associated genes cd80, nos2, tnfa, and il1b while reducing that of M2-relevant cd206 and il10 in human THP-1 macrophages.

A western blot was performed as described in Example 1, to assess whether the cationic polypeptides activate innate immune sensors in APCs. As shown in FIG. 6B, P1 and SDP1 treatments resulted in increased phosphorylation of STING and its downstream effectors interferon regulatory factor 3 (IRF3) and IRF7. In addition, these polypeptides also increased the expression of MyD88, a universal adapter protein for TLR signaling that is responsible for activating the transcriptional factor nuclear factor κ-light-chain-enhancer of activated B cells (NF-κB). Increased phosphorylation of the canonical NF-κB member p65 and upregulation of multiple pro-inflammatory genes was also observed. Taken together, these findings suggest that the cationic polypeptides exerted its pro-inflammatory effects on APCs via multiple independent pathways.

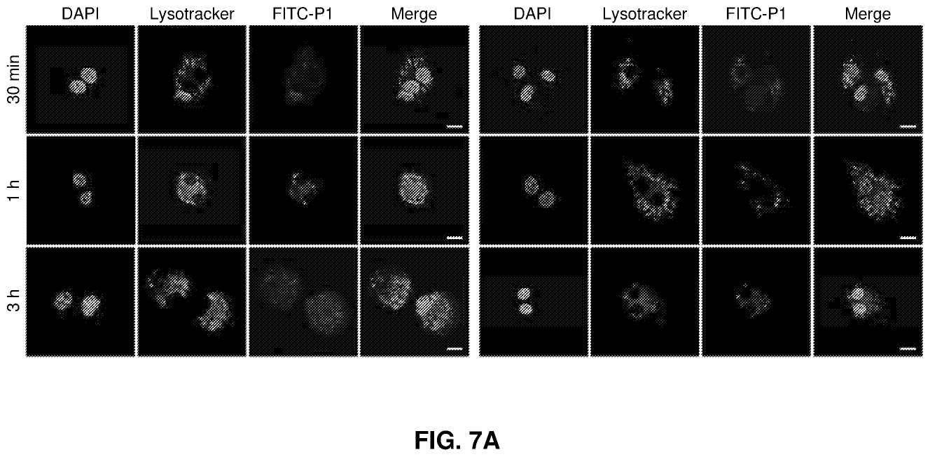

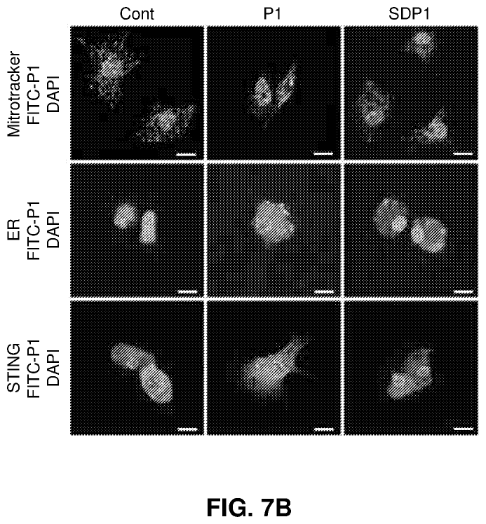

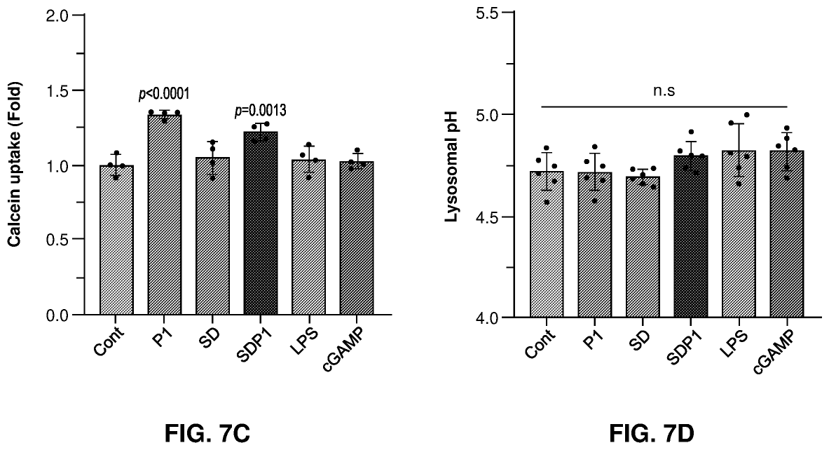

To identify the cellular compartment in which the polypeptides stimulate innate immune sensors, P1 and SDP1 were fluorescently labeled and imaged by CLSM in an immunofluorescence assay as described in Example 1. Fluorescently labelled P1 and SDP1 was initially retained in lysosomes, but ultimately trafficked to the endoplasmic reticulum (ER), and not observed in mitochondria (FIG. 7A and FIG. 7B). Since P1 and SDP1 destabilizing the macrophage plasma or lysosome membranes was not observed, it is unlikely that innate immune sensors were activated by loss of integrity of the extracellular-cytosolic barriers within the APCs. (FIG. 6C and FIG. 6D).

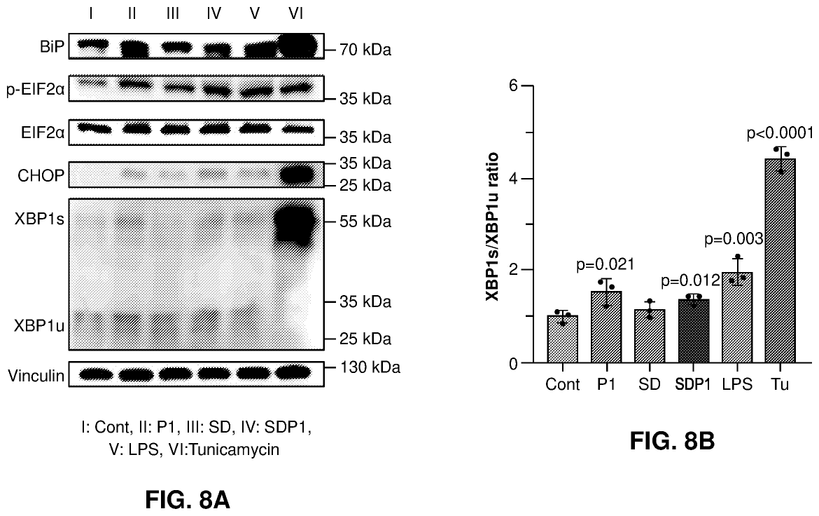

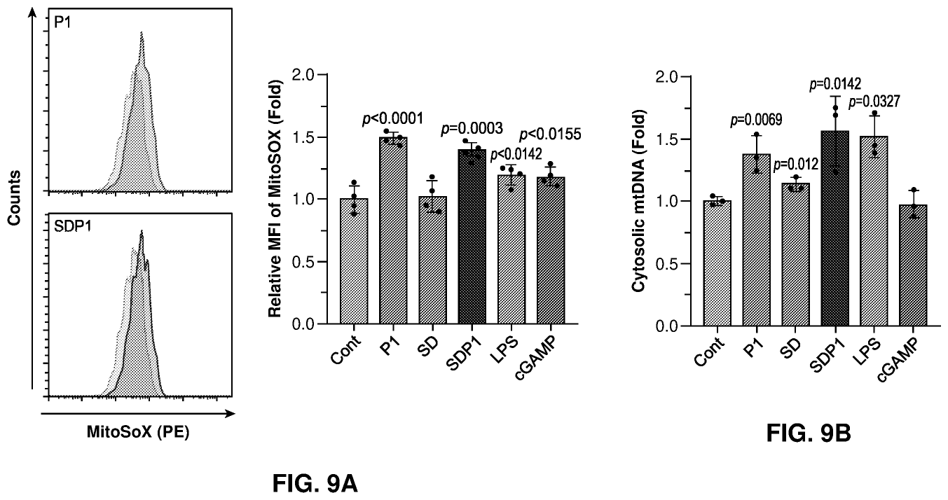

Further, it was observed that the ER stress markers including binding immunoglobulin protein (BiP), eukaryotic translation initiation factor 2α (EIF2α), C/EBP homologous (CHOP), X-box-binding protein 1 (XBP1) were upregulated following polypeptide treatments but not to the detrimental levels seen with tunicamycin, a robust inducer of ER stress (FIG. 8A and FIG. 8B).

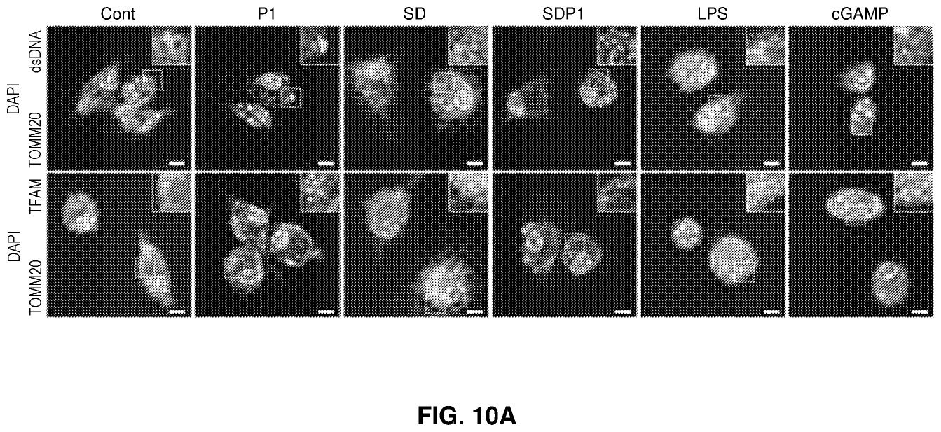

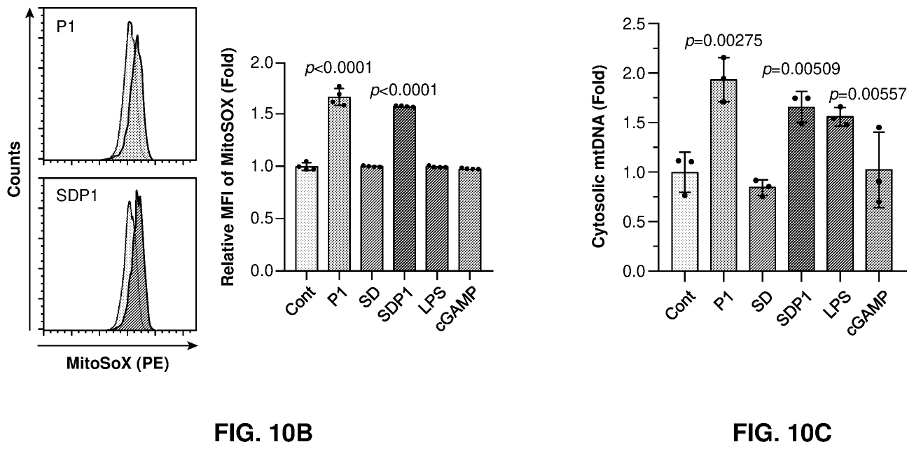

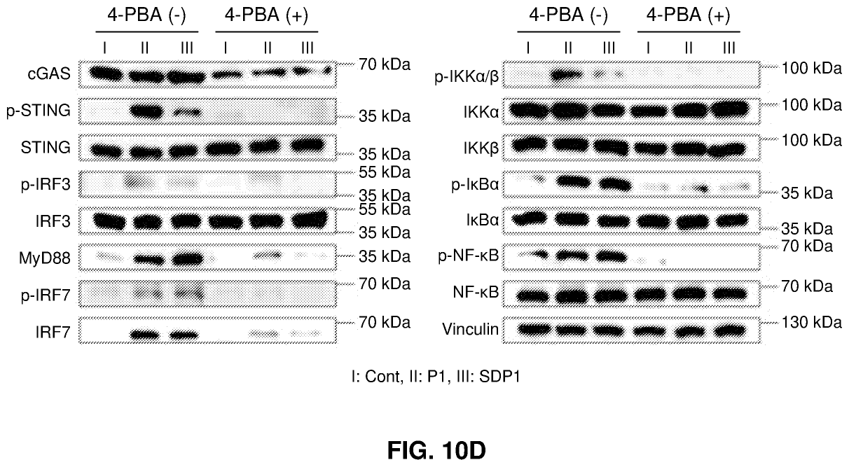

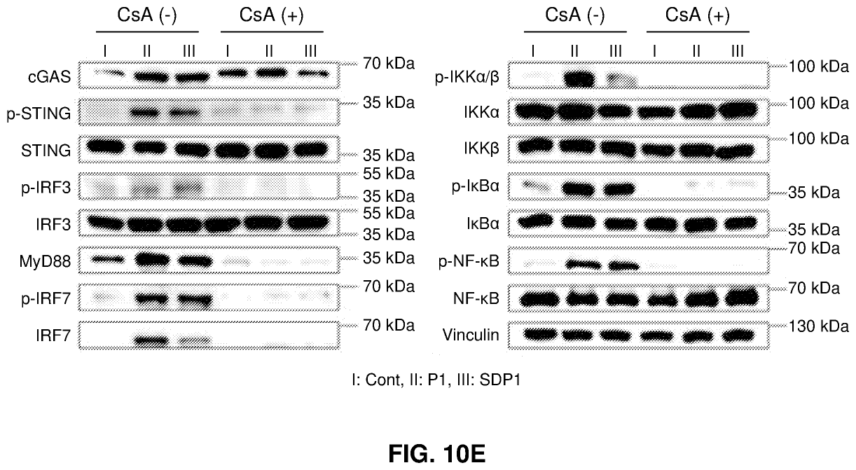

As shown in FIGS. 10A-C, the upregulation of ER stress markers is associated with the accelerated release of mitochondrial DNA (mtDNA) and mitochondrial transcription factor A (TFAM) from treated macrophages.

To determine whether ER stress-mediated mtDNA release is necessary for the cationic polypeptide induced immune sensor activation, western blotting of proteins related to TLR pathways was performed according to Example 1.

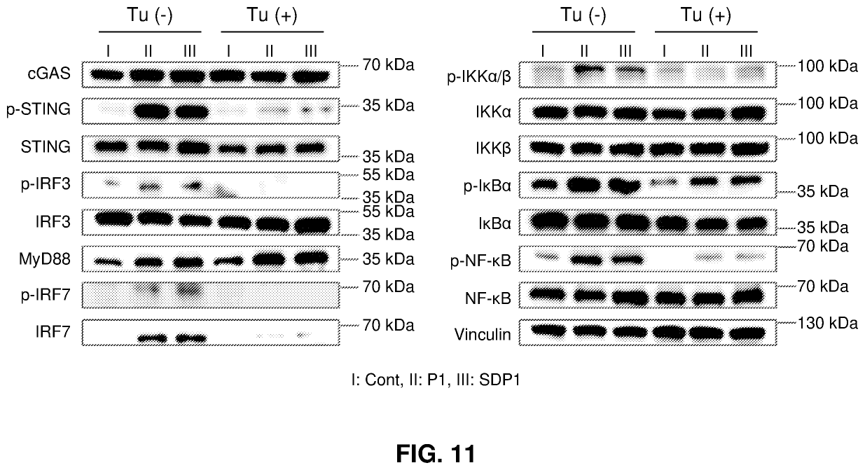

FIG. 10D and FIG. 10E show that inhibition of ER stress or mtDNA release by sodium 4-phenylbutyric acid (4-PBA) or cyclosporin A (CsA), respectively, abrogated the activation of STING, MyD88, and canonical NF-κB pathways, confirming that ER stress-mediated mtDNA release is essential for cationic polypeptide-induced immune sensor activation. By contrast, excessive ER stress induction with tunicamycin, which are known to promote immunogenic cell death, inhibited activation of MyD88 and STING in macrophages, thus confirming that the molecular design of P1 facilitated generation of mild ER stress to propagate pro-inflammatory signaling within a physiologic therapeutic window (FIG. 11).

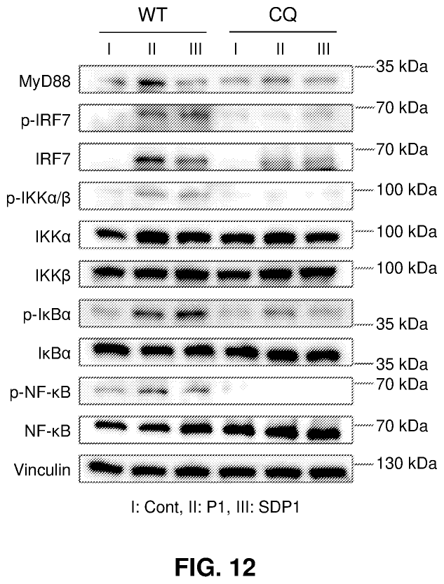

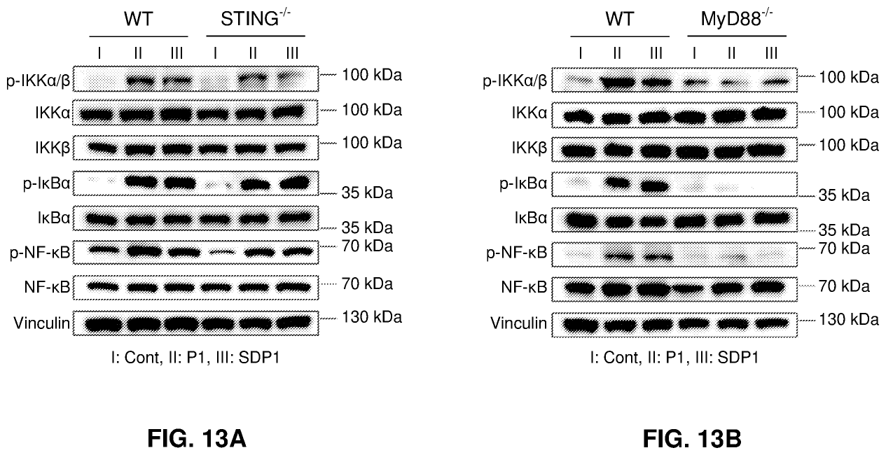

To examine how P1-induced mtDNA facilitates TLR activation, western blotting of proteins related to TLR pathways was carried out under autophagy-inhibiting conditions as described above. Inhibition of autophagy by chloroquine was found to deactivate the MyD88 and canonical NF-κB pathways, implying that cationic polypeptide-induced mtDNA release promoted autophagy and subsequent TLR activation (FIG. 12). P1-mediated activation of canonical NF-κB pathways by using STING−/− or MyD88−/−BMDMs. P1 and SDP1 treatment resulted in the stimulation of canonical NF-κB pathways in STING−/−BMDMs but not in MyD88 KO−/−BMDMs, implying that P1 promoted pro-inflammatory NF-κB signaling primarily via the MyD88 signaling pathway (FIG. 13A and FIG. 13B).

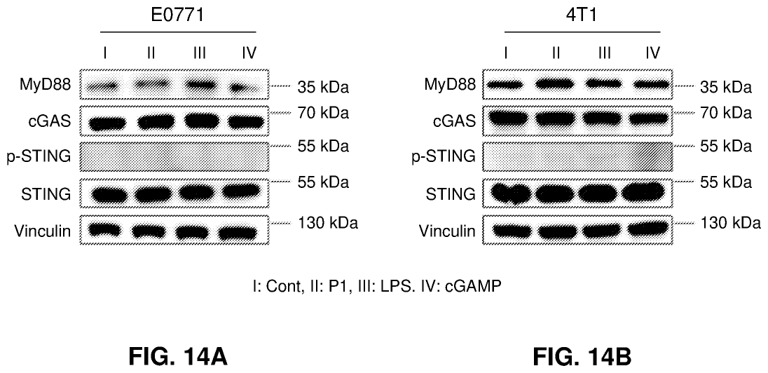

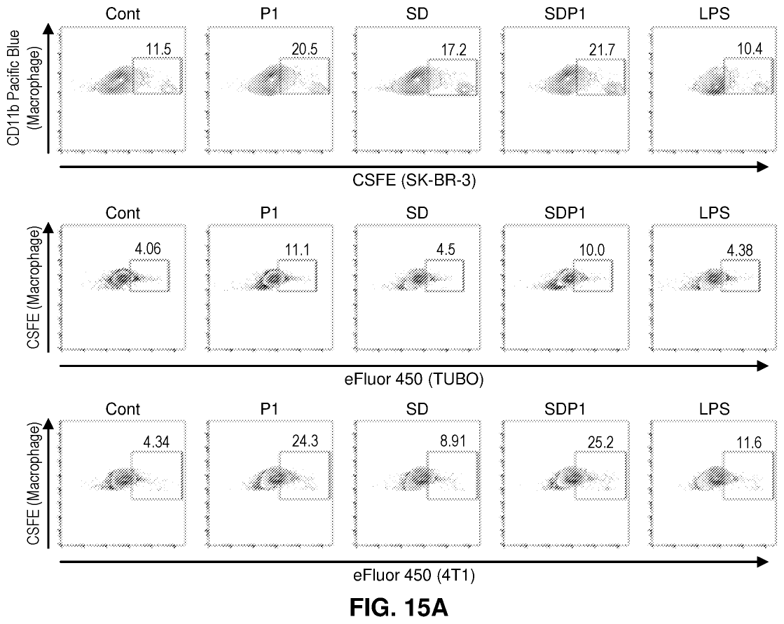

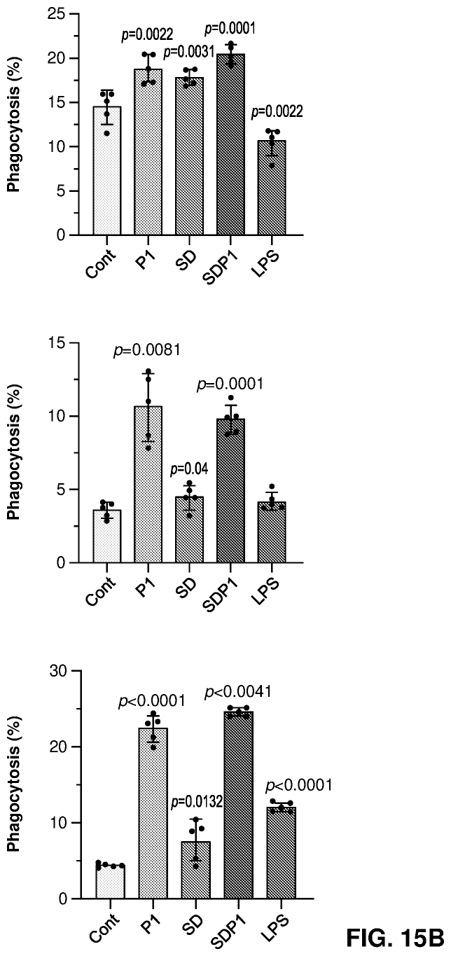

To examine whether P1 affects the activation of innate immune sensors in cancer cells, a western blotting of MyD88 and STING pathways in E0771 and 4T1 breast cancer cells was performed (FIG. 14A and FIG. 14B). P1 treatment did not alter MyD88 or p-STING expression. Collectively, these findings indicate that treatment with P1 generates pro-inflammatory responses selectively in APCs by activating STING and TLRs.

### Example 4: P1 Enhances the Phagocytosis of Cancer Cells and the Cross-Priming of Antigen-Specific T Cells Achieved by Professional Antigen-Presenting Cells

M2-polarized mouse macrophages co-cultured with murine breast cancer cells were treated with P1, SDP1, SD, and LPS to confirm that treatment with cationic polypeptides could enhance effector functions of APCs. Visualization of phagocytosis in cancer cells was performed according to Example 1.

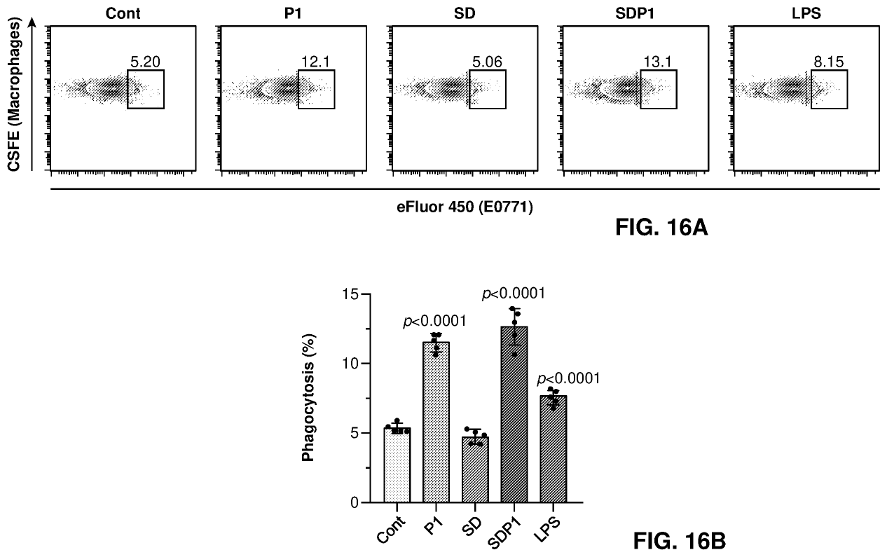

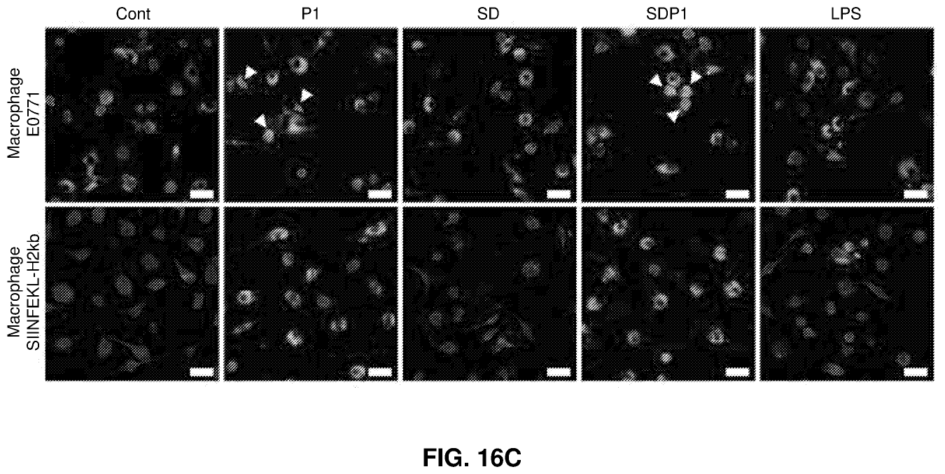

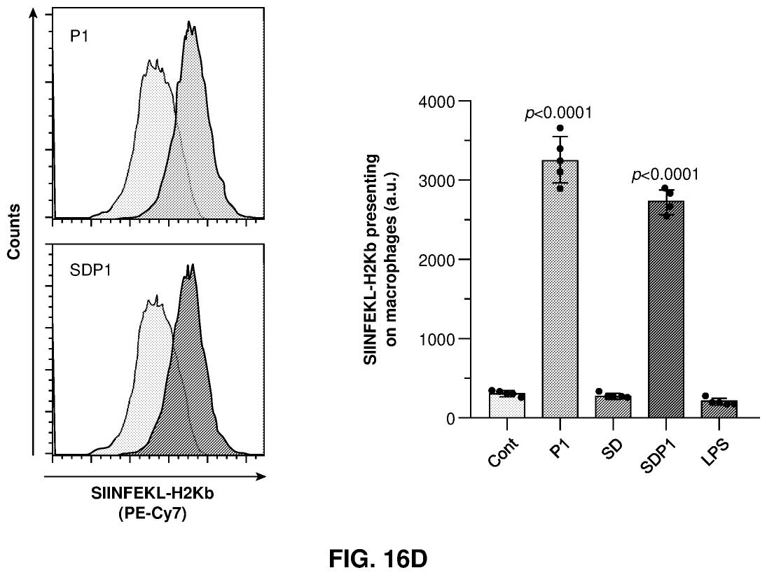

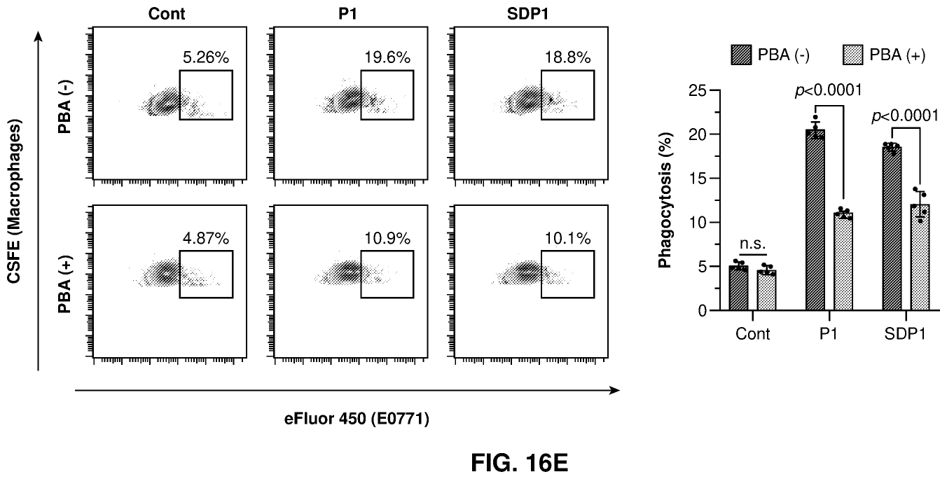

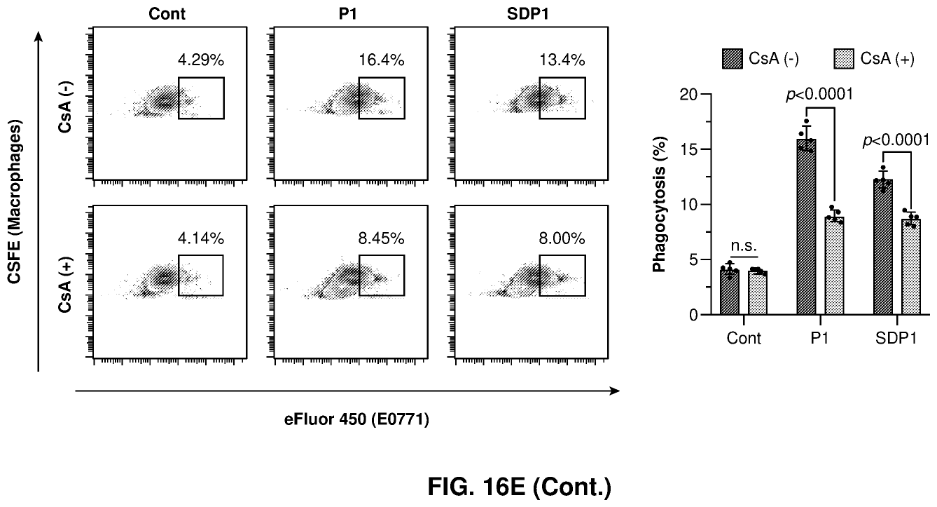

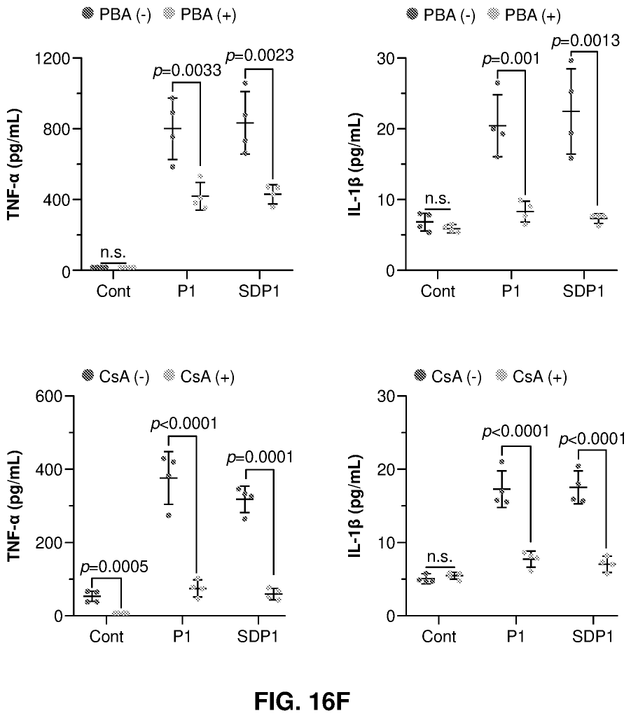

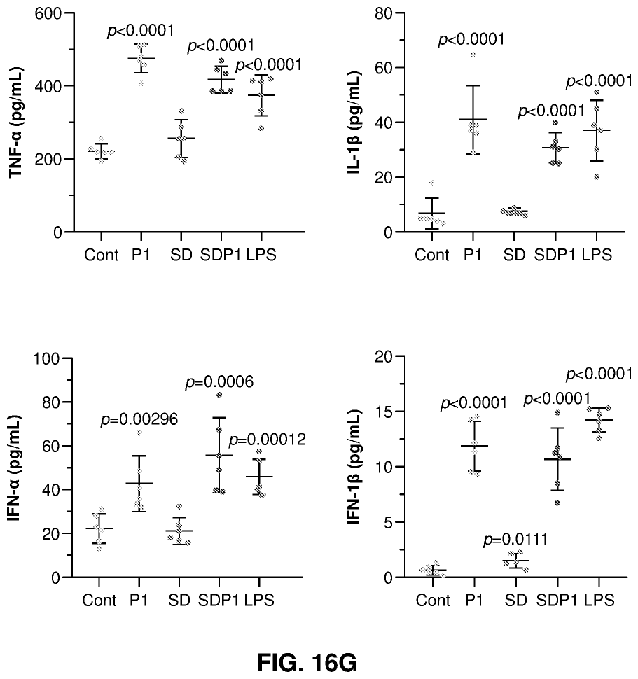

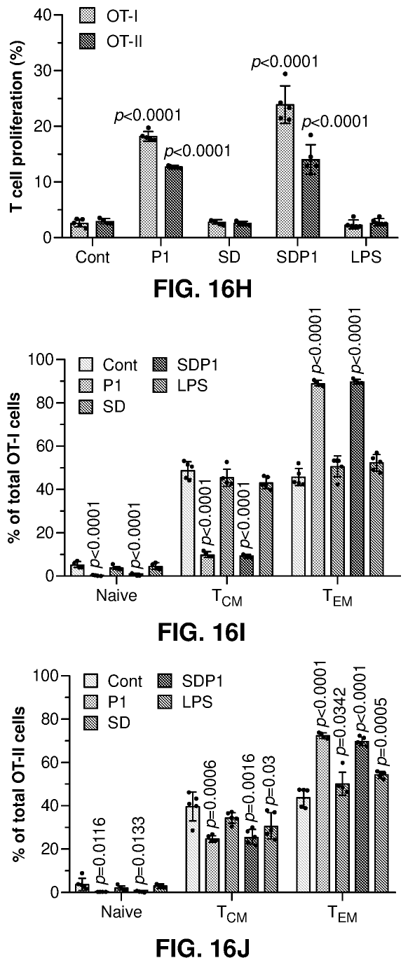

As shown in FIG. 16A and FIG. 16B, P1 consistently increased tumor cell phagocytosis by macrophages as compared to the other treatment groups.

To determine whether cationic polypeptide treatment could also promote antigen cross presentation by APCs following tumor cell phagocytosis, M2-polarized macrophages were co-cultured with E0771 cells expressing the model antigen cytoplasmic ovalbumin (cOVA).

As shown in FIG. 16C and FIG. 16D, P1 and SDP1 treatment resulted in increased cross-presentation of OVA peptide by the major histocompatibility protein I complex. However, inhibition of ER stress or mtDNA release abrogated P1 and SDP1-mediated phagocytosis of E0771 cancer cells, thus verifying that P1-mediated activation of phagocytosis was dependent on induction of ER stress and mtDNA release (FIG. 16E). Additionally, P1 and SDP1 treatment of M2-polarized macrophages also promoted the production of pro-inflammatory cytokines including tumor necrosis factor (TNF)-α, interleukin (IL)-1β, Interferon (IFN)-α and IFN-β (FIG. 16G), many of which are essential for the efficient priming of T cells. P1-induced upregulation of the pro-inflammatory cytokines was attributed to both ER stress and mtDNA release as inhibition of these processes negated the observed effects (FIG. 16F).

To determine whether polypeptide-activated macrophages can more efficiently prime antigen specific T cells, macrophages that had previously been co-cultured with E0771-cOVA cells were subsequently incubated with CD4+ or CD8+ T cells from OT-II and OT-I transgenic mice, respectively.

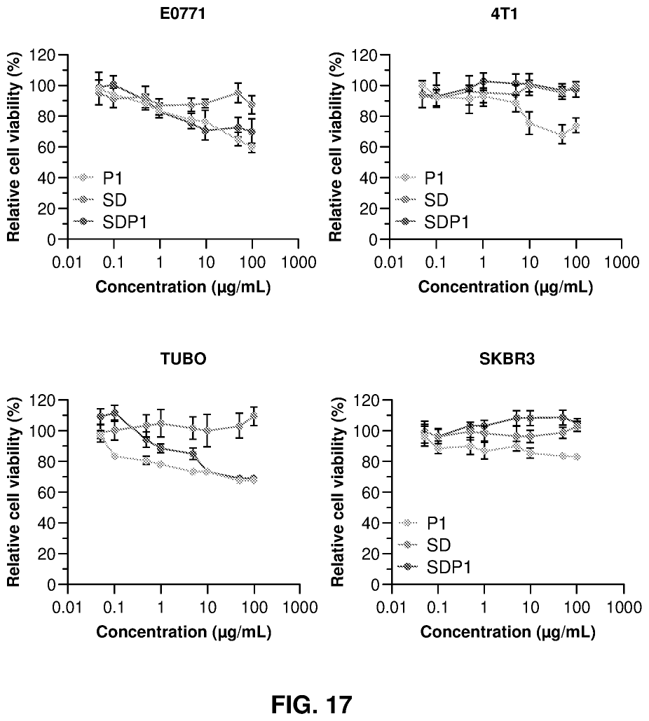

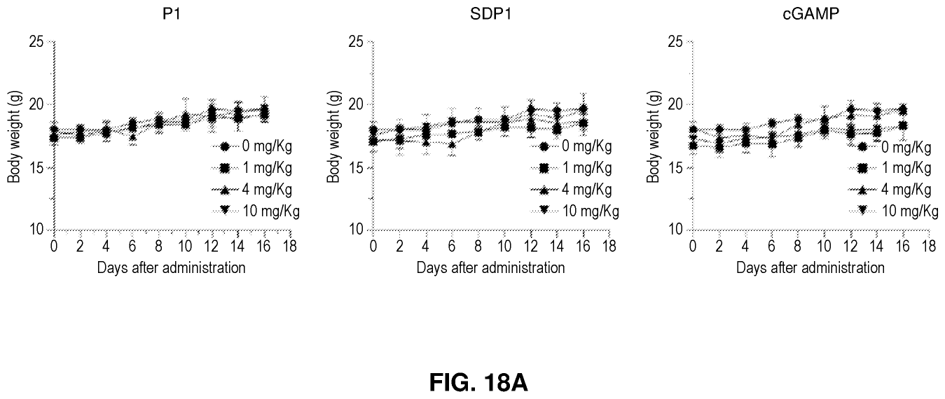

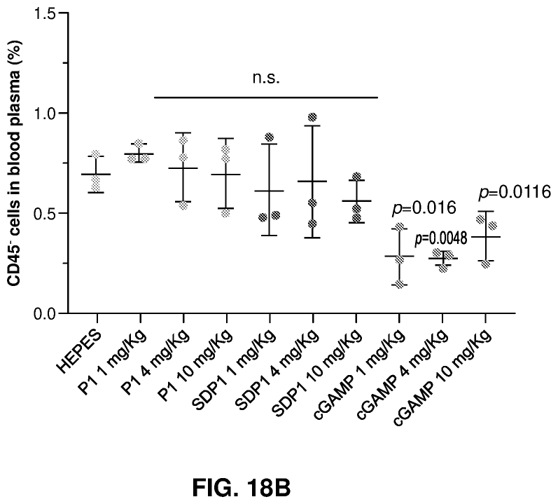

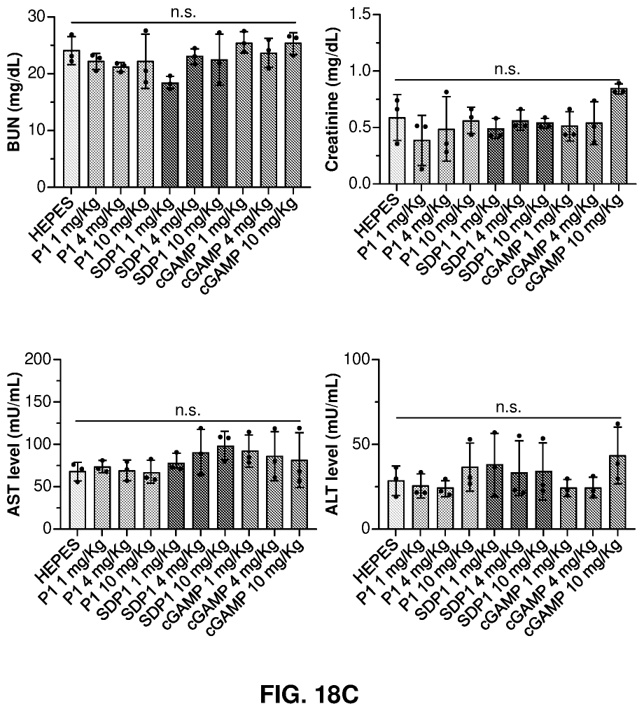

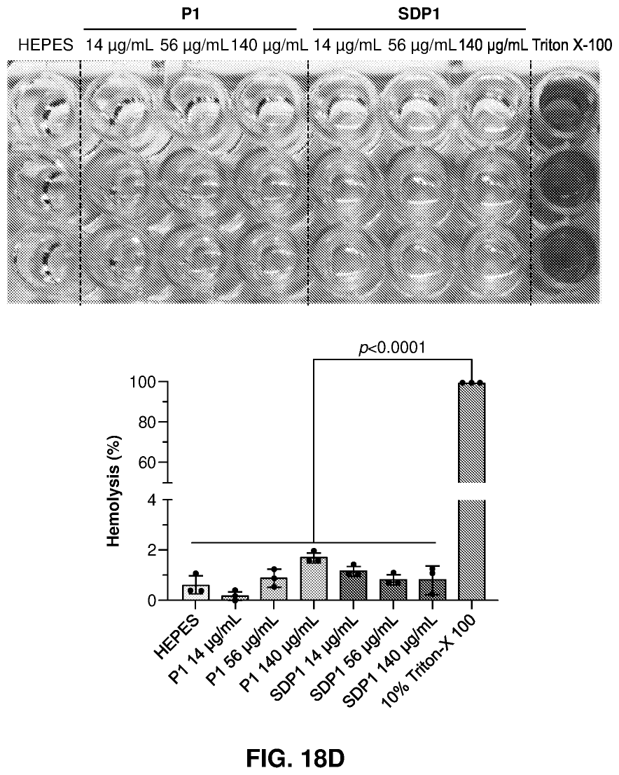

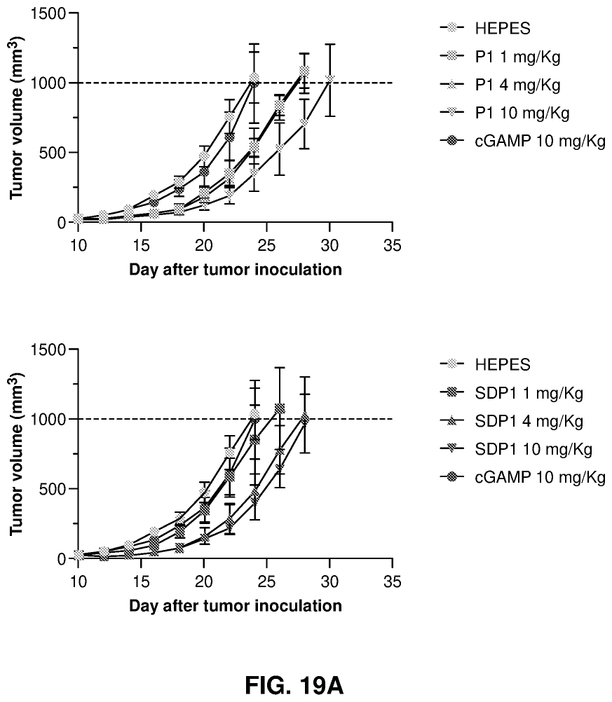

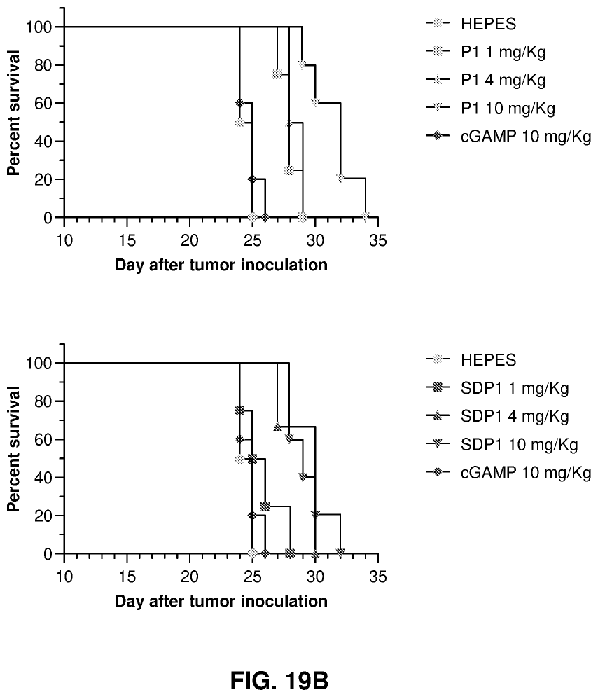

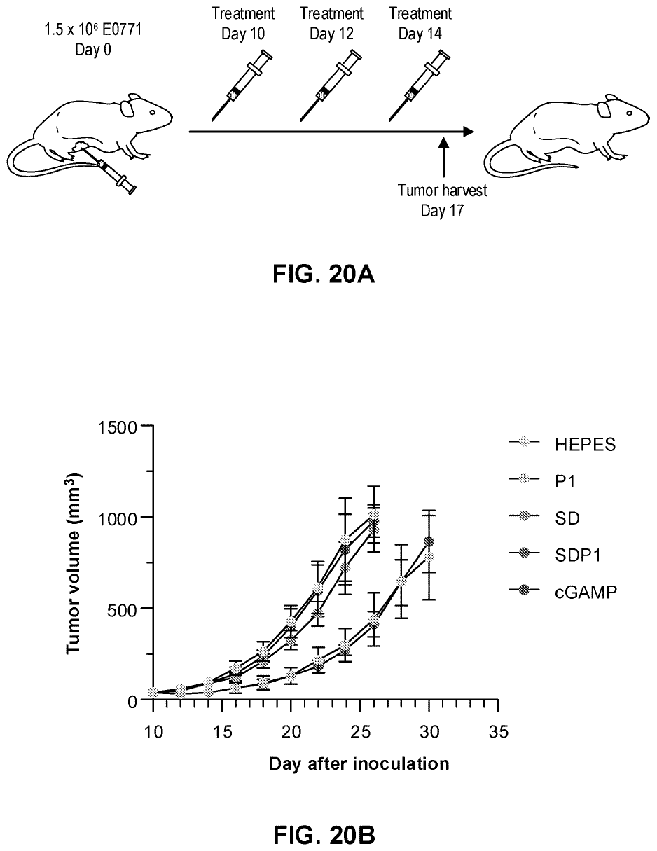

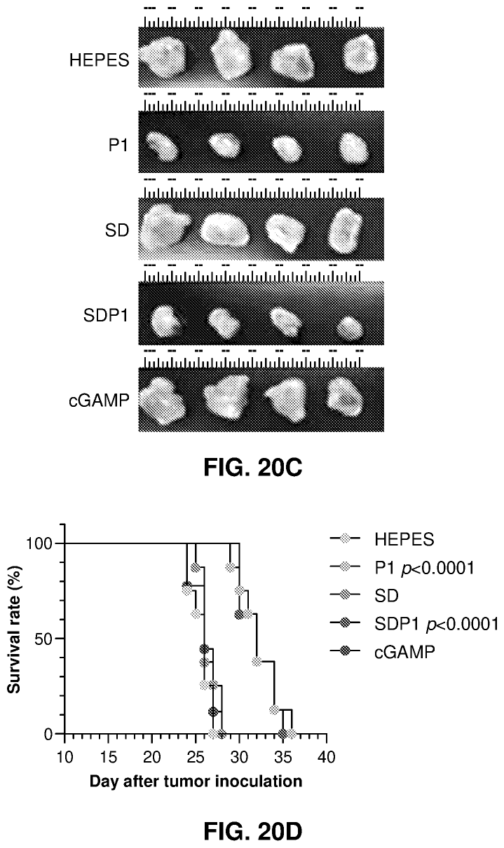

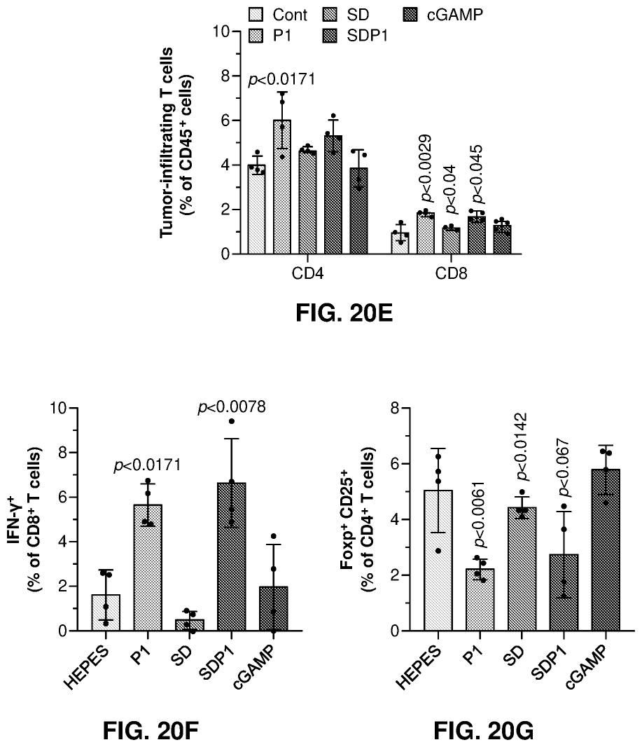

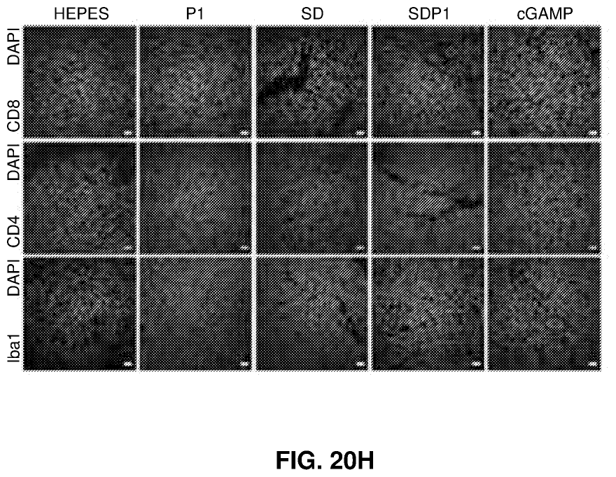

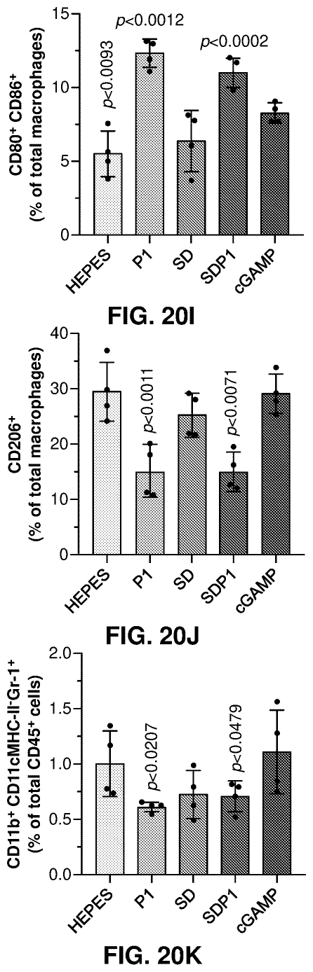

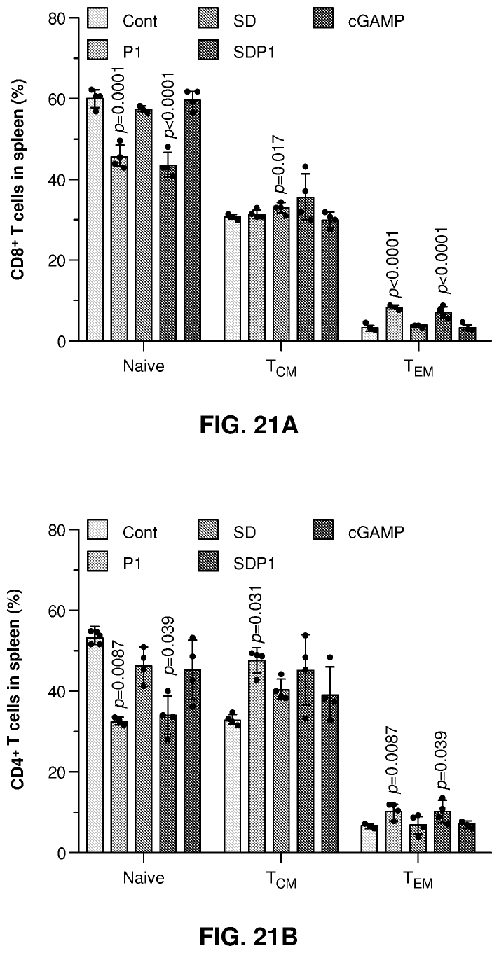

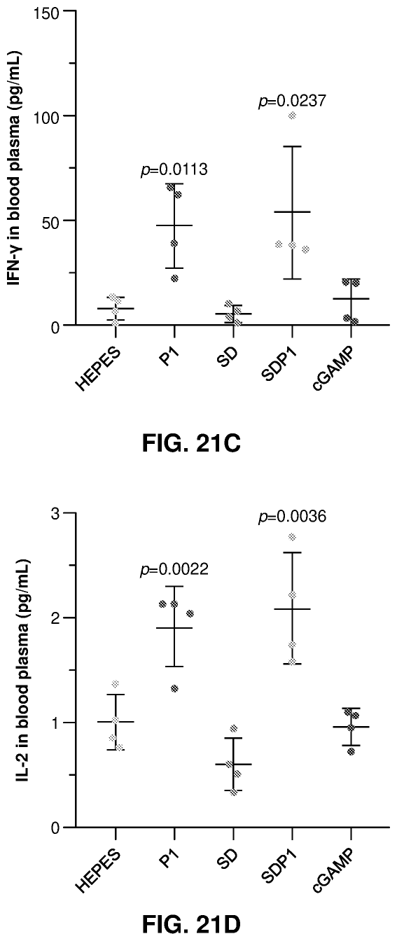

P1 or SDP1 treated groups exhibited an approximately 4-fold increase in the proliferation of OT-I and OT-II T cells (FIG. 16H and FIG. 16I). Moreover, the cationic polypeptide treatments promoted a shift toward memory phenotypes in both CD4+ and CD8+ T cells (FIG. 16J). The viability of multiple murine and human cancer cells treated with polypeptides was tested to examine whether P1-mediated immune activation results from its ability to induce immunogenic cell death of cancer cells. Neither P1 nor SDP1 induced notable cytotoxicity even at high concentrations (FIG. 17). These results together suggest that cationic polypeptide treatment promotes macrophage activation and phagocytosis of tumor cells, leading to enhance tumor antigen cross presentation and subsequent priming of memory T cells.

### Example 5: Treatment with P1 in Combination with αPD1 Produces Synergistic Antitumor Effect in EO771 Tumor-Bearing Mice

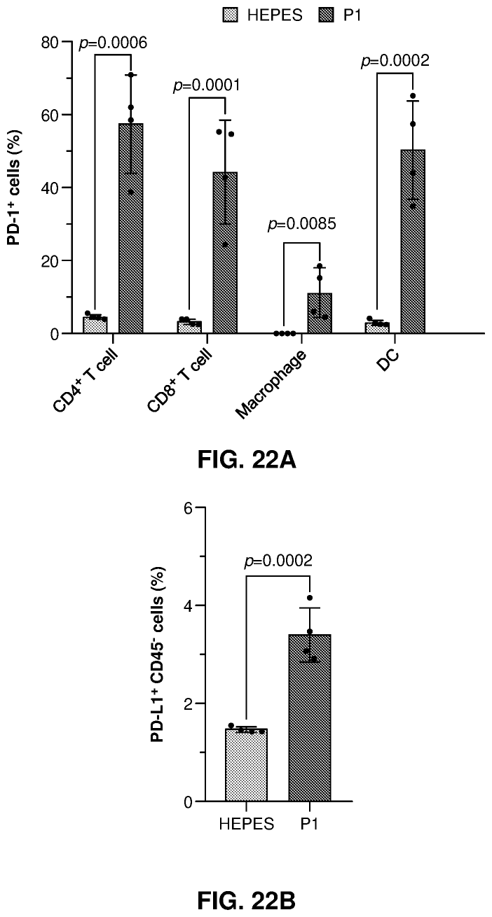

An antigen presentation assay was performed as described in Example 1, to assess whether cationic polypeptides increase the expression of programmed cell death 1 (PD1) and programmed cell death ligand 1 (PDL1) on immune cells and tumor cells, respectively. FIG. 22A and FIG. 22B show that P1-induced innate immune activation triggered upregulation of PD1 expression on tumor-infiltrating DCs, macrophages, and T cells and that of PDL1 on E0771 tumor cells.

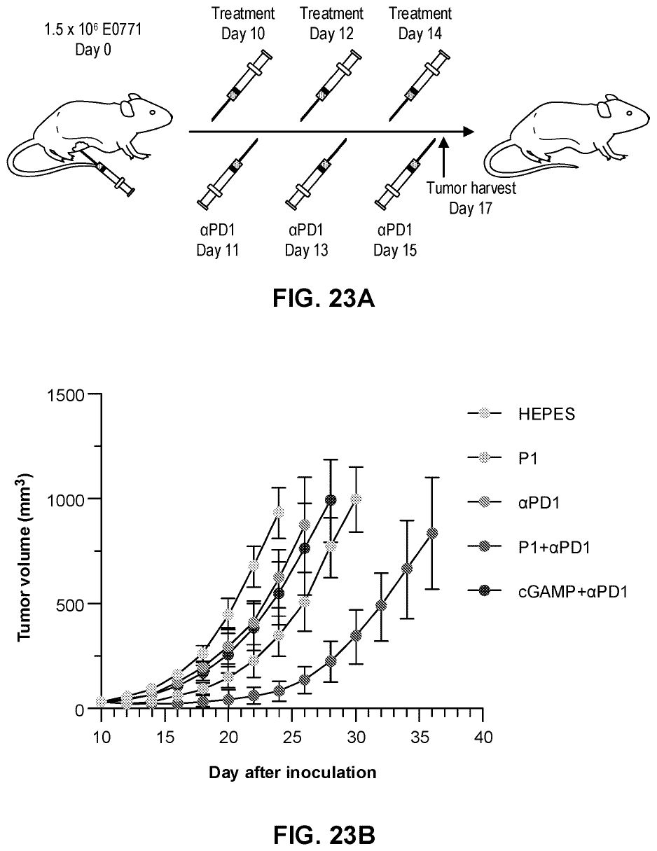

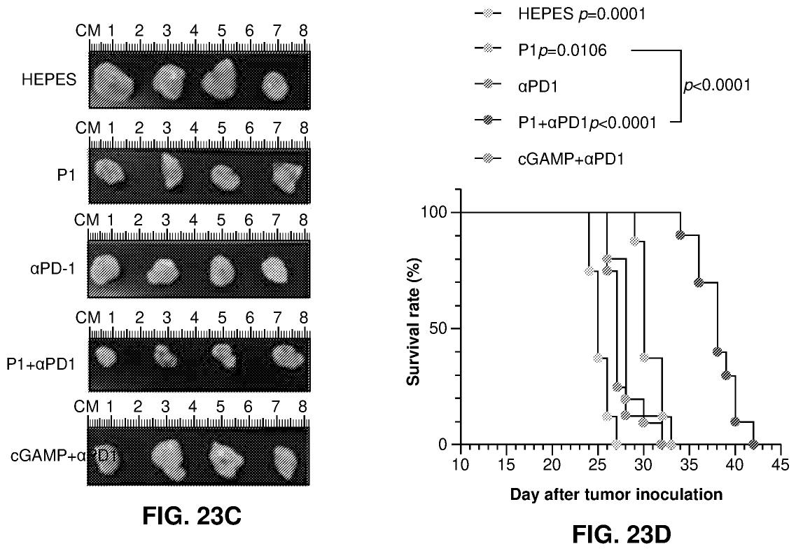

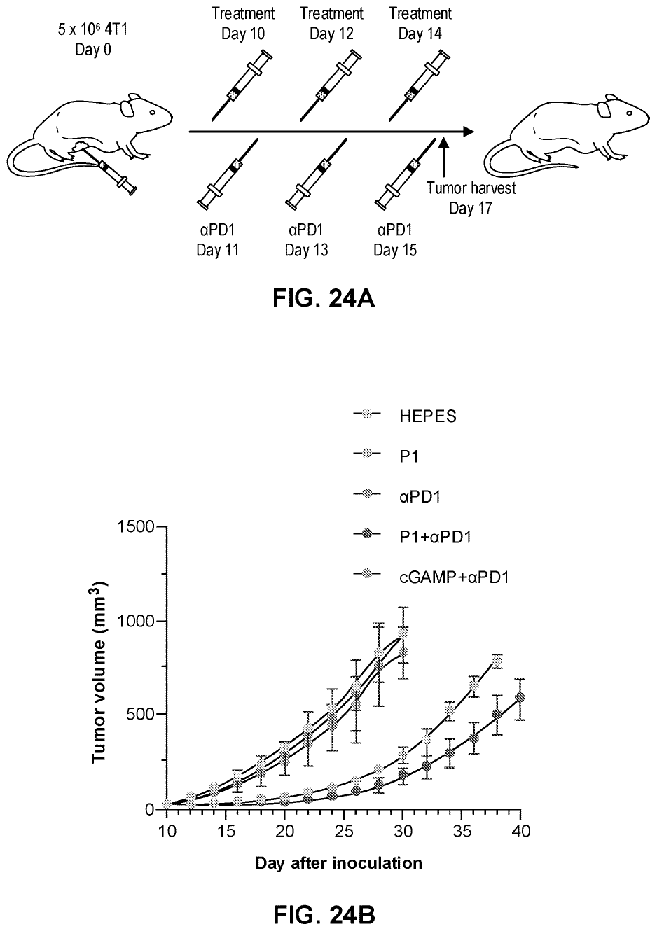

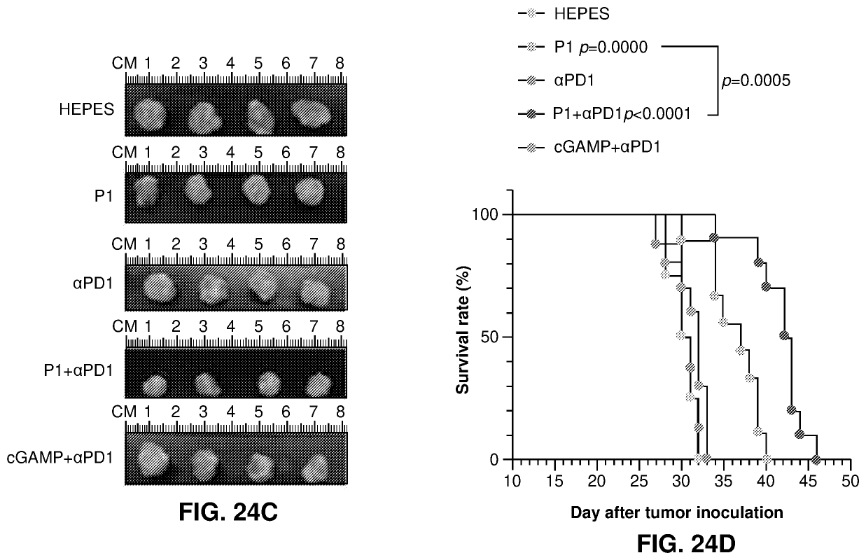

In an attempt to overcome this adaptive immune resistance, a T cell immune checkpoint blocker, αPD1, was added to the treatment. The combination of αPD1 and P1 suppressed tumor proliferation and also extended the survival of tumor-bearing mice to a greater extent than did P1 and αPD1 as monotherapy, or cGAMP+αPD1, in both E0771 and 4T1 tumor-bearing models (FIGS. 23A-D and FIGS. 24A-D).

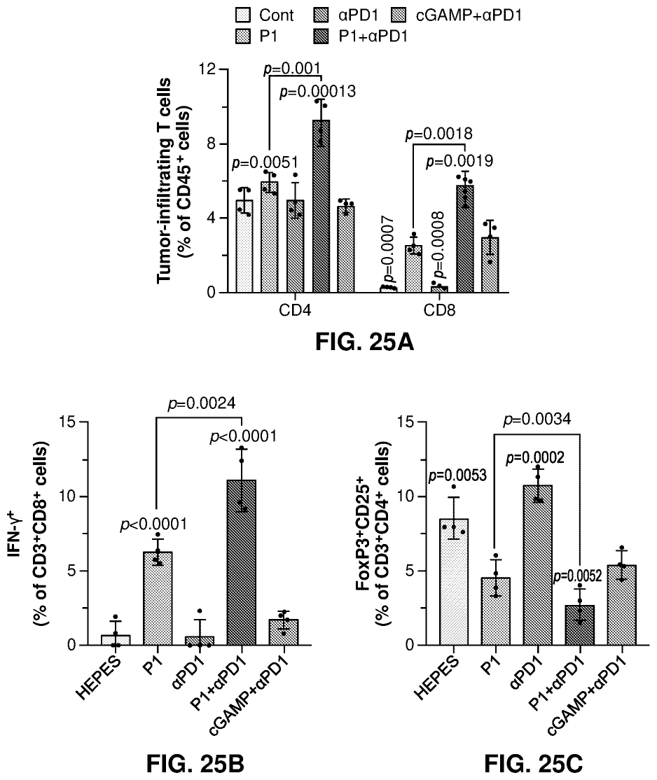

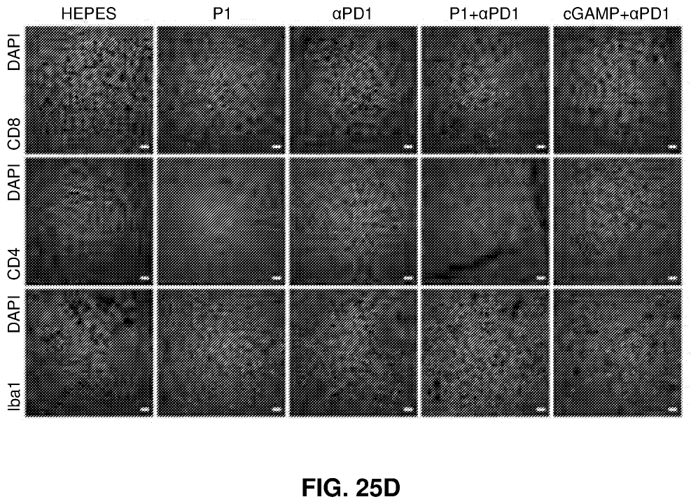

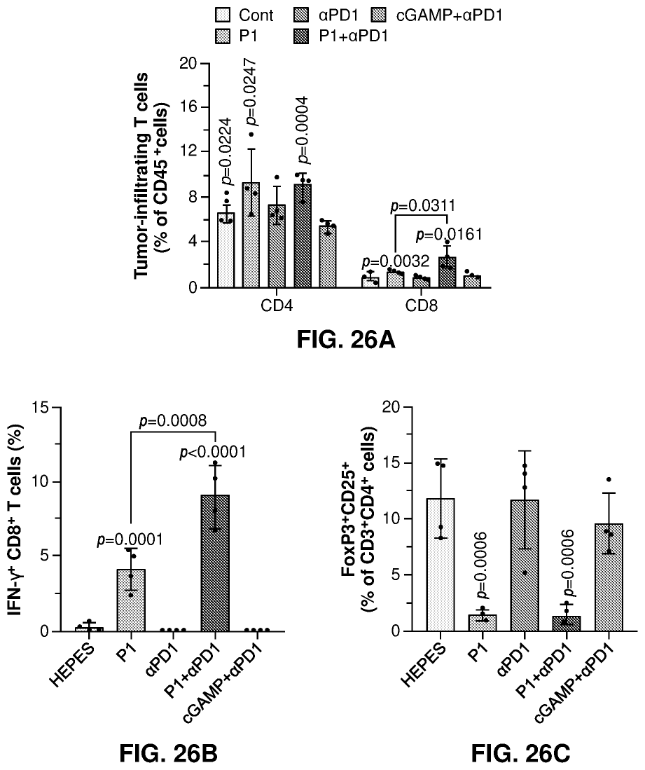

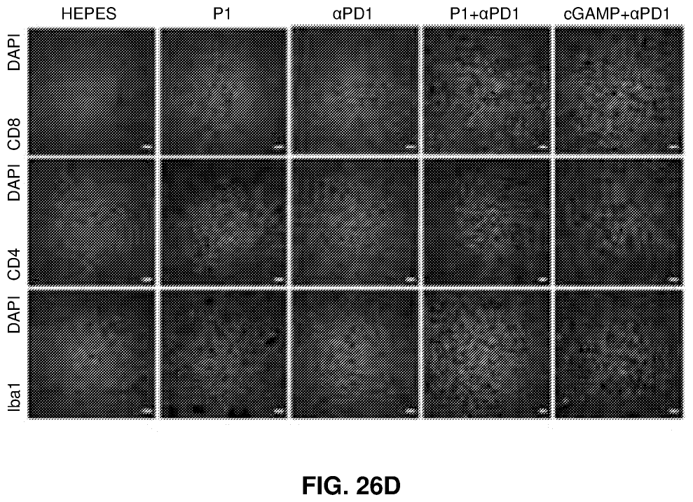

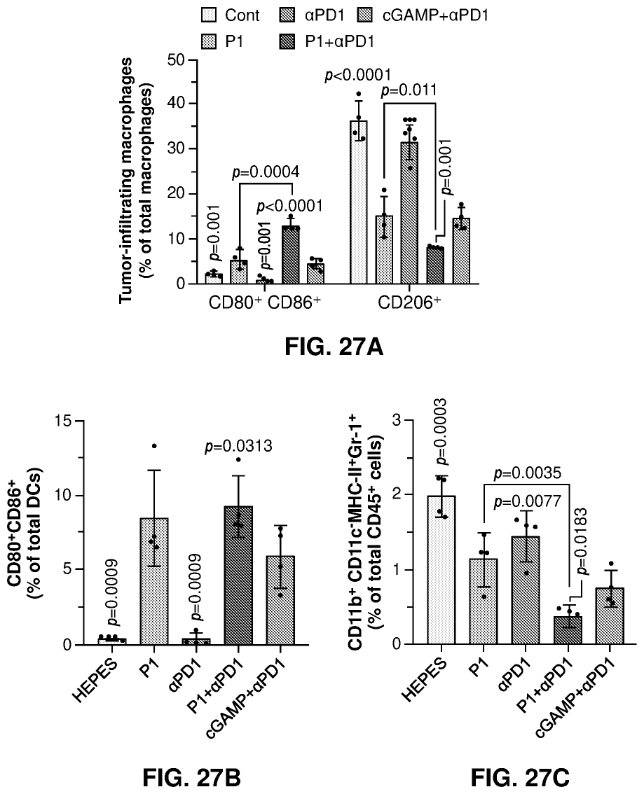

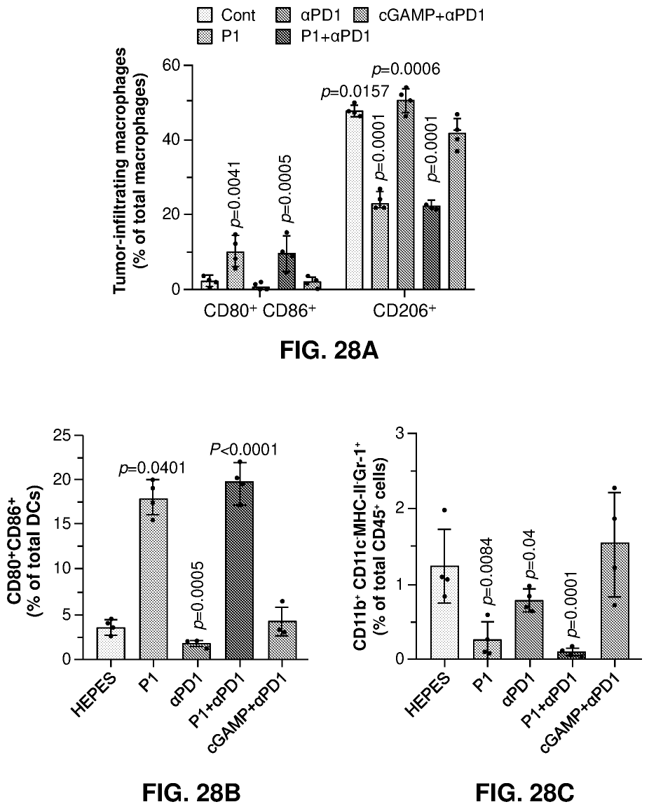

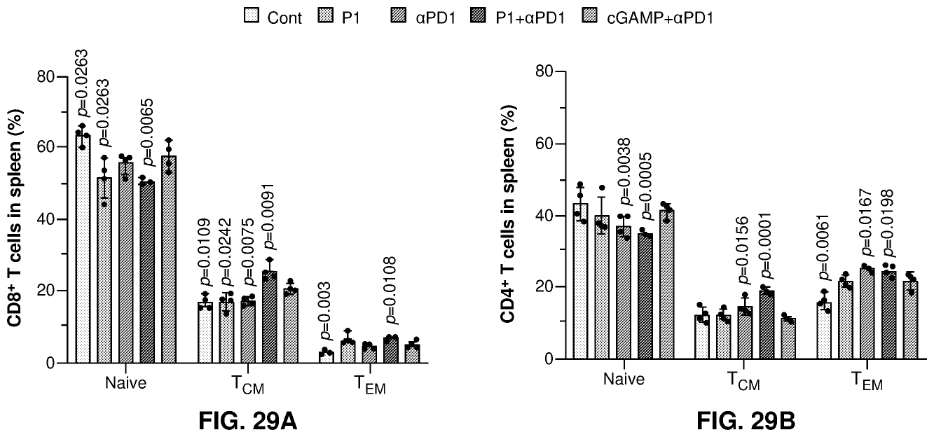

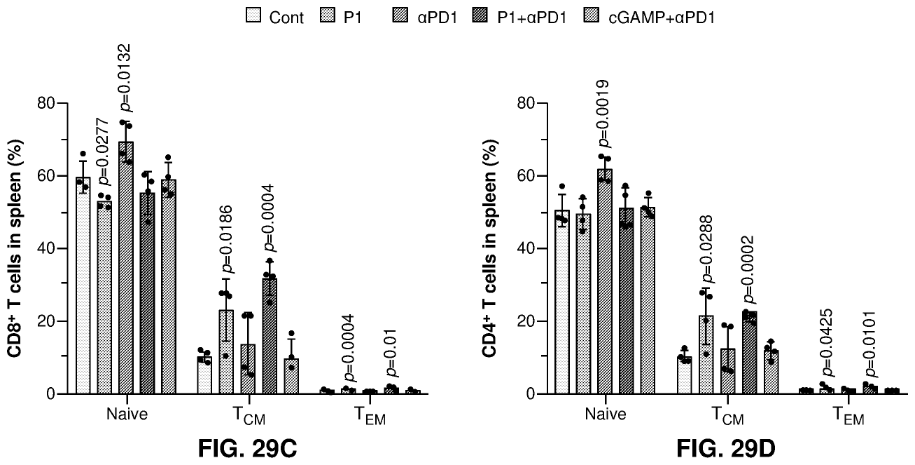

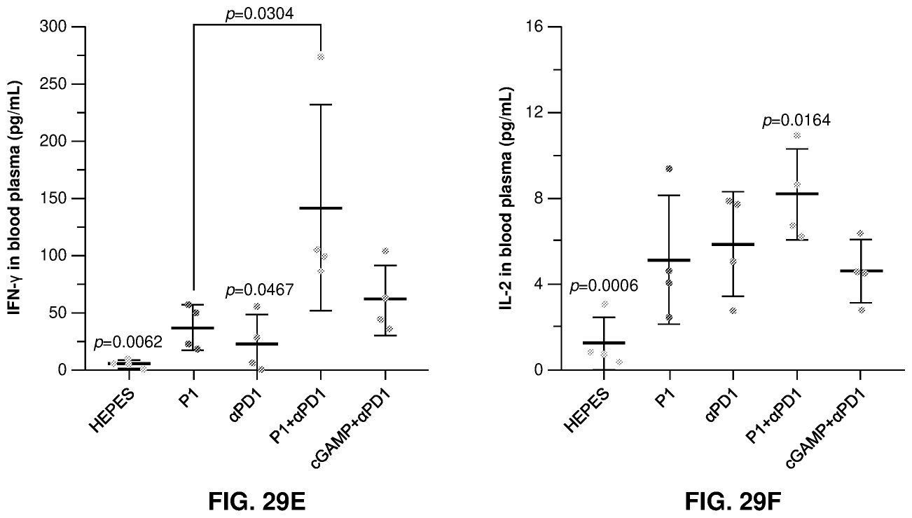

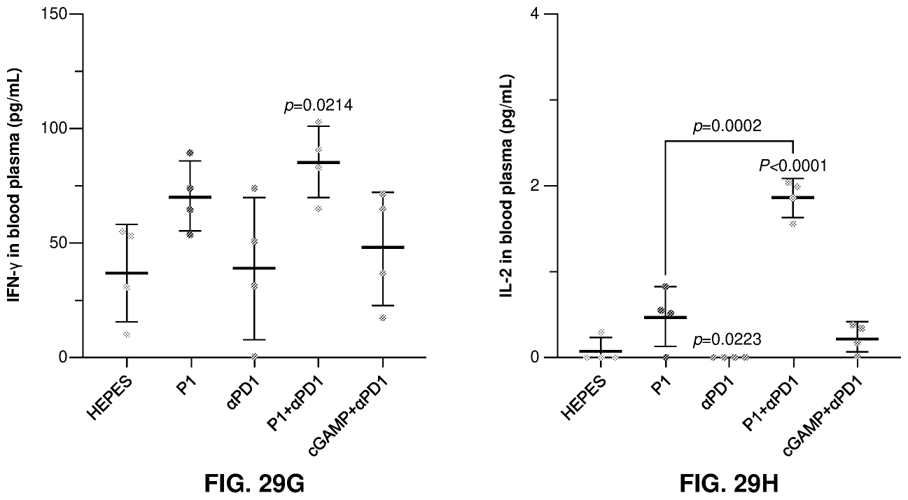

Evaluation of adaptive immune cell profiles after P1+αPD1 combination therapy demonstrated significant increases in both CD4+ and CD8+ T cells within the tumor, increases that were greater than P1, αPD1, or cGAMP+αPD1 treatments (FIGS. 25A-D and FIGS. 26A-D). Further analyses of T cell subsets confirmed that numbers of tumor-infiltrating IFN-γ+CD8+ T cells were elevated, and the population of Tregs was reduced, as compared with P1, αPD1, or cGAMP+αPD1 treatments (FIGS. 25A-C and FIGS. 26A-C). In addition, investigation of tumor-homing myeloid cell populations demonstrated that the P1+αPD1 combination treatment triggered M1 macrophage polarization, DC maturation, and Ibal upregulation, but decreased the population of tumor-associated MDSCs to a greater extent than did P1, αPD1, or cGAMP+αPD1 treatments (FIG. 25D, FIGS. 27A-C, FIG. 26D and FIGS. 28A-C). These results together suggest that the cationic polypeptide combined with blockade of a T cell immune checkpoint shifted the composition of tumor-infiltrating immune cells toward tumor-suppressive mode by activating both innate and adaptive immune responses.

### Example 6: P1-+αPD1-Induced Immune Responses Require Activation of STING and MyD88

As combined therapy elicited synergistic antitumor effects by activating both innate and adaptive immunity, it was next examined whether P1+αPD1 therapy requires stimulation of STING and MyD88 to boost antitumor immunity.

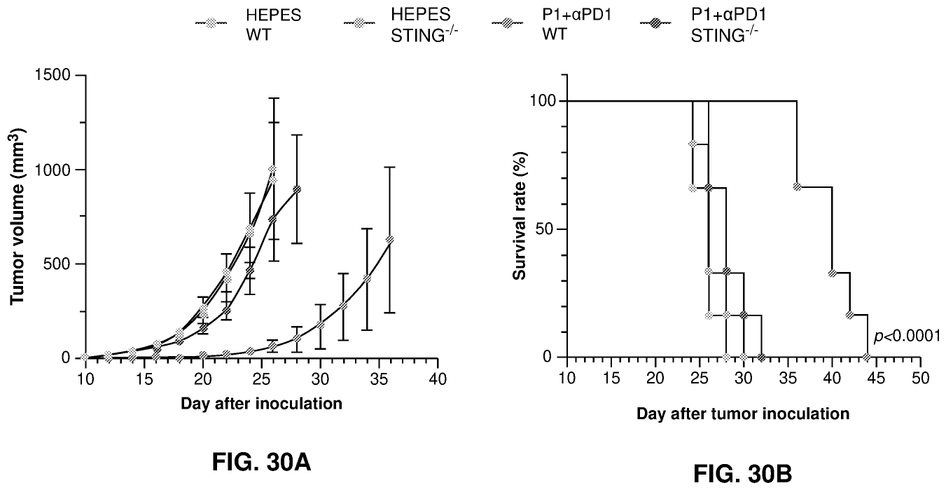

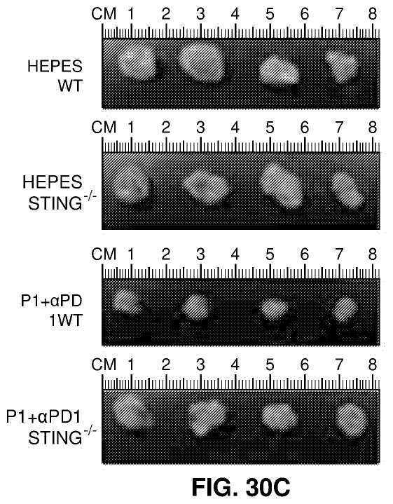

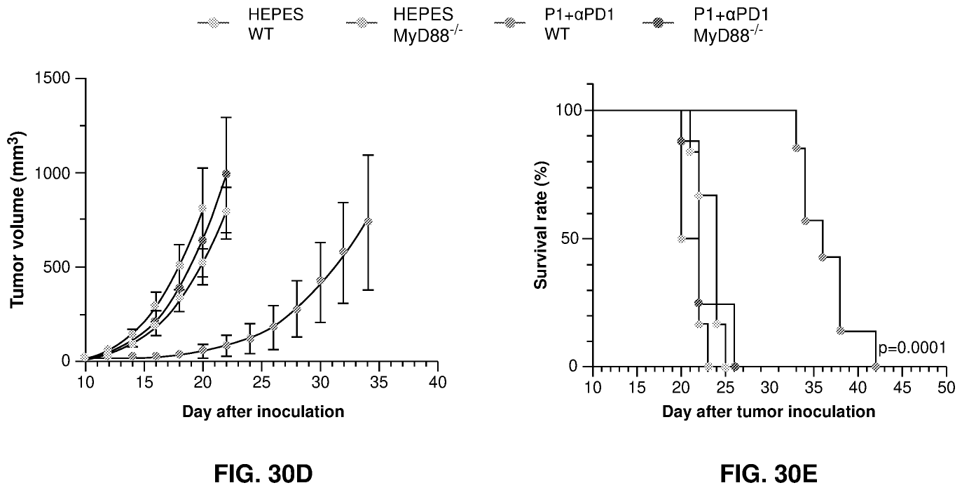

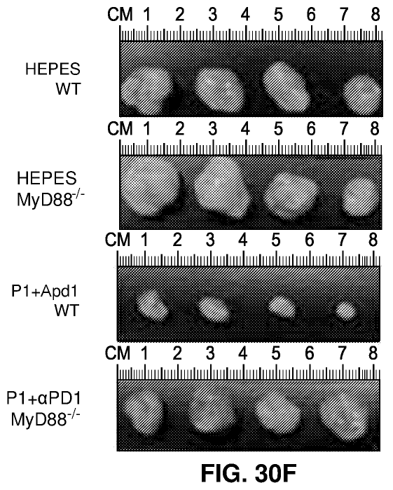

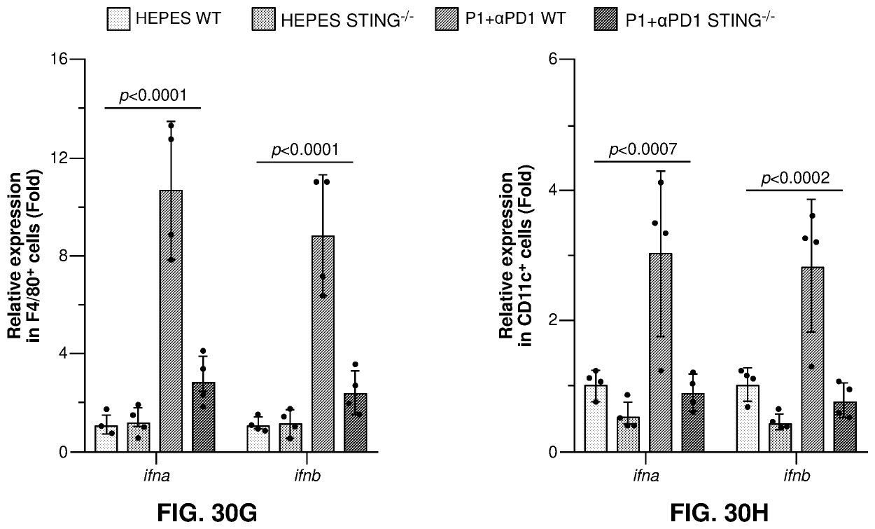

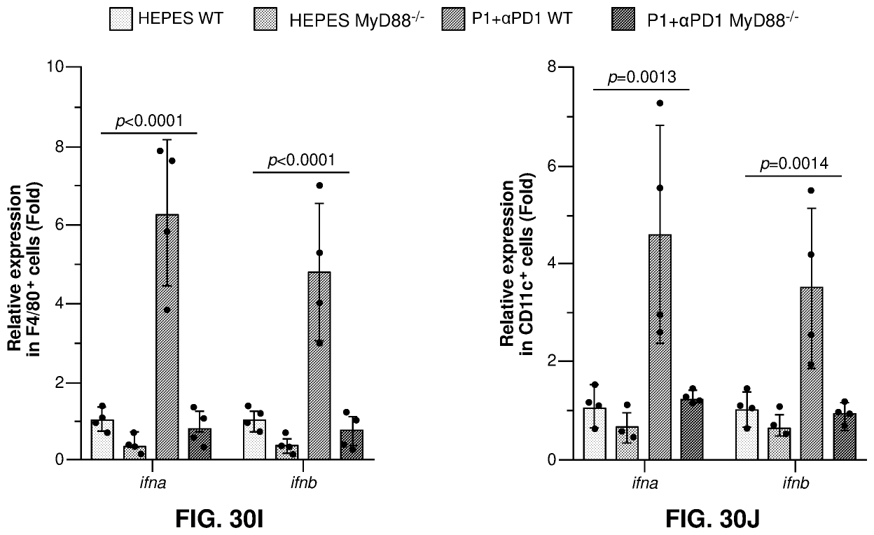

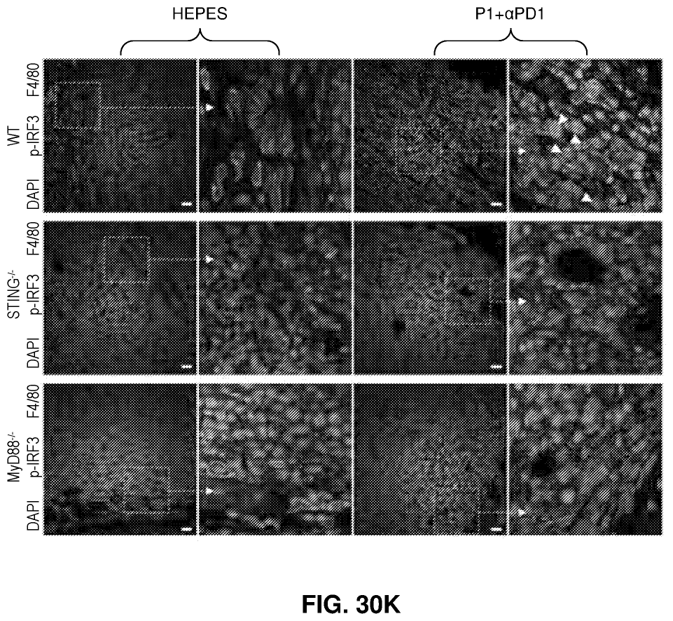

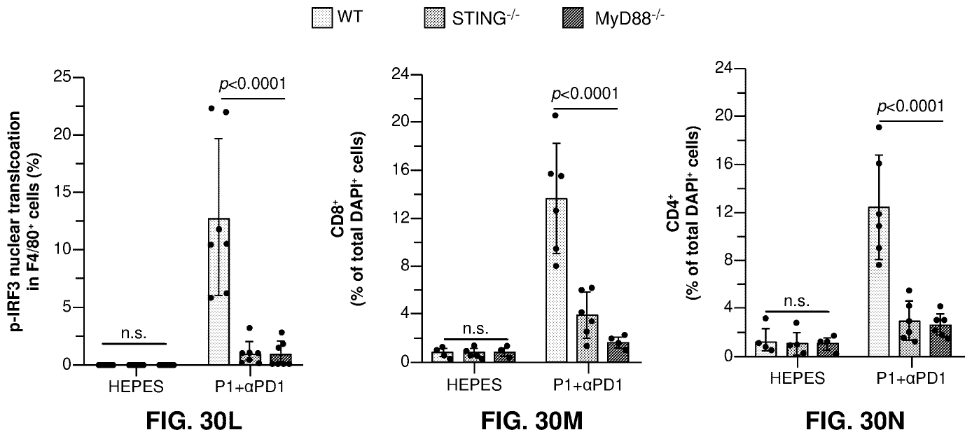

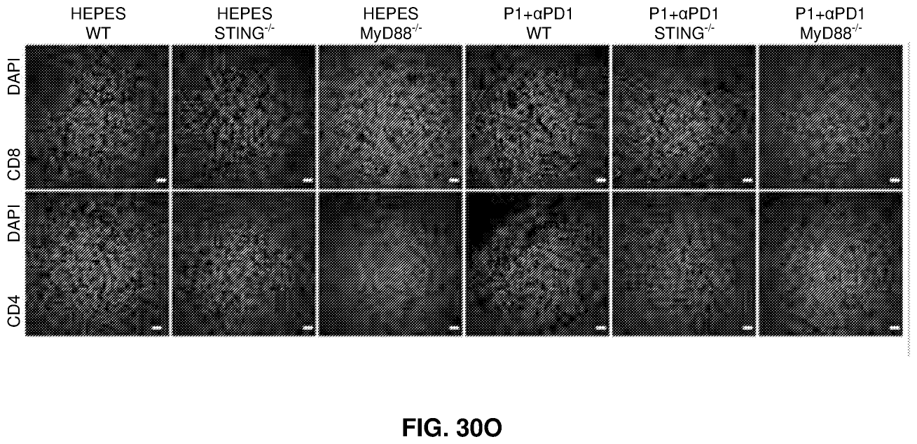

Tumor-bearing mice were established by implanting EO771 cells in the mammary fat pad of wild-type (WT), STING−/−, and MyD88−/−mice. The deficiency of STING and MyD88 not only abolished the tumor-inhibitory effects of P1+αPD1 but also reduced the survival of tumor-bearing mice (FIGS. 30A-F), thus demonstrating that activation of these innate immune sensors is required for P1+αPD1-induced antitumor responses. It was further verified that P1+αPD1 augmented type I IFNs in tumor-infiltrating APCs by using reverse transcription quantitative polymerase chain reaction (RT-qPCR) as described in Example 1. Evaluation of type I IFN gene expression in APCs revealed that the P1+αPD1 treatment significantly increased ifna and ifnb mRNAs in tumors from WT but not from STING−/− or MyD88−/− mice (FIGS. 30G-J).

Next it was examined whether P1+αPD1 treatments triggered nuclear translocation of phosphorylated IRF3 (p-IRF3) through MyD88 and STING signaling to generate type I IFN responses. Immunofluorescence analysis, as described in Example 1, of the p-IRF3 in macrophages showed that the combination treatment triggered nuclear translocation of p-IRF3 in macrophages in WT but not in STING−/− or MyD88−/− mice (FIGS. 30K-M), confirming that propagation of both MyD88 and STING signaling pathways is required for P1+αPD1-mediated generation of type I IFN responses.

Considering that type I IFNs have key roles in recruiting cytotoxic T cells within tumors, tumor-infiltrating T cells were visualized by immunofluorescence staining for CD8 and CD4. The knockout of STING or MyD88 abrogated the recruitment of CD8+ and CD4+ T cells within tumors in the P1+αPD1-treated mice (FIG. 30N and FIG. 30O), implying that activation of STING and MyD88 signaling is important for P1+αPD1-mediated activation of adaptive immunity.

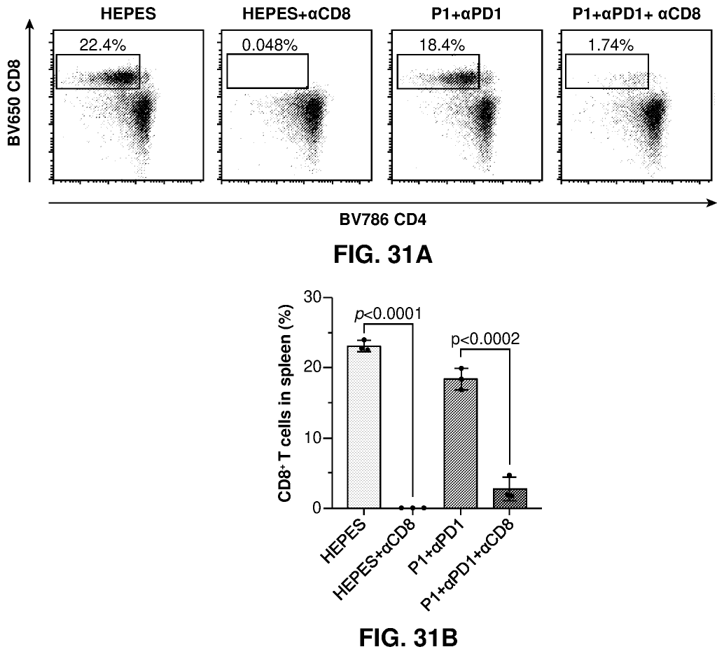

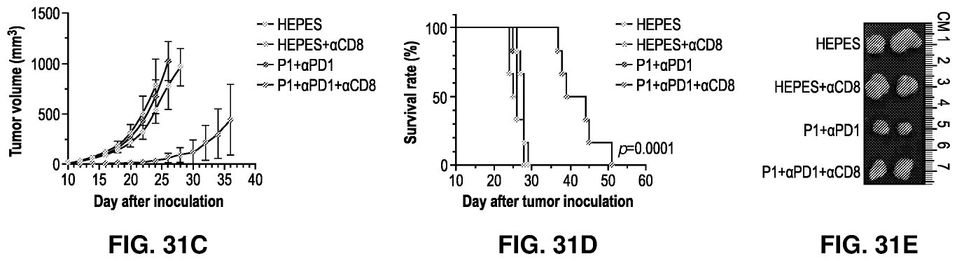

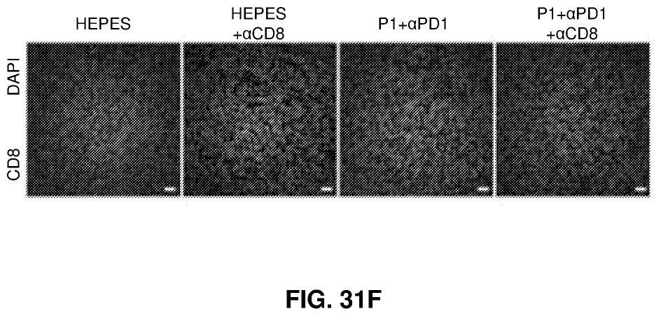

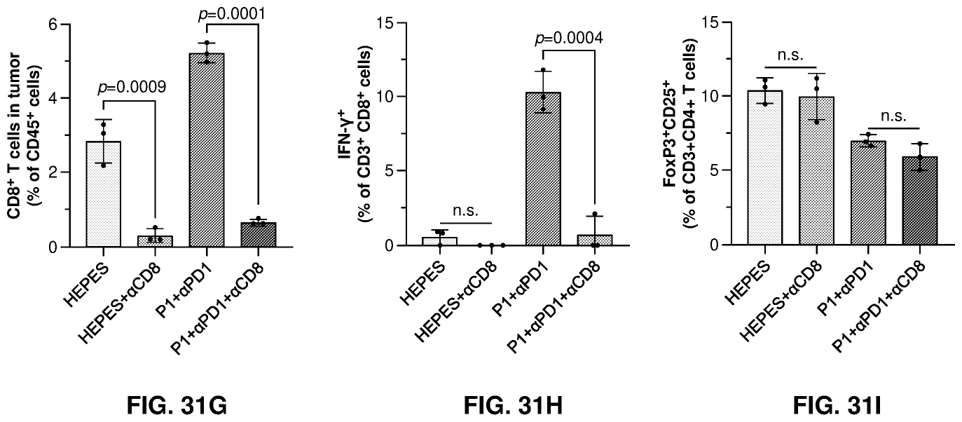

To examine whether antitumor effect of P1+αPD1 combination treatment depends mainly on CD8+ T cell-mediated adaptive immunity, CD8+ T cells were depleted in EO771 tumor-bearing mice (FIGS. 31A and 31B). C57/BL6 mice were injected intraperitoneally with anti-CD8a mAbs (300 g, BioXcell, USA) every 4 days beginning 3 days before the various treatments. CD8+ T cell depletion was confirmed by flow cytometry of CD8+ T cells isolated from the spleens of the mice.

CD8 T cell depletion completely abrogated the antitumor immunity conferred by P1+αPD1 and also shortened the survival of tumour-bearing mice (FIGS. 31C-31E). The tumor-infiltrating T-cell profile in the P1+αPD1 group revealed that CD8 depletion not only blocked recruitment of CD8+ T cells but also decreased the production of IFN-γ while maintaining Treg proportions (FIG. 31F and FIGS. 31G-311). These experimental outcomes suggested that the tumoricidal effect of P1+αPD1 combination treatment was largely mediated by CD8+ T cells.

### Example 7: Treatment with P1+αPD1 Suppresses the Metastasis of Aggressive Breast Tumors

Residual tumors or circulating tumor cells can cause recurrence and metastasis even after surgical resection of the primary tumor. BR4, a 4T1-braintropic, aggressive metastatic breast cancer cell line, was established by repeated selection and was used to test whether P1 combined with αPD1 would be effective in treating aggressive metastatic tumors.

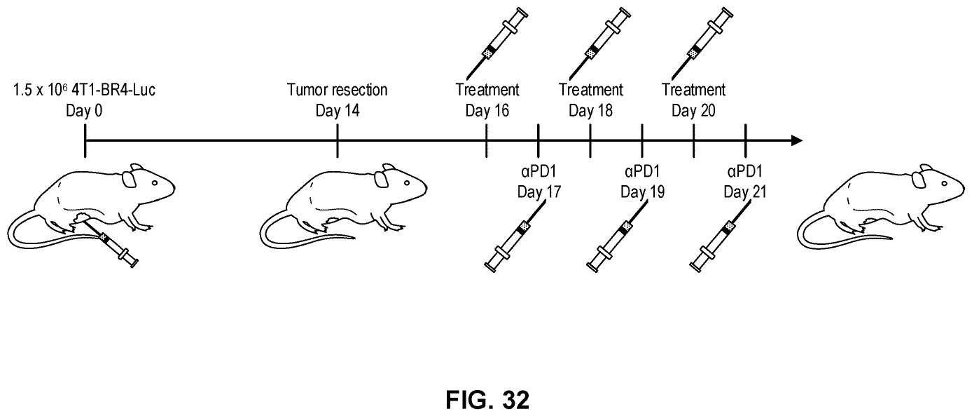

The spontaneous metastatic breast tumor model was established by injecting 4T1-BR4-mcherry Luc cells (1.5×10⁶ cells/50 μL) into the mammary pad, after which tumors were resected 14 days later (FIG. 32). Treatments were implemented on day 16, 18, and 20 for P1 and cGAMP (20 mg/kg, intravenous injection) or day 17, 19, and 21 for αPD1 (200 μg, intraperitoneal injection). Spontaneous 4T1 metastases were visualized with an IVIS spectrum imaging system (IVIS Lumina XR, Perkin Elmer).

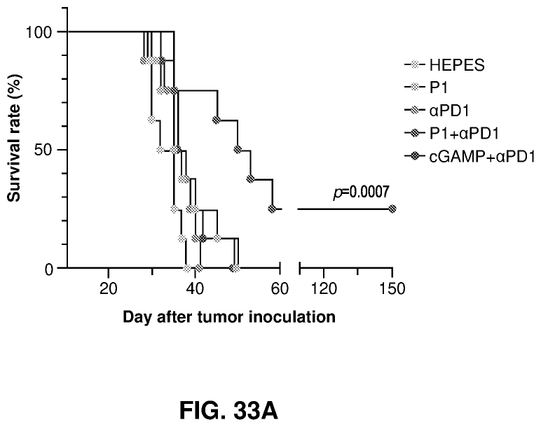

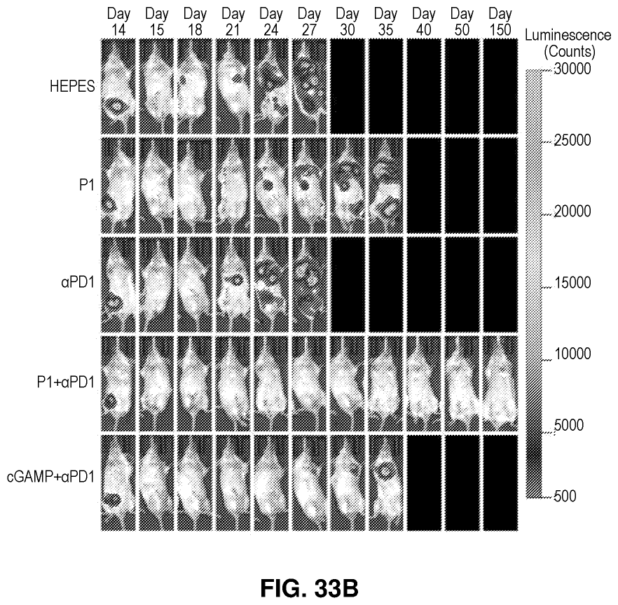

Using bioluminescence imaging to track the 4T1-BR4-Luc in vivo, it was seen that P1+αPD1 treatments suppressed tumor metastasis for a longer period (Day 150) than did the other treatment conditions (FIG. 33A and FIG. 33B). Moreover, two of the eight mice in the P1+αPD1 treatment group were tumor-free and survived up to day 150 (FIG. 33A and FIG. 33B). Although P1 or αPD1 given separately did not have evident effects, co-treatment with P1+αPD1 led to a drastic improved therapeutic effect in breast tumor model of aggressive metastasis (FIG. 33A and FIG. 33B). Moreover, the ability of P1+αPD1 to suppress aggressive tumor metastases surpassed that of cGAMP+αPD1, suggesting that the P1+αPD1 combination was much more effective in treating these aggressive metastatic tumors (FIG. 33A and FIG. 33B).

### Example 8: Polypeptide Design Optimization by Varying Hydrophobicity, Electrostatic Charge, and Side Chain Length

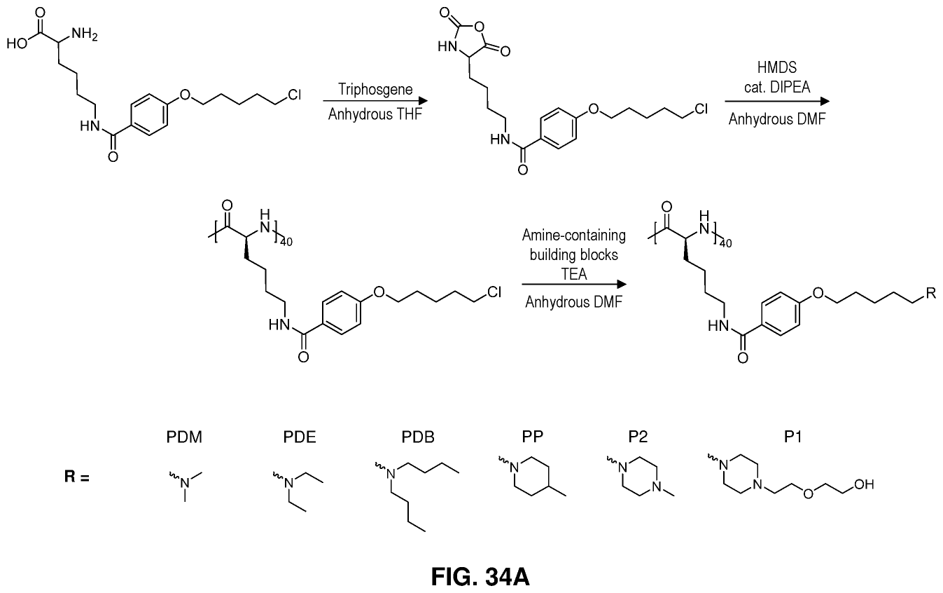

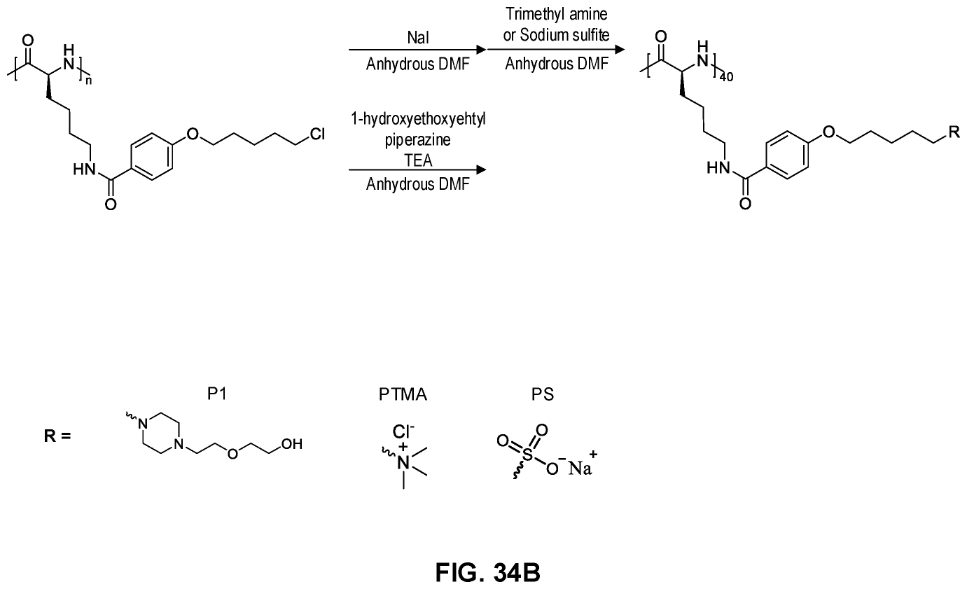

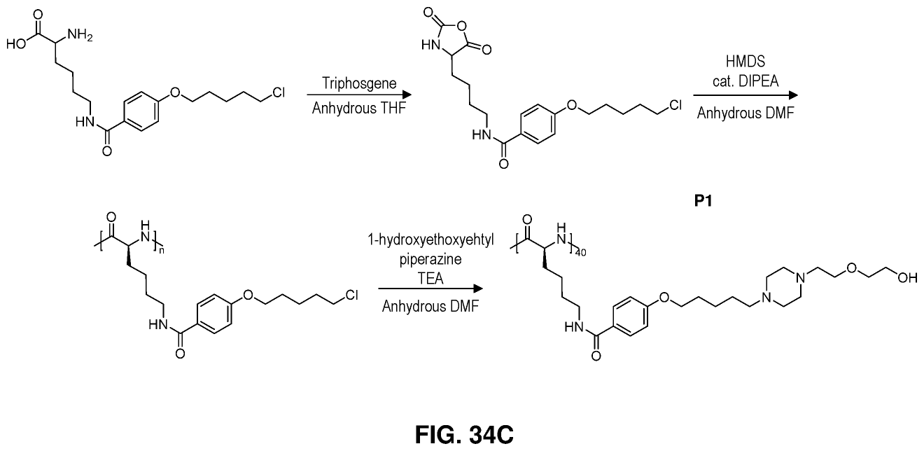

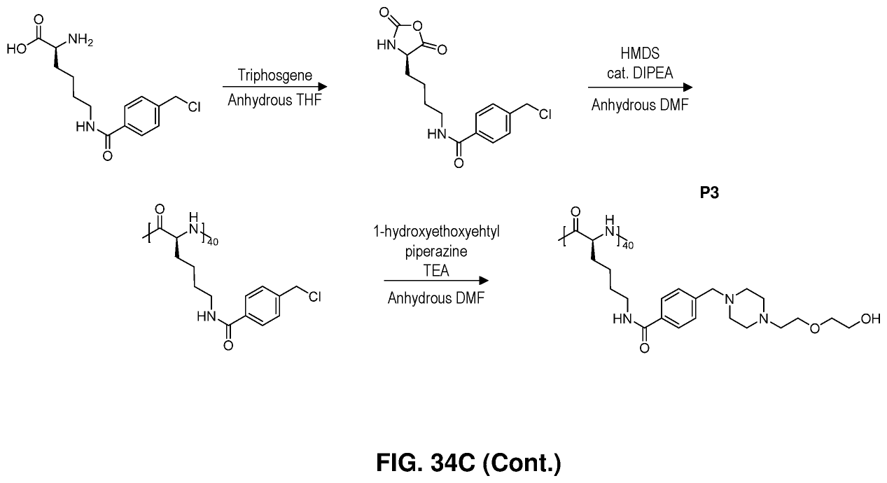

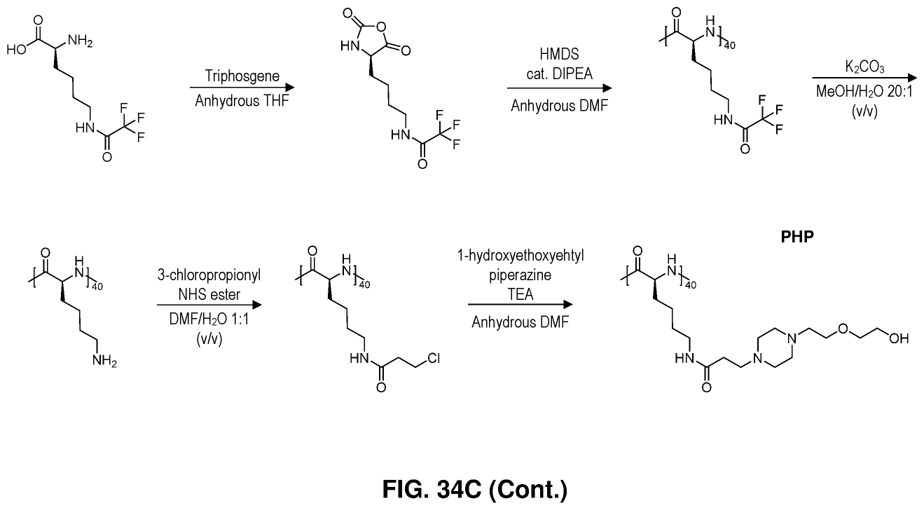

A three step optimization process was conducted to find the optimal polypeptide that can generate pro-inflammatory responses in innate immune cells. Step 1 of the three step optimization process was modulation of hydrophobicity. Various analogues (PDM, PDE, PDB, PP, P2 and P1) were prepared as shown in FIG. 34A. The analogues' immunogenicity was determined via the mtDNA release assay and phagocytosis assay as described in Example 1.

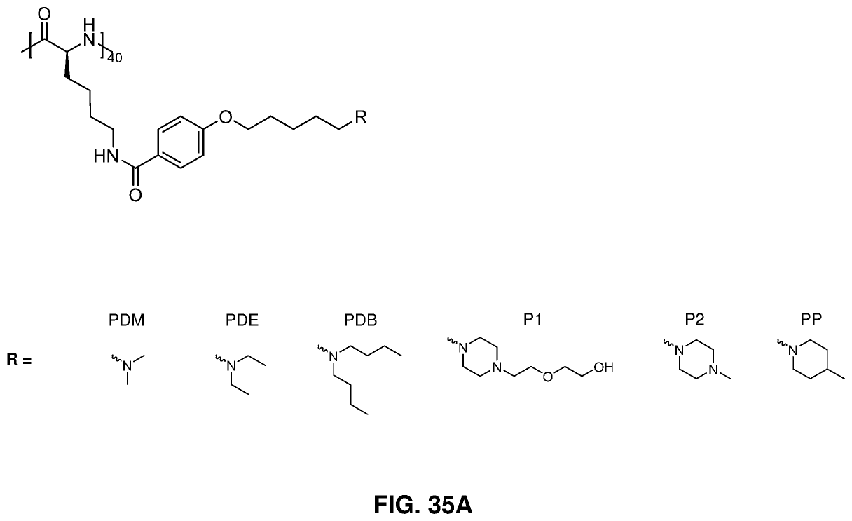

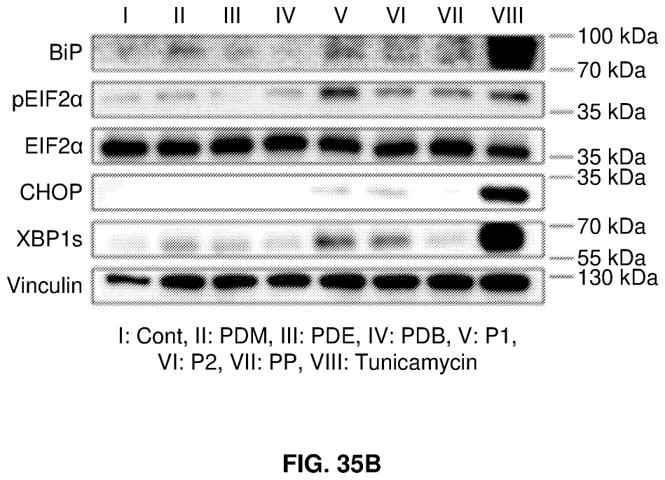

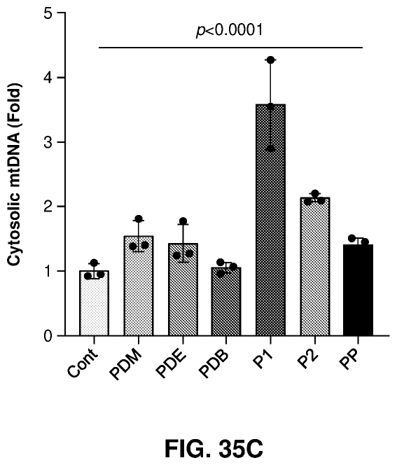

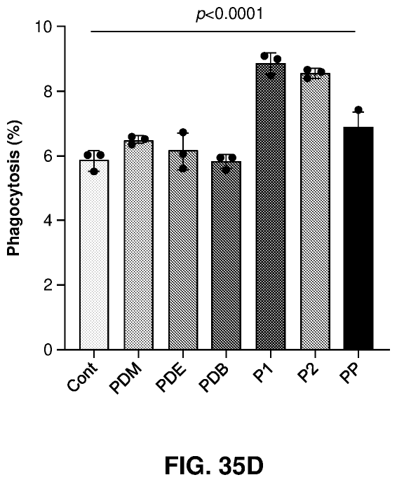

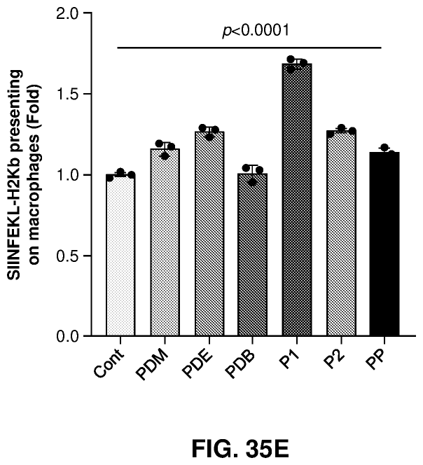

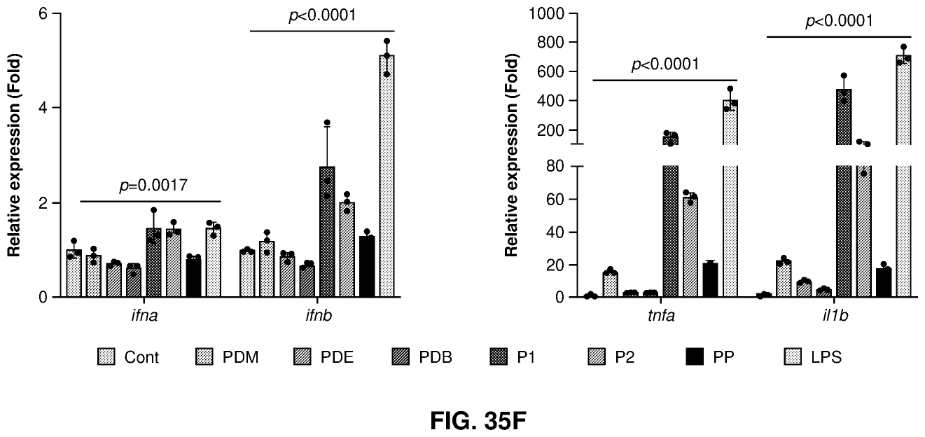

As can be seen in FIGS. 35B-35F, cationic polypeptides including hydrophilic analogues and cyclic structures induced ER stress and mtDNA release in bone marrow-derived macrophages (BMDMs). The results showed that cationic polypeptides tethered with a hydrophilic building block and cyclic structure increased phagocytosis of EO771 breast cancer cells (FIG. 35D) and cross-presentation of the model antigen SIINFEKL-H2Kb (FIG. 35E). The varying hydrophobicity of the analogues also affected gene expression of pro-inflammatory cytokines (FIG. 35F). The results of step 1 suggest a polypeptide tethered with a hydrophilic building block and cyclic structure induces greater pro-inflammatory responses in innate immune cells.

Step 2 of the optimization process was modulation of electrostatic charge. Various analogues (P1, PTMA, and PS) were prepared as shown in FIG. 34B, and their immunogenicity was determined via the mtDNA release assay and phagocytosis assay as described in Example 1.

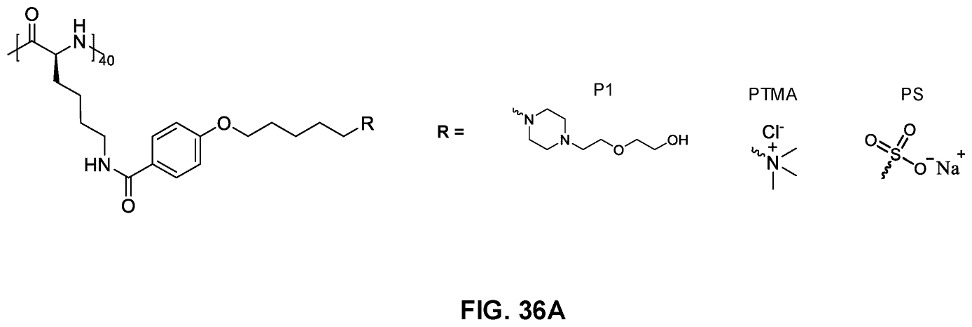

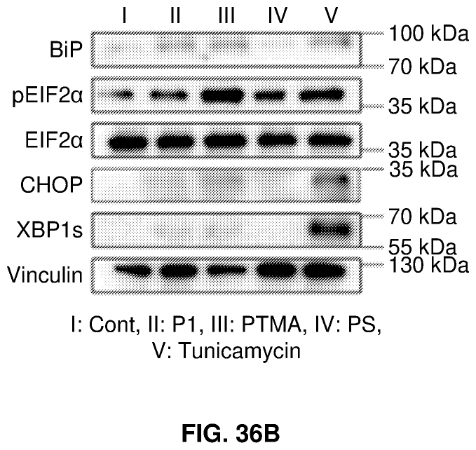

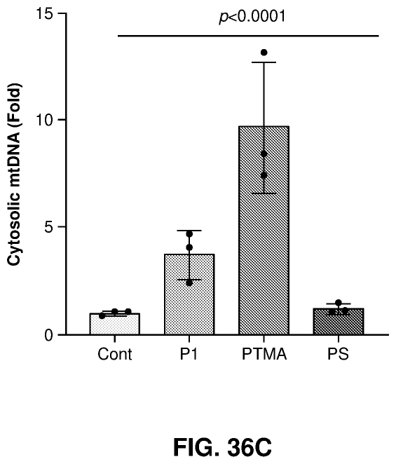

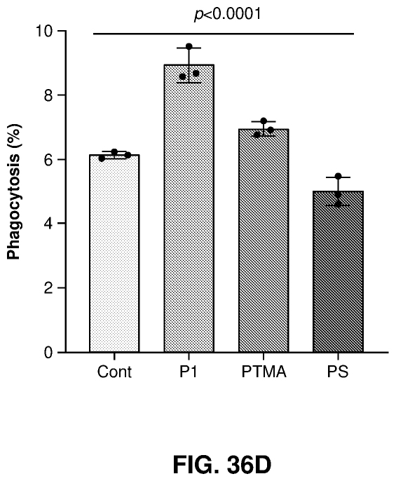

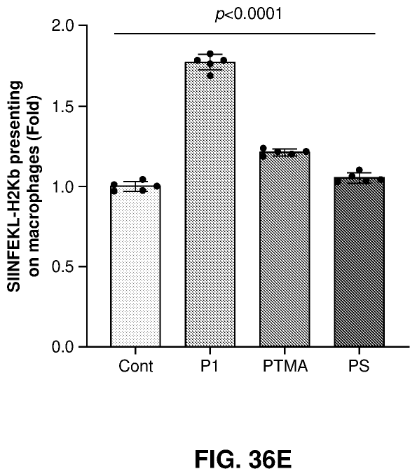

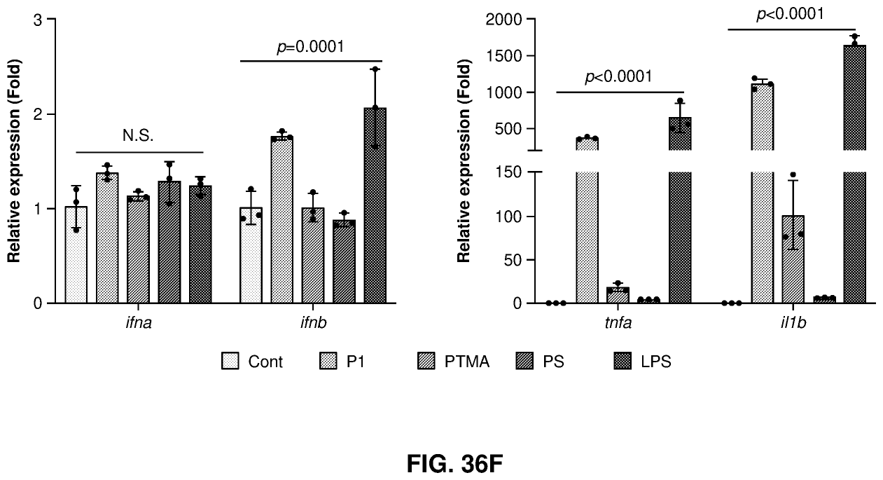

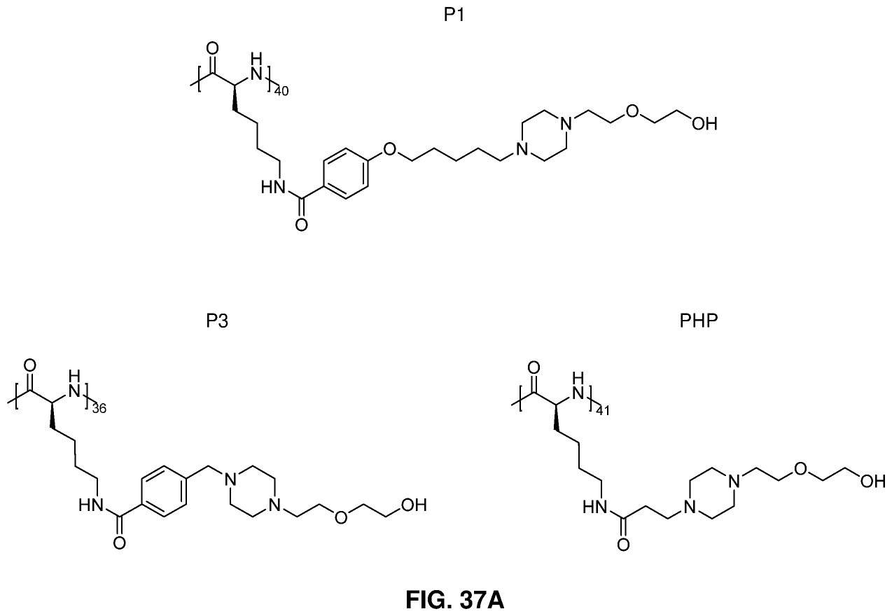

As can be seen in FIGS. 36B-36F, P1 induced ER stress (FIG. 36B) and mtDNA release (FIG. 36C) in bone marrow-derived macrophages (BMDMs) greater than PTMA (strongly cationic) and PS (anionic). The results showed that P1 also increased phagocytosis of EO771 breast cancer cells (FIG. 36D) and cross-presentation of the model antigen SIINFEKL-H2Kb (FIG. 36E) greater than PTMA and PS. The varying electrostatic charge of the analogues also affected gene expression of pro-inflammatory cytokines (FIG. 36F). The results of step 2 suggest a polypeptide comprising a moderate electrostatic charge induces greater pro-inflammatory responses in innate imunne cells.

Step 3 of the optimization process was modulation of the side chain of the polypeptide. Various analogues (P1, P3, and PUP) were prepared as shown in FIG. 34C, and their immunogenicity was determined via the mtDNA release assay and phagocytosis assay as described in Example 1.

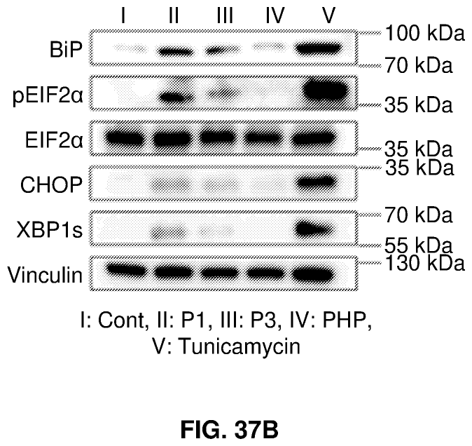

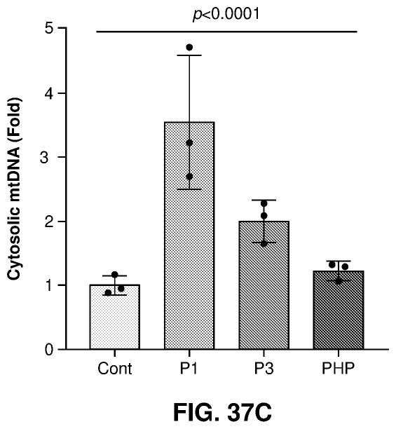

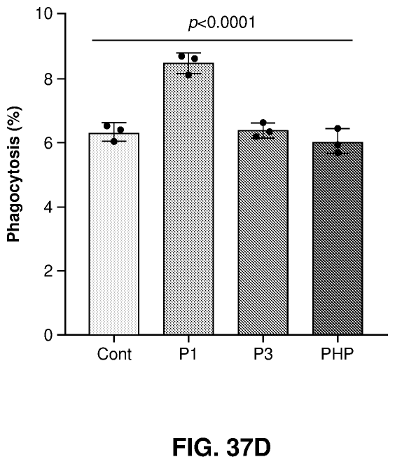

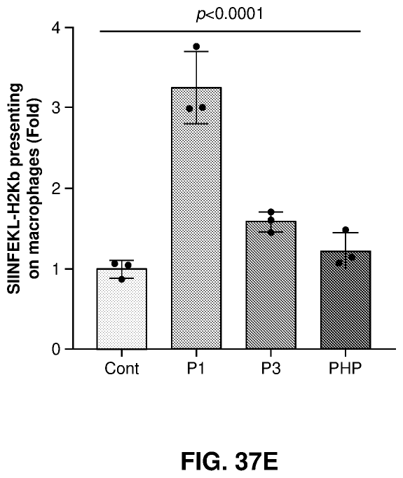

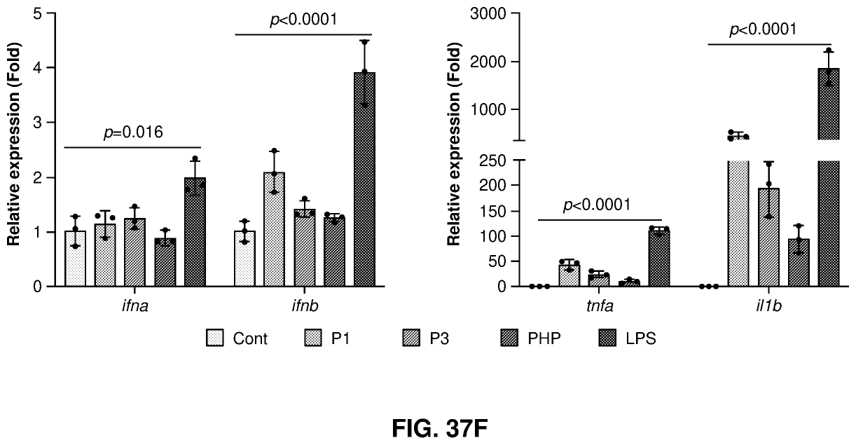

As can be seen in FIGS. 37B-37F, P1 induced ER stress (FIG. 37B) and mtDNA release (FIG. 37C) in bone marrow-derived macrophages (BMDMs) greater than P3 (mid-length side chain) and PS (short-length side chain). The results showed that P1 increased phagocytosis of EO771 breast cancer cells (FIG. 37D) and cross-presentation of the model antigen SIINFEKL-H2Kb (FIG. 37E) greater than P3 and PHP. The varying side chain length of the analogues also affected gene expression of pro-inflammatory cytokines (FIG. 37F). The results of step 3 suggest a polypeptide comprising a longer side chain tail length induces greater pro-inflammatory responses in innate immune cells.

### Example 9: Physiochemical Characterization and Antitumor Evaluation of Polypeptides In Vivo

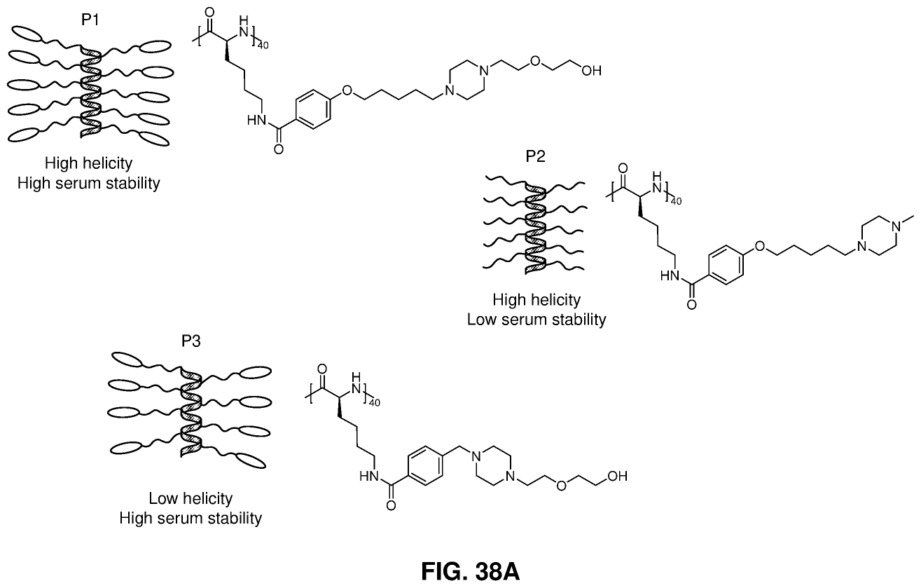

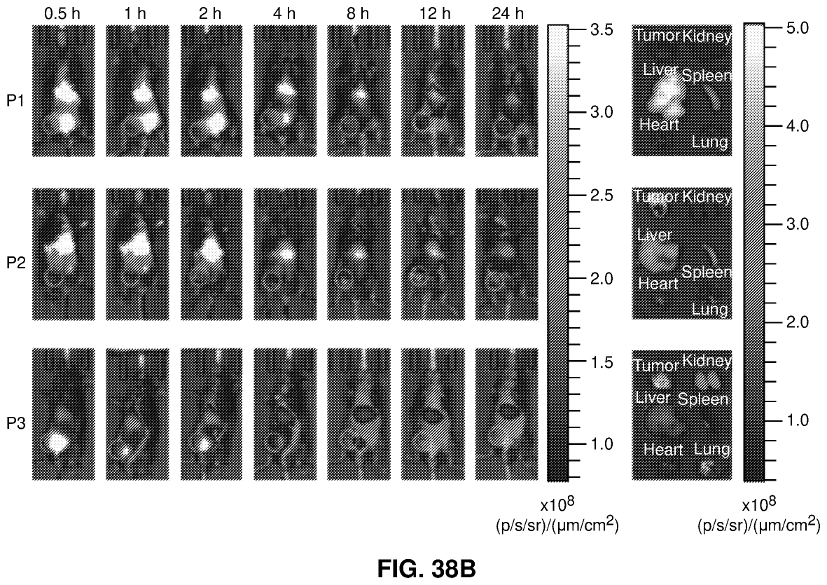

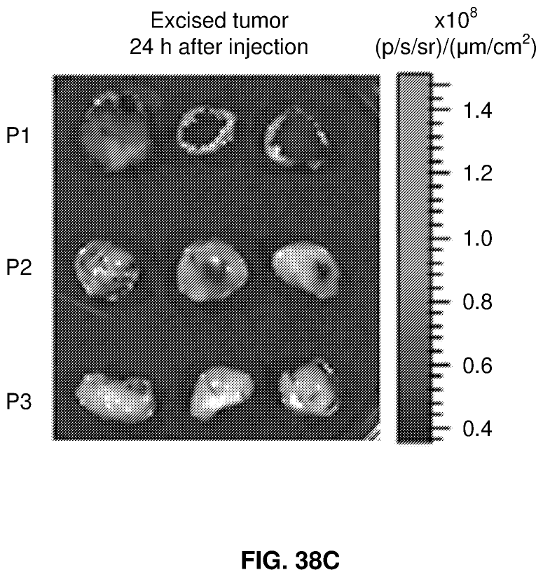

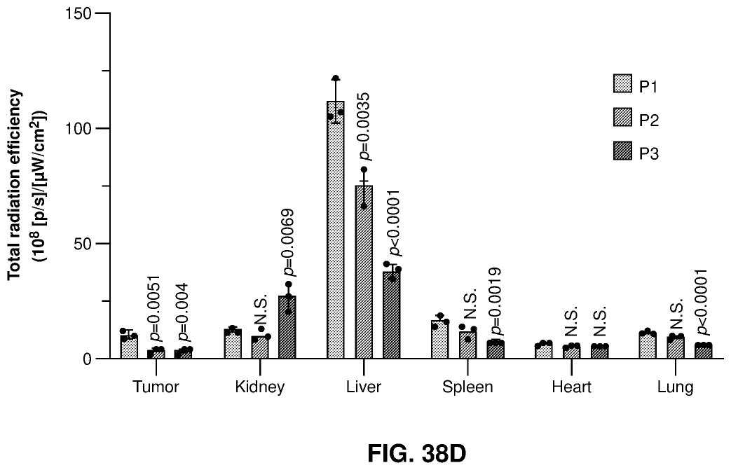

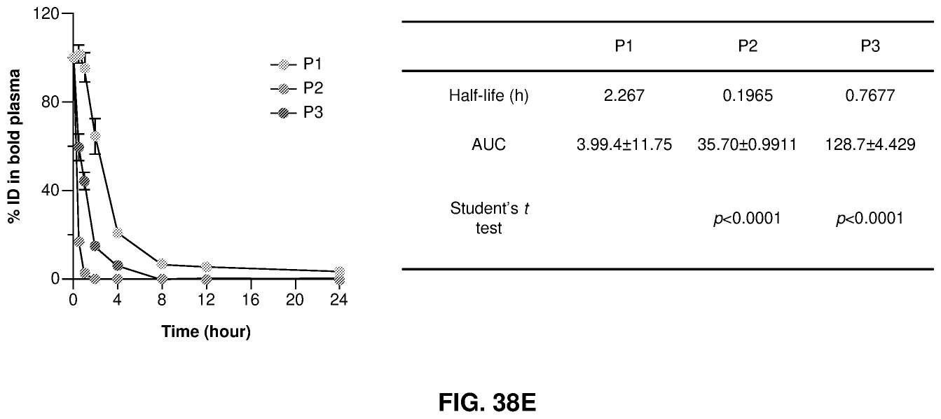

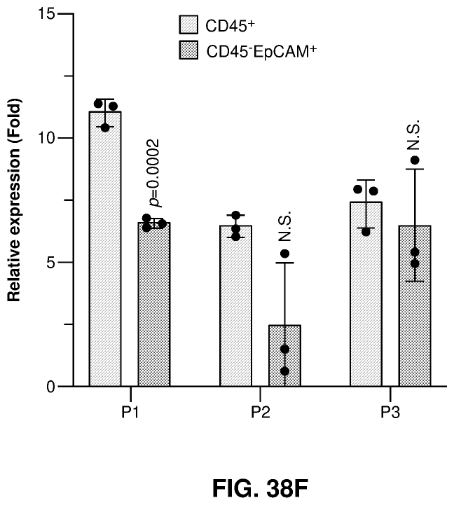

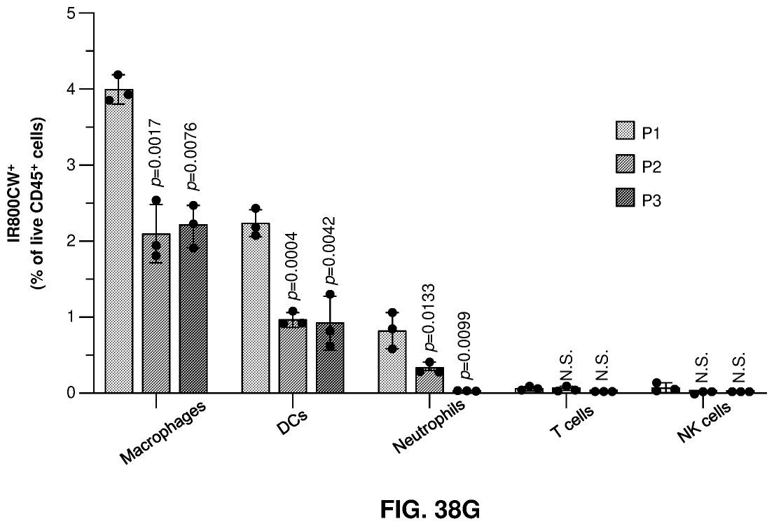

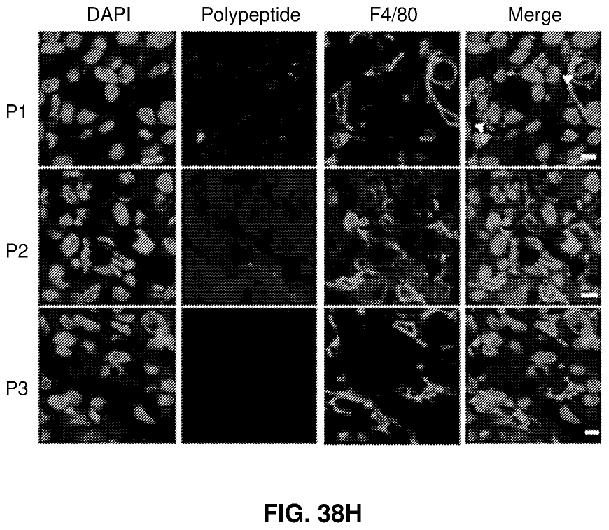

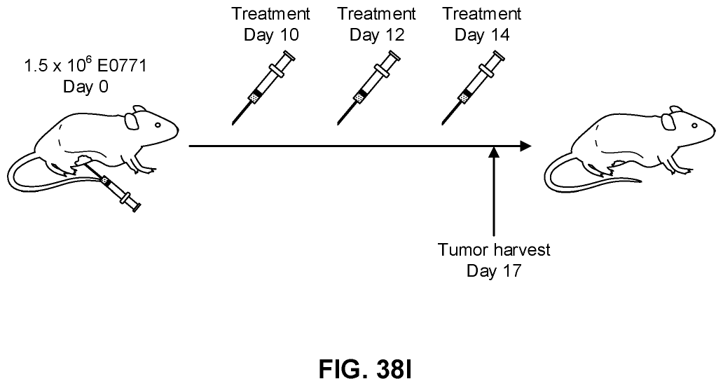

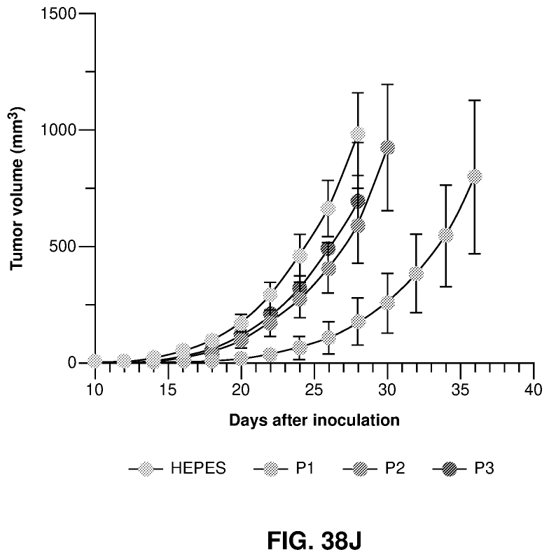

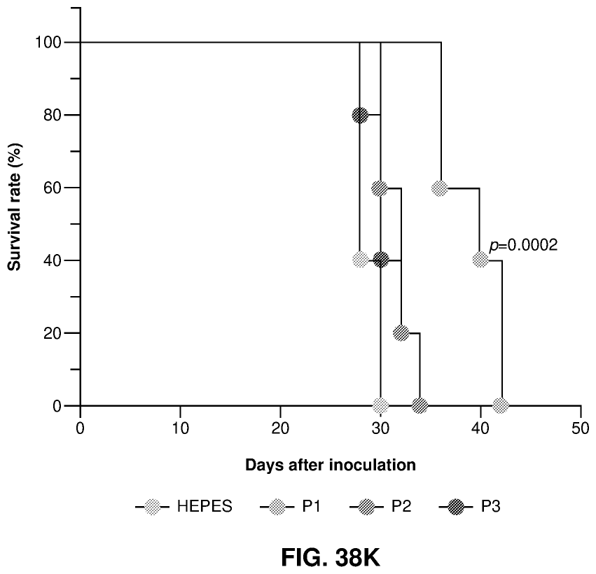

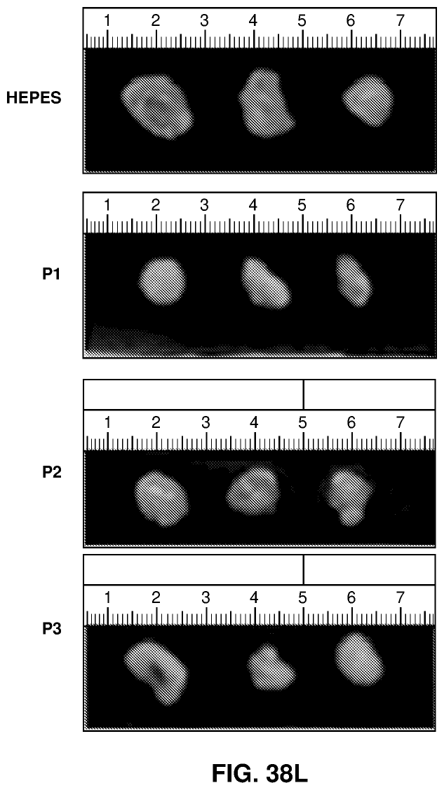

Three polypeptides, P1, P2, and P3, as shown in FIG. 38A, were tagged with IR800CW and injected intravenously (10 mg/kg) in EO771 tumor-bearing mice according to the treatment timeline shown in FIG. 38I.

FIG. 38B shows fluorescence images taken at 0.5 h, 1 h, 2 h, 4 h, 8 h, 12 h, and 24 h after intravenous injection of IR800CW-tagged P1, P2, or P3, and ex vivo fluorescence images of tumour and major organs harvested at 24 h after administration. FIG. 38C shows fluorescence images of EO771 tumour tissues excised at 24 h after treatment with IR800CW-tagged P1, P2 or P3. FIG. 38D shows quantification of total radiation efficiency (near infrared fluorescence signal) of tumour and major organs at 24 h after treatment. The time course of polypeptide concentrations in blood plasma after intravenous administration of IR800CW-tagged P1, P2, or P3 is shown in FIG. 38E. The half-life and area under the curve (AUC) values were calculated by GraphPrism.

FIGS. 38F and 38G show the measurement of IR800CW fluorescence signals in CD45⁺ cells (leukocytes), CD45⁻ EpCAM⁺ (EO771 tumour cells) (FIG. 38F), and immune cell subtypes (macrophages: CD11b⁺CD11c⁻F4/80⁺, dendritic cells [DCs]: CD11c⁺MHC-II⁺F4/80⁻, neutrophils: CD11b⁺CD11c⁻ MHC-II⁻Gr1⁺, T cells: CD3⁺, NK cells: CD3⁻NKp46⁺NK1.1⁺) (FIG. 38G) by flow cytometry of tumours 24 after treatment with IR800CW-tagged P1, P2, or P3. Immunofluorescence images (FIG. 38H) show that IR800CW-tagged P1 accumulates in macrophages and DCs within tumour microenvironments to a greater extent than IR800CW-tagged P2 or P3 (scale bar, 15 μm). The results show that P1 suppressed tumour growth and extended survival of tumour-bearing mice compared with P2 or P3 (FIG. 38J).

### Example 10: P1 Inhibits Tumor Growth and Extends Survival in Tumour-Bearing Mice

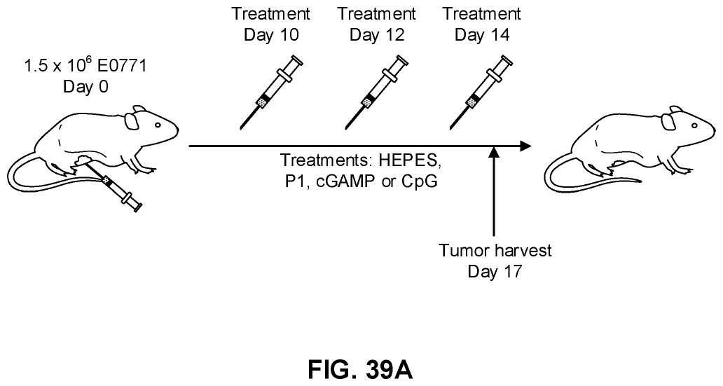

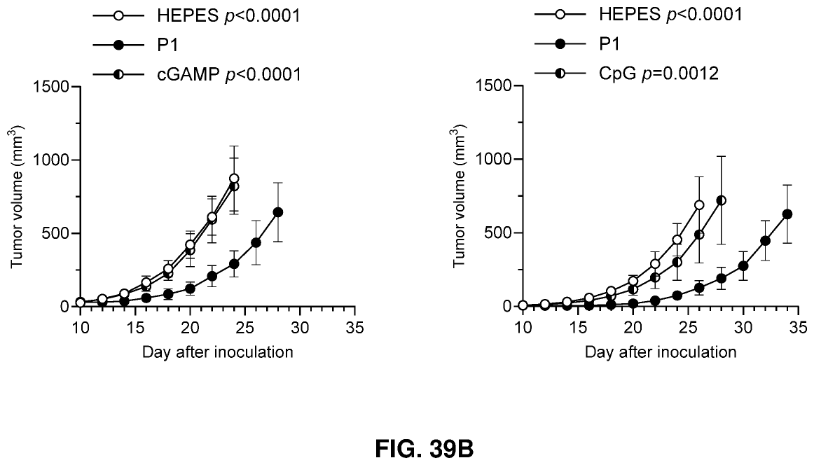

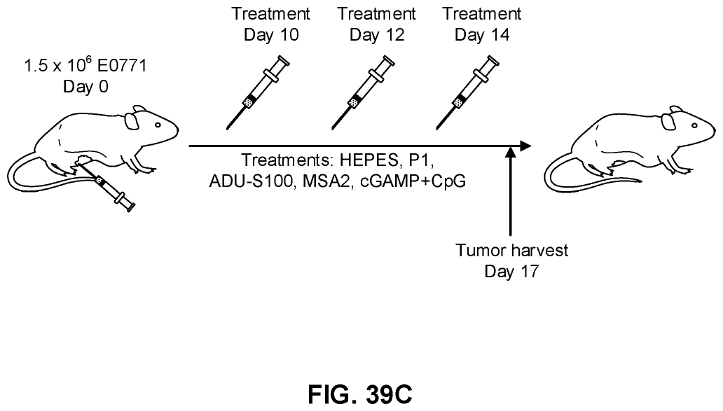

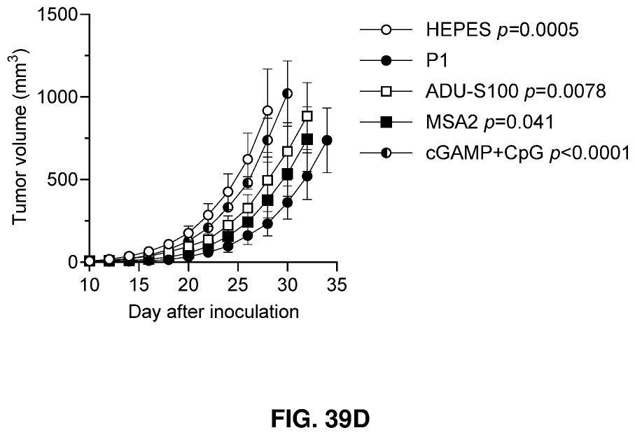

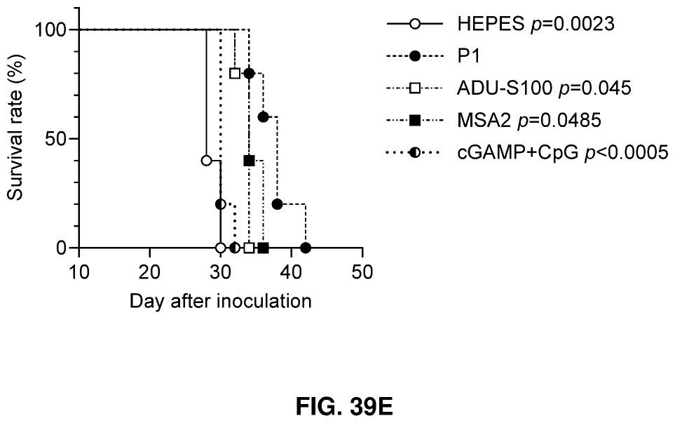

To evaluate the inhibition of tumor growth in mice, treatments of 10 mg/kg of HEPES, P1, cGAMP, and CpG were injected intravenously in EO771 tumor bearing mice according to the timeline shown in FIG. 39A. As can be seen in FIG. 39B, P1 treatment suppressed tumour growth of EO771 tumour-bearing mice to a greater extent than cGAMP, and CpG.

In a comparison study of tumor growth inhibition of P1 and STING agonists, treatments of 10 mg/kg of HEPES, P1, ADU-S100 (a synthetic STING agonist with thiol esters), MSA-2 (non-nucleotide STING agonist), or cGAMP+CpG (10 mg/kg cGAMP+10 mg/kg CpG) were injected intravenously in EO771 tumor bearing mice according to the timeline shown in FIG. 39C. The results show that P1 treatment suppressed tumour growth of EO771 tumour-bearing mice to a comparable extent as MSA-2 and to a greater extent than ADU-S100, and cGAMP+CpG (FIG. 39D).

### Example 11: P1 does not Induce Organ Toxicity or Generate Systemic Inflammation

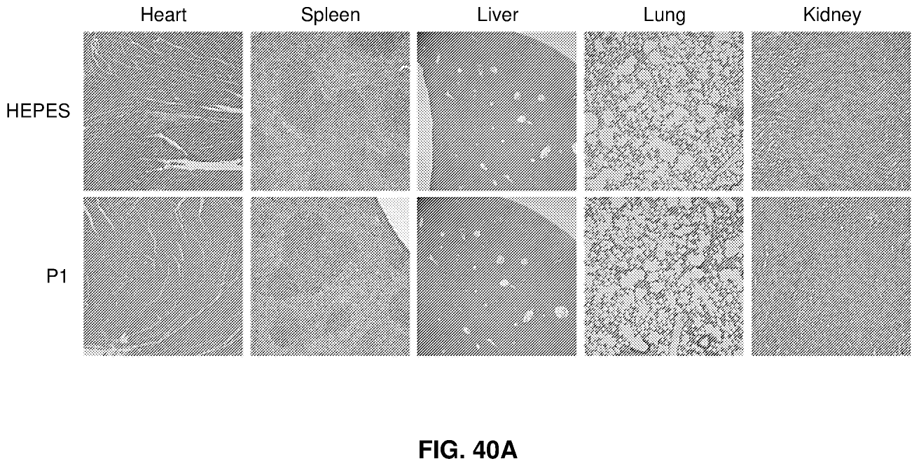

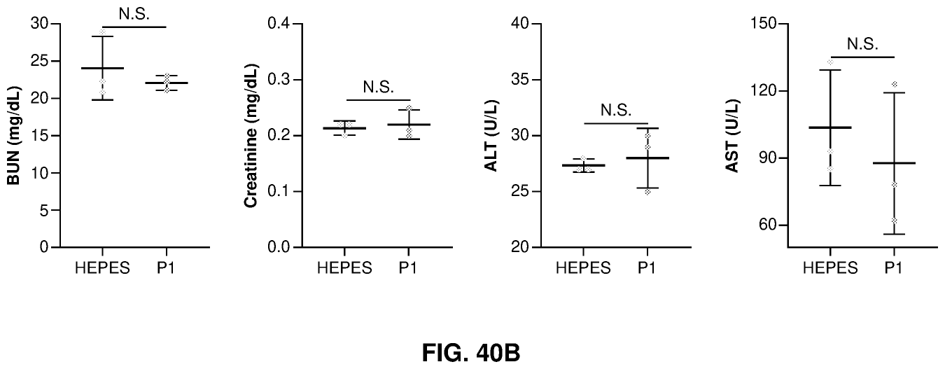

To evaluate induced organ toxicity, C57BL/6J mice were intravenously given HEPES or P1 (10 mg/Kg) three times every other day. Organs and blood were obtained 24 h after the last treatment. FIG. 40A shows histological images of heart, spleen, liver, lung, and kidney tissue, showing that P1 did not induce toxicity in the organs. FIG. 40B shows levels of blood urea nitrogen (BUN), creatinine, alanine aminotransferase (ALT), and aspartate aminotransferase (AST) in blood plasma of treated mice. The results show that P1 does not induce organ toxicity or alter the levels of BUN, ALT, AST in a significant way.

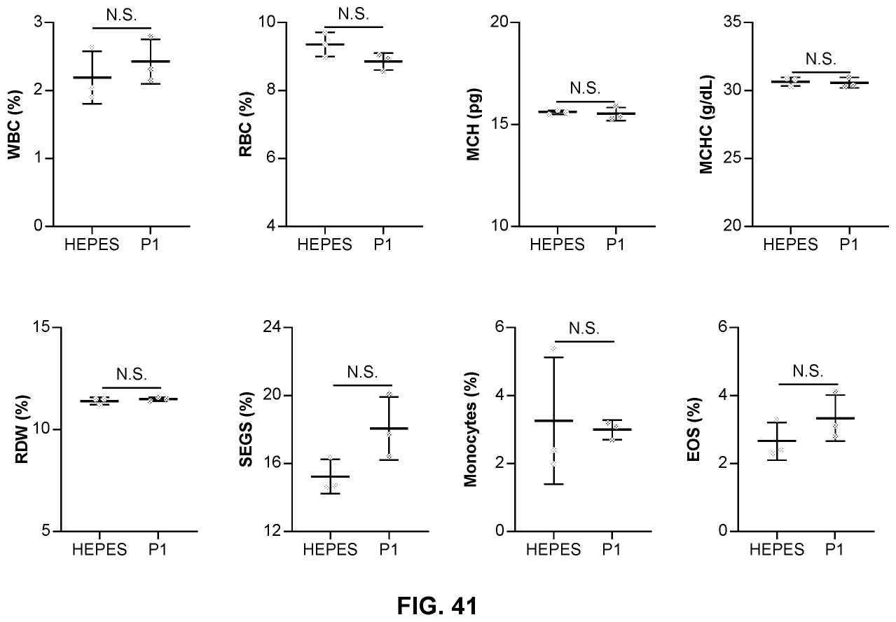

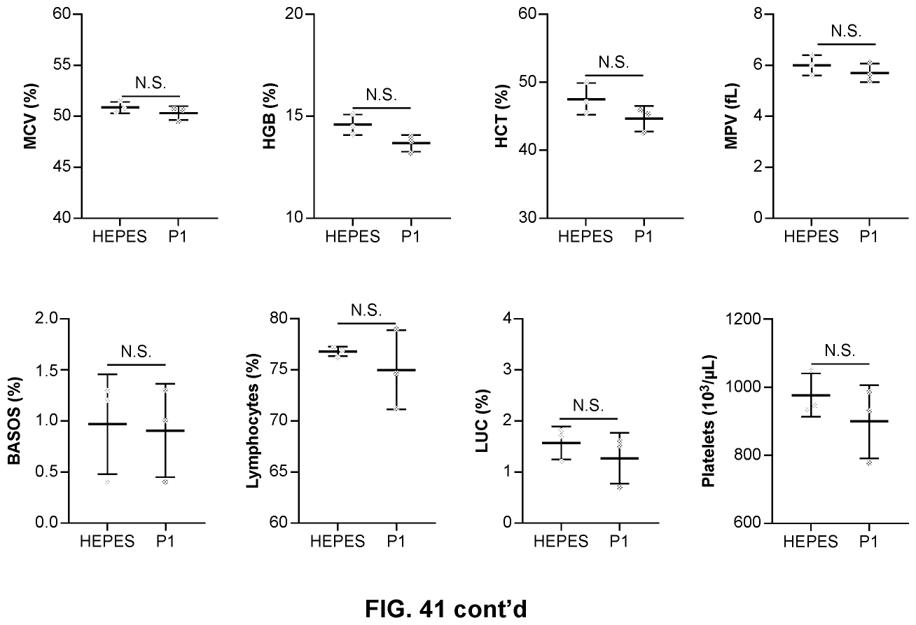

To evaluate systemic inflammation, a complete blood count was performed 24 h after the last treatment measuring the affect of P1 in comparison to HEPES on white blood cell (WBC), red blood cell (RBC), mean corpuscular hemoglobin (MCH), mean corpuscular volume (MCV), mean corpuscular hemoglobin concentration (MCHC), hemoglobin (HGB), hematocrit (HCT), mean platelet volume (MPV), red cell distribution width (RDW), segmental neutrophils (SEGS), eosinophils (EOS), basophils (BASOS), and large unstained cells (LUC) concentrations. FIG. 41 shows no significant difference in complete blood count was observed in mice 24 h after the last treatment, suggesting that P1 does not generate systemic inflammation.

### Example 12: P1 with αPD1 Combination Therapy

P1 treatments were given on Day 10, 12, and 14 after tumor inoculation while αPD1 treatments were given on Day 11, 13, and 15 after tumor inoculation. Tumors treated with P1+αPD1 were harvested on Day 17 to perform flow cytometry of tumor-infiltrating lymphocytes, immune cells within tumor draining lymph nodes, and splenocytes by staining the cells with corresponding antibodies or SIINFEKL-H2Kb tetramer.

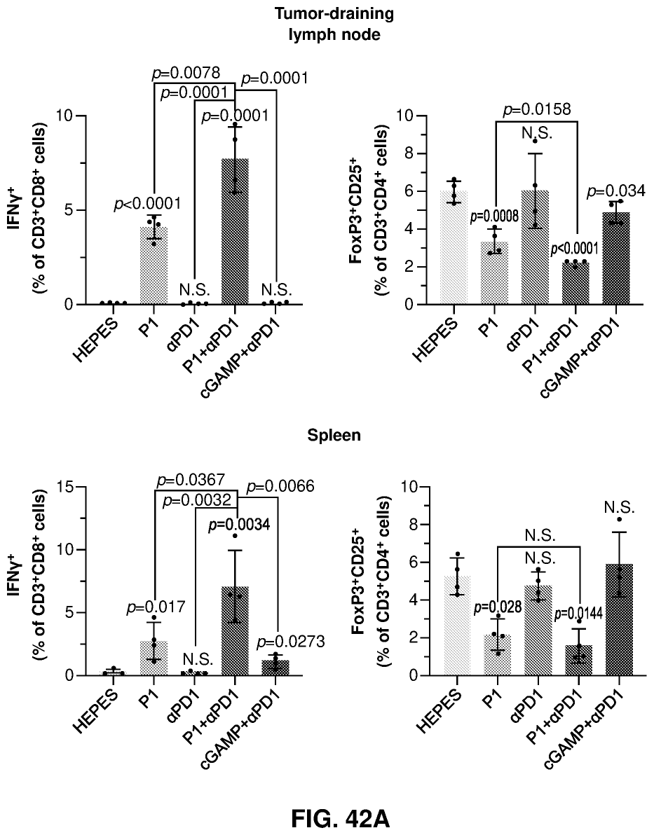

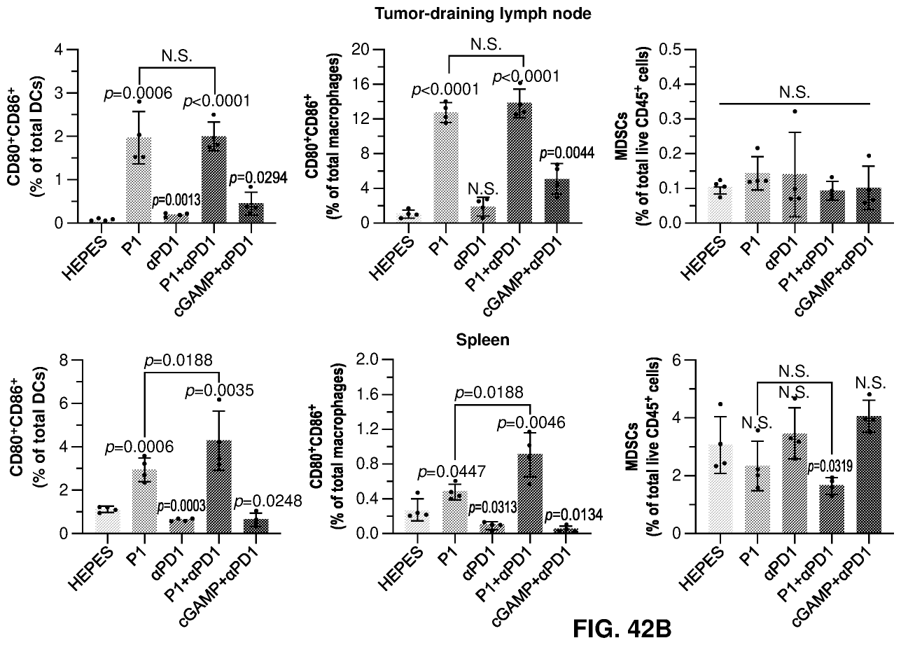

FIG. 42A shows that P1+αPD1 treatment increased the production of IFNγ from CD8⁺ T cells and decreased the population of Tregs in tumour-draining lymph nodes and spleen as compared with P1 and αPD1 as monotherapy, or cGAMP+αPD1. In addition, the combination treatment of P1+αPD1 increased the population of mature DCs and pro-inflammatory macrophages polarization, but maintained the numbers of MDSCs in tumour-draining lymph nodes and spleen, relative to P1, αPD1, or cGAMP+αPD1 treatments (FIG. 42B).

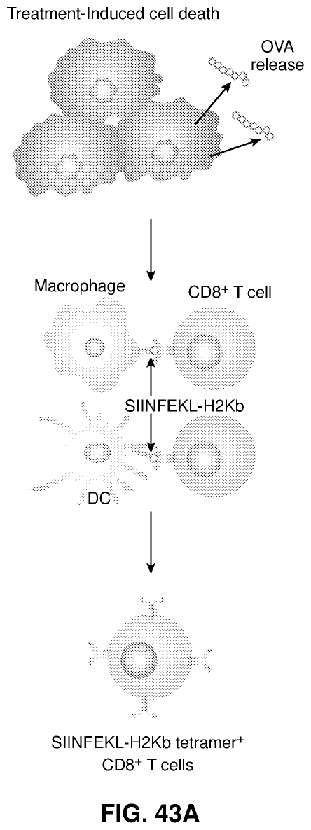

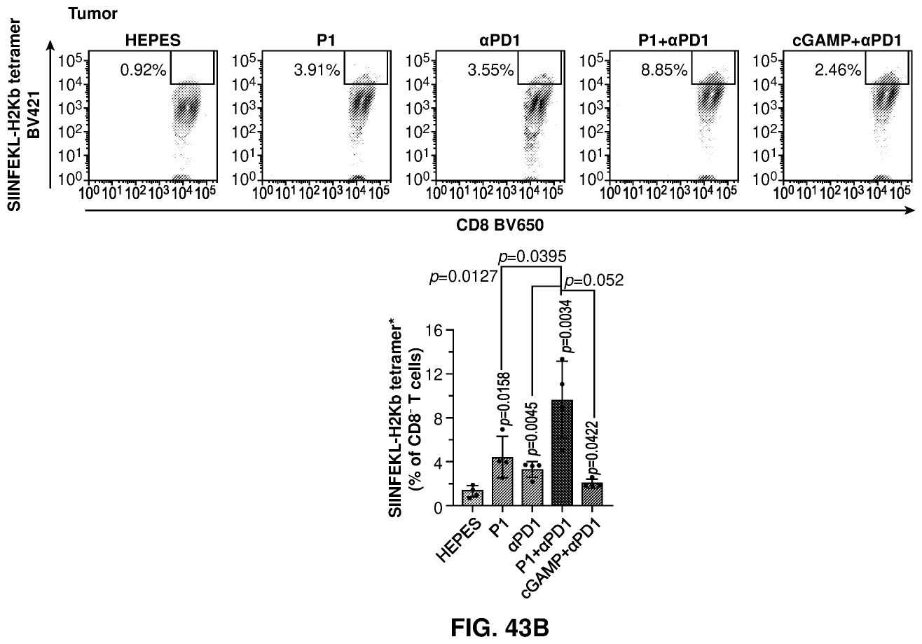

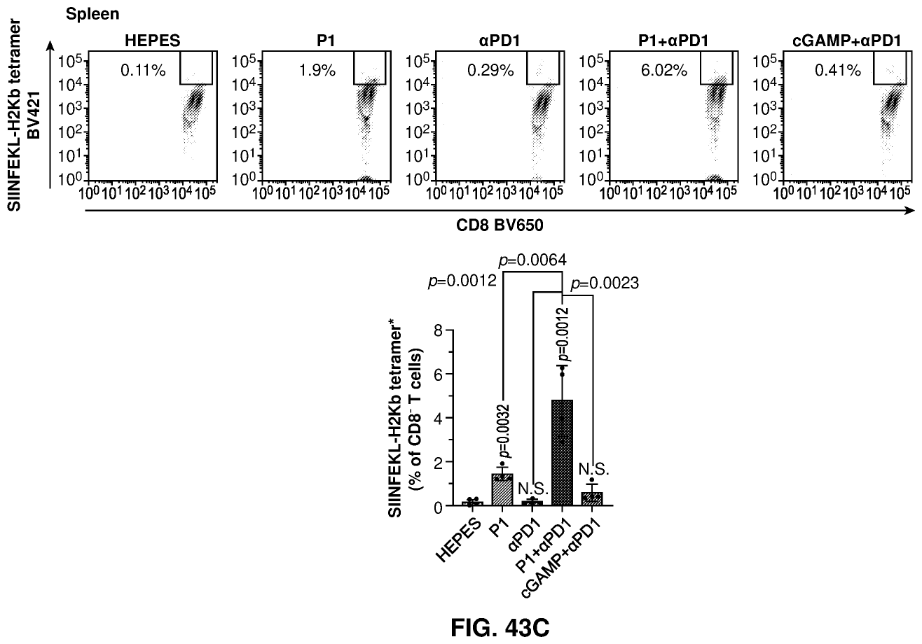

The combination treatment of P1+αPD1 also elicited tumor-specific T cell responses in solid tumors. P1+αPD1 increased SIINFEKL-H2Kb tetramer+CD8⁺ T cells within the tumour microenvironment and in spleen (FIG. 43B and FIG. 43C).

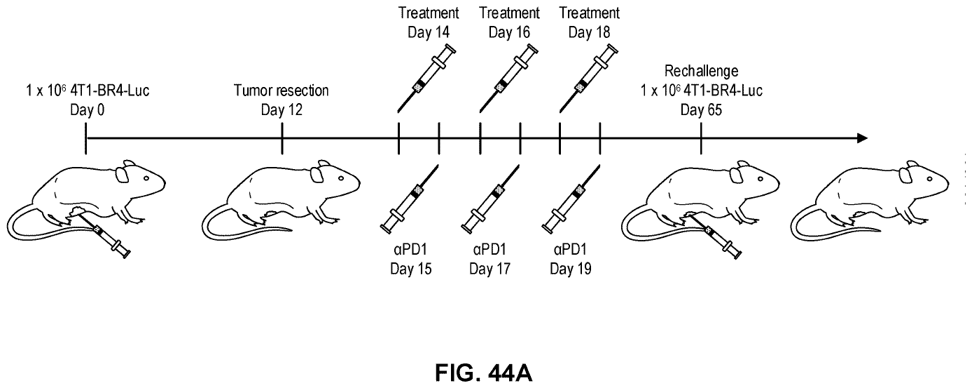

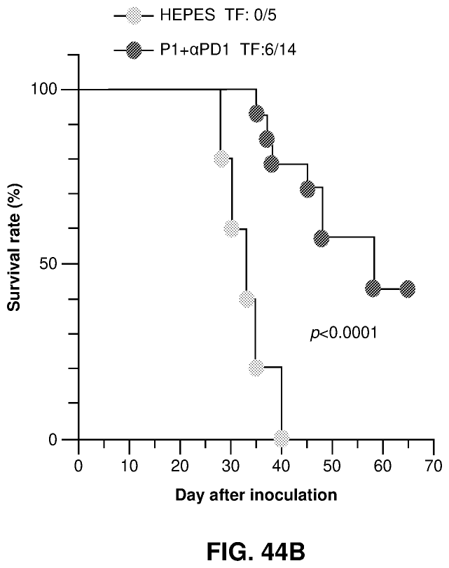

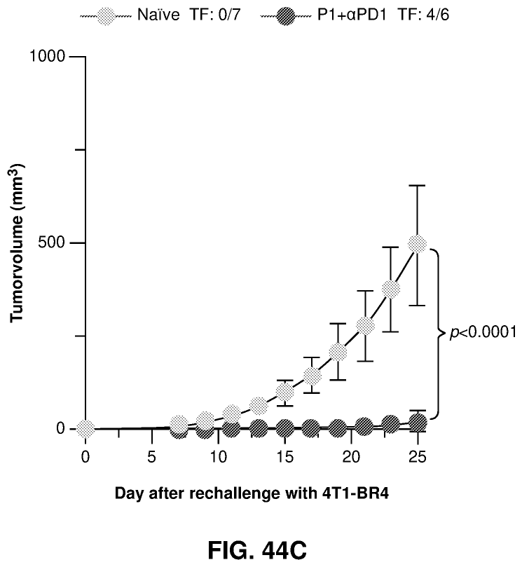

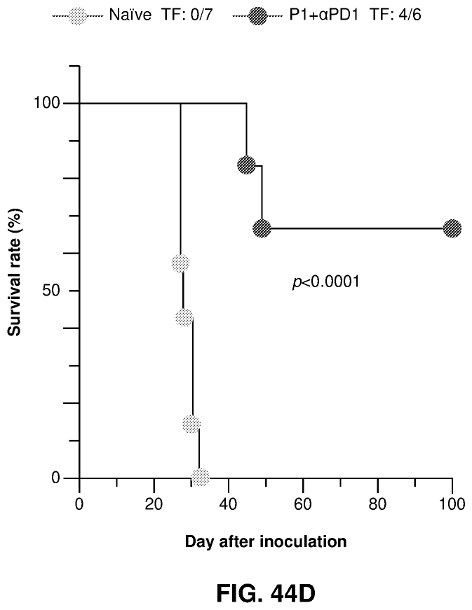

4T1-BR4 breast tumour bearing mice were treated with the P1+αPD1 combination therapy for tumour re-challenge according to the treatment schedule shown in FIG. 44A. 4T1-BR4 metastatic primary tumors were resected on day 12 after tumor inoculation. P1 treatments were given on Day 14, 16, and 18 after tumor inoculation while αPD1 treatments were given on Day 15, 17, and 19 after tumor inoculation. Animal survival of tumor-bearing mice with tumor metastases were observed until Day 65. The mice with long-term survival were rechallenged with 4T1-BR4 to confirm the generation of tumor-specific memory conferred by P1+αPD1 treatment. The further animal survival of mice rechallenged with 4T1-BR4 were observed until day 100 after rechallenge. The results show that treating immunized mice with P1+αPD1 led to suppressed re-occurrence of tumour establishment, thus increasing the mouse survival time (FIG. 44B, FIG. 44C, and FIG. 44D).

Having now fully described this invention, it will be understood by those of ordinary skill in the art that the same can be performed within a wide and equivalent range of conditions, formulations and other parameters without affecting the scope of the invention or any aspect thereof.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

All patents, patent applications, and other publications cited herein are fully incorporated by reference herein in their entirety.

What is claimed is:

1. A cationic polypeptide-based adjuvant comprising a poly-lysine peptide conjugated to at least one amine containing building block.

2. A polypeptide-based adjuvant comprising the structure of Formula 1: 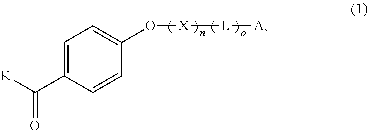
wherein:
a. K is a poly-lysine peptide;
b. A is an amine containing building block, an immunogenic agent, an imaging agent, a therapeutic agent, a stabilizing agent, targeting agent, CH₃ or is absent;
c. X is CH₂ or —CH₂CH₂O;
d. L is a linker;
wherein n is an integer from 1 to 10, and
is an integer from 0 to 1.

3. The polypeptide-based adjuvant according to claim 2, wherein L is selected from the group consisting of substituted or unsubstituted C₁-C₁₀ alkyl, —CH₂CH₂O, streptavidin-biotin, carboxylic acid-amine, carbonates, disulfide bonds, azide-alkyne, azide-DBCO, thiolenes, maleimides, enzyme cleavable peptides, and combinations thereof.

4. The polypeptide-based adjuvant according to any one of claims 1-3, wherein the amine containing building block is attached to the adjuvant by the amine and comprises a compound selected from the group consisting of substituted or unsubstituted heterocyclic compounds, substituted or unsubstituted heteroaryl compounds, linear or branched alkyl amines, substituted or unsubstituted aza-crown ethers, and combinations thereof.

5. The polypeptide-based adjuvant according to any one of claims 1-4, wherein the amine containing building block comprises a compound selected from the group consisting of piperazine, 1-methylpiperazine, 1-(2-hydroxyethyl)piperazine, 1-[2-(2-hydroxyethoxy)ethyl]piperazine, morpholine, piperidine, 1-aza-12-crown-4, 1-aza-15-crown-5,1-aza-18-crown-6, dimethyl amine, diethyl amine, dipropyl amine, dibutyl amine, trimethyl amine, triethyl amine, tripropyl amine, tributyl amine, 1-benziimidazole, and combinations thereof.

6. The polypeptide-based adjuvant according to any one of claims 1-4, wherein the amine containing building block comprises one or more C₁-C₆ alkyl, halogen, cyano, or hydroxy groups.

7. The polypeptide-based adjuvant according to any one of claims 1-6 wherein the poly-lysine peptide comprises a monomer comprising a substituted oxybenzoyl-L-lysine.

8. The polypeptide-based adjuvant according to any one of claims 1-7, wherein the poly-lysine peptide comprises a compound selected from the group consisting of N⁶-4-(2-chloroethyl)oxybenzoyl-L-lysine, N⁶-4-(3-chloropropyl)oxybenzoyl-L-lysine, N⁶-4-(4-chlorobutyl)oxybenzoyl-L-lysine, N⁶-4-(5-chloropentyl)oxybenzoyl-L-lysine, N⁶-4-(6-chlorohexyl)oxybenzoyl-L-lysine, N⁶-4-(7-chloroheptyl)oxybenzoyl-L-lysine, and N⁶-4-(8-chlorooctyl)oxybenzoyl-L-lysine.

9. The polypeptide-based adjuvant according to any one of claims 1-8, wherein the poly-lysine peptide has a molecular weight (g/mol) ranging from about 1,000 to about 1,000,000.

10. The polypeptide-based adjuvant according to any one of claims 1-9, wherein the poly-lysine peptide comprises one or more compounds selected from the group consisting of immunogenic agents, therapeutic agents, imaging agents, stabilizing agents, targeting agents, linkers, and combinations thereof.

11. The polypeptide-based adjuvant according to claim 10, wherein the poly-lysine peptide is conjugated to a therapeutic agent by a cleavable linker, or encapsulates a therapeutic agent.

12. The polypeptide-based adjuvant according to claim 11, wherein the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, mRNA, siRNA, shRNA, plasmid DNA, CpG oligonucleotides, and combinations thereof.

13. The polypeptide-based adjuvant according to claim 1 or 2, wherein the polypeptide-based adjuvant is conjugated to an imaging agent by a cleavable linker or encapsulates an imaging agent.

14. The polypeptide-based adjuvant according to claim 13, wherein the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof.

15. The polypeptide-based adjuvant according to any one of claims 1-14, wherein the polypeptide-based adjuvant is conjugated to a stabilizer.

16. The polypeptide-based adjuvant according to claim 15, wherein the stabilizer is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof.

17. The polypeptide-based adjuvant according to any one of claims 1-16, wherein the polypeptide-based adjuvant is conjugated to a targeting molecule.

18. The polypeptide-based adjuvant according to claim 17, wherein the targeting molecule is selected from the group consisting of antibodies, nanobodies, aptamers, small molecule-based targeting ligands, and combinations thereof.

19. The polypeptide-based adjuvant according to claim 17 or 18, wherein the targeting agent is attached to the polypeptide-based adjuvant by a linker molecule.

20. The polypeptide-based adjuvant according to claim 19, wherein the linker molecule is selected from the group consisting of streptavidin-biotin, carboxylic acid-amine, carbonates, disulfide bonds, azide-alkyne, azide-DBCO, thiolenes, maleimides, enzyme cleavable peptides, and combinations thereof.

21. The polypeptide-based adjuvant according to any one of claims 1-20 wherein the poly-lysine peptide comprises:
a. N⁶-4-(2-hydroxyethyl)oxybenzoyl-L-lysine;
b. N⁶-4-(3-hydroxypropyl)oxybenzoyl-L-lysine;
c. N⁶-4-(4-hydroxybutyl)oxybenzoyl-L-lysine;
d. N⁶-4-(5-hydroxypentyl)oxybenzoyl-L-lysine;
e. N⁶-4-(6-hydroxyhexyl)oxybenzoyl-L-lysine;
f. N⁶-4-(7-hydroxyheptyl)oxybenzoyl-L-lysine; or
g. N⁶-4-(8-hydroxyoctyl)oxybenzoyl-L-lysine.

22. The polypeptide-based adjuvant according to any one of claims 1-21, wherein the polypeptide based adjuvant is capable of inducing an anti-tumor immune response.

23. The polypeptide-based adjuvant according to any one of claims 1-22, wherein the polypeptide is helical.

24. The polypeptide-based adjuvant according to any one of claims 1-23, wherein the polypeptide-based adjuvant is:

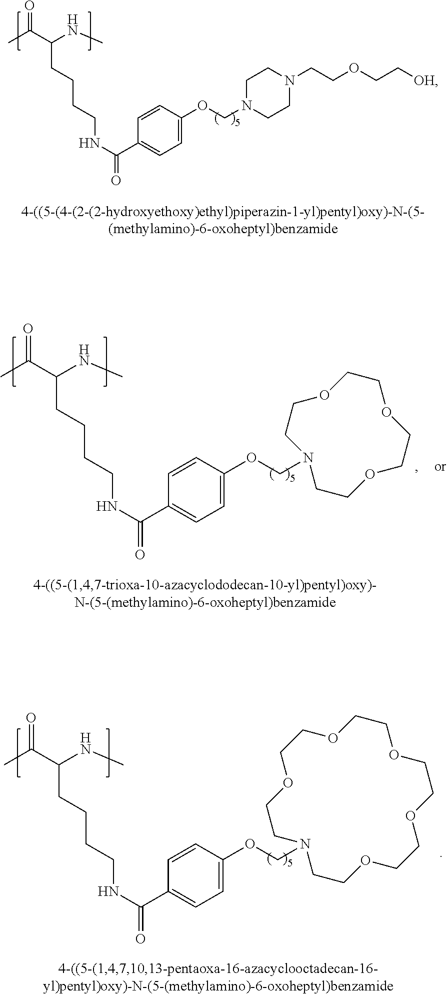

25. A composition comprising the polypeptide-based adjuvant according to any one of claims 1-24.

26. The composition according to claim 25, further comprising an excipient or carrier.

27. The composition according to claim 26, wherein the excipient or carrier comprises an anionic polymer.

28. The composition according to any one of claims 25-27, wherein the composition is a nanoparticle comprising the polypeptide-based adjuvant.

29. A pharmaceutical composition comprising the polypeptide-based adjuvant according to any one of claims 1-28 and a pharmaceutically acceptable excipient or a pharmaceutically acceptable carrier.

30. The pharmaceutical composition according to claim 29, wherein the pharmaceutically acceptable excipient or carrier comprises an anionic polymer.

31. The composition according to any one of claims 25-28 or the pharmaceutical composition according to any one of claims 29-30, wherein the poly-lysine peptide comprises one or more compounds selected from the group consisting of immunogenic agents, therapeutic agents, imaging agents, stabilizing agents, targeting agents, linkers, and combinations thereof.

32. The composition according to any one of claims 26-28 or the pharmaceutical composition according to any one of claims 29-30, wherein the carrier is succinylated dextran.

33. The composition or pharmaceutical composition according to claim 31, wherein the immunogenic agent is selected from the group consisting of substituted or unsubstituted piperazine, morpholine, piperadine, aza-crown ethers, linear or branched alkyl amines, substituted or unsubstituted benzimidazole, and combinations thereof.

34. The composition or pharmaceutical composition according to claim 31, wherein the therapeutic agent is selected from the group consisting of doxorubicin, paclitaxel, cisplatin, carboplatin, camptothecin, chorine e6, methotrexate, temzolomide, irinotecan, doxetacel, 5-FU, miRNA-155-5p, miRNA-p53, miRNA-PTEN, VEGF siRNA, STAT3 siRNA, 2′,3′-cyclic GMP-AMP, 2′,-3′-cyclic diGMP, diABZI STING agonist, CpG oligomer, R848, motilomod, GS9620, imatinib, UNC2025, PLX3397, BLZ945, IACS-8803 STING agonist, ML-RR-S2-CDA STING agonist, MSA STING agonist, Poly:C, calcium oxide nanoparticles, lipopolysaccharide, mRNA, siRNA, shRNA, plasmid DNA, CpG oligonucleotides, and combinations thereof.

35. The composition or pharmaceutical composition according to claim 31, wherein the imaging agent is selected from the group consisting of iron oxide, gadolinium, iodide, FDG, radio-isotopes, Cy5.5, Cy7, IR8000CW, gold nanoparticles, carbon nanotubes, graphene oxide, semi-conducting polymers, manganese oxide nanoparticles, ruthenium(II)-sonosensitizers, and combinations thereof.

36. The composition or pharmaceutical composition according to claim 31, wherein the stabilizing agent is selected from the group consisting of polyethylene glycol, poly(2-oxazoline), glycol chitosan, chitosan, hyaluronic acid, beta-glucan, beta-cyclodextrin, dextran, mannan, poly-L-glutamate, polyacrylic acid, and combinations thereof.

37. The composition or pharmaceutical composition according to claim 31, wherein the targeting agent is selected from the group consisting of antibodies, nanobodies, aptamers, small molecules, and combinations thereof.

38. The composition or pharmaceutical composition according to claim 37, wherein the targeting agent is conjugated to the poly-lysine peptide by a linker.

39. The composition or pharmaceutical composition according to claim 38, wherein the linker is selected from the group consisting of substituted or unsubstituted C₁-C₁₀ alkyl, —CH₂CH₂O, streptavidin-biotin, carboxylic acid-amine, carbonates, disulfide bonds, azide-alkyne, azide-DBCO, thiolenes, maleimides, enzyme cleavable peptides, and combinations thereof.

40. A vaccine comprising a polypeptide-based adjuvant according to any one of claims 1-24, and a pharmaceutically acceptable carrier or diluent suitable for use in a vaccine.

41. The vaccine according to claim 40, wherein the vaccine further comprises an immunogenic compound.

42. The vaccine according to claim 41, wherein the immunogenic compound is selected from the group consisting of piperazine, morpholine, piperadine, aza-crown ether, alkyl amine, benzimidazole, and combinations thereof.

43. A kit comprising: (i) a polypeptide-based adjuvant according to any one of claims 1-24; (ii) a composition according to any one of claims 25-28 or 31-39; (iii) a pharmaceutical composition according to any one of claims 29-39; or (iv) a vaccine according to any one of claims 40-42.

44. A method of treating cancer in a subject in need thereof, comprising administering to the subject an effective amount of the composition according to any one of claims 25-28 or 31-39, the pharmaceutical composition according to any one of claims 29-39, or a vaccine according to any one of claims 40-42.

45. The method of claim 44, wherein the cancer is selected from the group consisting of a sarcoma, melanoma, carcinoma, glioblastoma or other solid tumors including breast, prostate, lung, kidney and pancreatic tumors.

46. The method of claim 45, wherein the cancer is breast cancer or a breast cancer metastases.

47. The method of claim 44, further comprising administering a therapeutic agent selected from the group consisting of an anti-PD-1 inhibitor, an anti-PD-L1 inhibitor, an anti-CTLA4 inhibitor, an anti-CD47 inhibitor, an indole 2,3-dioxygenase inhibitor, and combinations thereof.

48. A method for inducing an immune response or enhancing an immune response in a subject in need thereof, comprising administering to the subject an effective amount of the composition according to any one of claims 25-28 or 31-39, the pharmaceutical composition according to any one of claims 29-39, or a vaccine according to any one of claims 40-42.

49. A method of immunizing or conferring a protective immunity against a cancer in a subject in need thereof, comprising administering to the subject the polypeptide-based adjuvant according to any one of claims 1-24, the composition according to any one of claims 25-28 or 31-39, the pharmaceutical composition according to any one of claims 29-39, or a vaccine according to any one of claims 40-42.

50. A method of delivering a small molecule compound or a polynucleotide to a subject in need thereof, comprising administering the polypeptide-based adjuvant according to any one of claims 1-24, the composition according to any one of claims 25-28 or 31-39, or the pharmaceutical composition according to any one of claims 29-39.

51. The method of claim 50, wherein the polypeptide-based adjuvant is conjugated to or encapsulates the small molecule compound or a polynucleotide.

52. The methods of any one of claims 44-51, wherein the polypeptide-based adjuvant according to any one of claims 1-24, the composition according to any one of claims 25-28 or 31-39, or the pharmaceutical composition according to any one of claims 29-39 is administered orally, intravenously, intraperitoneally, intratumorally, intramuscularly, subcutaneously, or intrathecally.

53. A method of treating a disease or disorder in a subject in need thereof, comprising administering to the subject an effective amount of a vaccine according to any one of claims 40-42.

54. A method of making the polypeptide-based adjuvant according to any one of claims 1-24, the composition according to any one of claims 25-28 or 31-39, or the pharmaceutical composition according to any one of claims 29-39, comprising:
(a) making a poly-lysine peptide by synthesizing a lysine monomer followed by polymerization to form a poly-lysine peptide,
(b) conjugating an amine containing building block, and optionally
(c) adding the polypeptide-based adjuvant to an anionic polymer in varying weight ratios to form a nanoparticle.
